(19) United States
(12) Patent Application Publication
(10) Pub. No.: US 2026/0216225 A1
(43) Pub. Date: Jul. 30, 2026

(54) INHIBITION OF ZBTB7A

(71) Applicant: Icahn School of Medicine at Mount Sinai, New York, NY (US)

(72) Inventors: Sasha Fulton, New York, NY (US); Ian S. Maze, New York, NY (US)

(21) Appl. No.: 19/141,869

(22) PCT Filed: Dec. 21, 2023
(86) PCT No.: PCT/US2023/085335

§ 371 Date: Jun. 20, 2025

Related U.S. Application Data
(60) Provisional application No. 63/434,403, filed on Dec. 21, 2022.

(51) Int. Cl. A61K 31/7105; A61K 31/711; A61K 35/76; A61K 38/46; A61K 48/00; A61P 25/00; C12N 7/00

(52) U.S. Cl. CPC A61K 31/7105; A61K 31/711; A61K 35/76; A61K 38/465; A61K 48/005; A61P 25/00; C12N 7/00; C12N 2310/11; C12N 2750/14121

(57) ABSTRACT

Provided herein are compositions and methods for treating various disorders by inhibition of zinc finger and BTB domain-containing protein 7A (ZBTB7A). The compositions and methods provided herein may be useful for treating psychiatric disorders, for example, Major Depressive Disorder (MDD), Bipolar Disorder, Dysthymia, Post-traumatic Stress Disorder (PTSD), Sub-stance-induced mood disorder, Generalized Anxiety Disorder, Social Phobia, Panic Disorder, or Mood Disorder Due to a General Medical.

### CROSS-REFERENCE TO RELATED APPLICATIONS

This application claims the benefit of the filing date of U.S. Provisional Application No. 63/434,403, filed Dec. 21, 2022, the contents of which are herein incorporated by reference in their entirety.

### FEDERALLY SPONSORED RESEARCH OR DEVELOPMENT

This invention was made with government support under grant numbers P50 MH096890 and R01 MH116900, awarded by The National Institutes of Health. The government has certain rights in the invention.

### TECHNICAL FIELD

The present disclosure relates to treating various disorders by inhibition of zinc finger and BTB domain-containing protein 7A (ZBTB7A).

### BACKGROUND

Emerging evidence indicates that glial neuroinflammation can be a contributor to the etiology of psychiatric disorders, including, for example, Major Depressive Disorder (MDD). MDD is a leading cause of disability worldwide and can involve corticolimbic network disruption associated with recurrent episodes of, for example, negative affect, cognitive impairment, somatic deficits, and anhedonia. Disease-related cellular phenotypes can be dictated by spatiotemporal gene expression programs induced by transcription factors (TFs) that interact with their corresponding cis-regulatory DNA elements in a cell-type specific manner.

The orbitofrontal cortex (OFC) is a frontal cortical structure that can integrate internal homeostatic states, emotion, and valenced stimuli to flexibly guide behavior. Human imaging studies have identified significant OFC dysfunction in MDD patients, with OFC hyperactivity correlating with severity of symptoms, suicidality, and pathogenic trajectories of the disorder. Despite its involvement in MDD, the molecular and cellular substrates underlying these functional alterations in OFC remain poorly understood.

Overall risk for psychiatric disorders, including MDD, can be influenced by complex interactions between genetic and environmental factors that influence epigenetic regulation of neuroplasticity and stress pathways. These mechanisms can be specific to the distinct regulatory context within regionally-defined brain cell-types. The chromatin remodeling protein ZBTB7A, which coordinates wide-ranging cellular activation programs, including NF-κB inflammatory transcription in cells of the OFC, can have a role in driving glial neuroinflammation. There is therefore a need for composition and methods for the inhibition of ZBTB7A for the treatment of psychiatric disorders relating to OFC dysfunction.

### SUMMARY

The present disclosure is based, in part, on the discovery that inhibition of ZBTB7A can reduce pathophysiological processes associated with neuroinflammation in the OFC and reduce behavioral deficits associated with chronic stress in the context of psychiatric disorders, including but not limited to Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition. In particular, astrocytic ZBTB7A may act as a pathogenic driver of chronic inflammation in psychiatric disorders by reversing the normal adaptive mechanisms that promote stress resilience. Inhibition of ZBTB7A can reverse chromatin remodeling, reactive astrocyte transcription, and behavioral deficits associated with chronic stress in the context of psychiatric disorders.

In one aspect, provided herein are methods of treating a psychiatric disorder, the method including identifying a subject as having the psychiatric disorder and administering to the subject an effective dose of a composition that reduces levels of ZBTB7A in cells of the subject. In some embodiments, the psychiatric disorder is one of Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition. In some embodiments, the composition includes an inhibitory agent that targets ZBTB7A in cells of the subject. In some embodiments, the composition includes an inhibitory agent that reduces expression of ZBTB7A in cells of the subject. In some embodiments, the inhibitory agent is an antisense oligonucleotide (ASO). In some embodiments, the inhibitory agent is a nucleic acid-guide nuclease. In some embodiments, the inhibitory agent is an RNAi agent. In some embodiments, the RNAi agent is an siRNA, an shRNA, or a miRNA. In some embodiments, the RNAi agent is a miRNA encoded by a DNA sequence comprising a sequence selected from the group consisting of SEQ ID NO: 3; SEQ ID NO: 4; SEQ ID NO: 5; SEQ ID NO: 6; SEQ ID NO: 7; SEQ ID NO: 8; SEQ ID NO: 9; or SEQ ID NO: 10. In some embodiments, the miRNA is expressed by a recombinant adeno-associated (rAAV) virus. In some embodiments, the inhibitory agent is administered with a pharmaceutically acceptable carrier and/or diluent. In some embodiments, the cells of the subject are in the orbitofrontal cortex (OFC) of the subject. In some embodiments, the cells of the subject are astrocyte cells. In some embodiments, levels of ZBTB7A are reduced by 10%, 20%, 30%, 40%, 50%, 60%, 70%, 80%, 90%, or 100% in the cells of the subject.

In another aspect, provided herein are methods of treating a psychiatric disorder, including administering to a subject having the psychiatric disorder an effective dose of a composition comprising a recombinant adeno-associated virus (rAAV) comprising a ZBTB7A-targeting inhibitory RNA-encoding DNA. In some embodiments, the psychiatric disorder is one of Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition. In some embodiments, the ZBTB7A-targeting inhibitory RNA-encoding DNA comprises a sequence selected from the group consisting of SEQ ID NO: 3; SEQ ID NO: 4; SEQ ID NO: 5; SEQ ID NO: 6; SEQ ID NO: 7; SEQ ID NO: 8; SEQ ID NO: 9; or SEQ ID NO: 10. In some embodiments, the recombinant AAV is administered by parenteral, intravenous, intrathecal, introcerebroventricular, or cisterna magna administration. In some embodiments, the intrathecal administration is by lumbar puncture.

In another aspect, provided herein is a recombinant adeno-associated virus including a ZBTB7A-targeting inhibitory RNA-encoding DNA including a sequence selected from the group consisting of SEQ ID NO: 3; SEQ ID NO: 4; SEQ ID NO: 5; SEQ ID NO: 6; SEQ ID NO: 7; SEQ ID NO: 8; SEQ ID NO: 9; or SEQ ID NO: 10. In another aspect, provided herein is a composition including the recombinant adeno-associated virus and a pharmaceutically acceptable carrier. In another aspect, provided herein are methods of inhibiting expression of ZBTB7A in a cell, including contacting the cell with the recombinant AAV, wherein the expression of the ZBTB7A-targeting inhibitory RNA inhibits expression of ZBTB7A in the cell. In some embodiments, the cell is in the orbitofrontal cortex (OFC) of a subject. In some embodiments, the cell is an astrocyte cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a heatmap of clustering of major depressive disorder (MDD) case and control samples at 1412 differentially expressed (DE) genes (rows, FDR<0.1).

FIG. 1B is a plot of the co-expression modules identified by weighted gene correlation network analysis (WGCNA) [top] and a heatmap of co-expression module correlation with MDD trait. * indicates adjusted P<0.05 significance of correlation.

FIG. 1C is a plot of Gene Ontology (GO) analysis for genes in significant co-expression modules.

FIG. 1D is a Venn diagram of shared and distinct open chromatin between neuronal and non-neuronal samples. Numbers indicate megabases of OCRs, “J” indicates the Jaccard index.

FIG. 1E is a plot of proportions of all and differential open chromatin regions (OCRs) stratified by genomic context.

FIG. 1F is a plot of overlap of all and differential OCRs with a reference study of lineage-specific brain open chromatin atlas.

FIG. 1G is a plot of enrichment of common genetic variants in MDD with all and differential OCRs when assayed by LD-score regression. Sets of OCRs were further stratified by genomic context to “Promoter OCRs” overlapping the 3 kb window around the transcription start site (TSS) and “Enhancer OCRs”.

FIG. 1H is a heatmap of clustering of MDD case and control non-neuronal samples at 203 differentially accessible OCRs (rows).

FIG. 1I is a plot of overlap between gene sets representing biological processes and pathways with the set of 203 differentially accessible OCRs between MDD cases and control. Top 15 enriched pathways are shown (BH-adjusted p-value<0.05). Dashed line indicates nominal significance. “GO”: gene ontology, “Re”: REACTOME.

FIG. 2A is a plot of the distribution of the discovered motif that is significantly enriched (e-value=1.9e-19) in MDD-specific OCRs.

FIG. 2B is a plot of GO biological processes (BP) terms from MEME-GoMo, based on gene targets of regulatory regions containing the discovered motif. Top 10 most significant terms are shown (BH-adjusted p-value<0.05). Dashed line indicates p=0.05 significance.

FIG. 2C is a plot of the correlation coefficients for transcription factor (TF) candidate recognition motifs against discovered motif (x-axis), and percent alignment between TF candidate recognition motifs with discovered motif (y-axis and color key).

FIG. 2D is a plot of percent expression of TF candidate genes (CT value) over reference gene (HPRT1). “n.d.” indicates not detected.

FIG. 2E is a plot of normalized fold change of ZBTB7A transcripts in bulk OFC postmortem human tissues from MDD (n=20) vs. control (n=19) samples. Student's two-tailed t-test [t37=3.215, ** p=0.0027].

FIG. 2F is a plot of normalized fold change of ZBTB7A protein in bulk OFC postmortem human tissues from MDD (n=15) vs. control (n=12) samples. Student's two-tailed t-test [t25=2.441, *p=0.0221].

FIG. 2G is a plot of aggregated footprint scores across ZBTB7A transcription factor binding sites that are bound in either MDD or control samples of neuronal or non-neuronal cells. Note that the effect of Tn5 transposase bias is not fully corrected, resulting into unsmoothed signal.

FIG. 2H is a series of representative pile-up traces of cell specific ATAC-seq signal overlapping PRR5L gene. Four OCRs, all being dysregulated between MDD cases and controls (p-value<0.05) in non-neuronal cells, are highlighted. The most significantly dysregulated OCR (FDR<0.05) overlaps two transcription factor binding sites of ZBTB7A.

FIG. 2I is a plot of GO analysis with CellMarker Augmented Database and CHEA ENCODE Consensus database for genes in non-neuronal specific promoters, filtered by logFC>1, (+/−) 3000 bp from TSS.

FIG. 2J is a plot of GO analysis with CellMarker Augmented Database and CHEA ENCODE Consensus database for genes in downregulated differentially expressed genes from human MDD RNA-seq.

FIG. 2K is a plot of social interaction ratio for control (n=8) vs. chronic stress: susceptible (n=11) vs. chronic stress: resilient mouse (n=9) groups. 1-way ANOVA [F2,25=66.99], followed by Tukey's MC test: control vs. stress susceptible **** p=<. 0001, stress susceptible vs. stress resilient **** p=<. 0001, control vs. stress resilient ns, p=0.151.

FIG. 2L is a plot of normalized fold change protein expression of Zbtb7a in mouse OFC bulk tissues collected from control vs. chronic stress: susceptible vs. chronic stress: resilient mouse groups. 1-way ANOVA [F2,24=4.883], followed by Tukey's MC test: control vs. stress susceptible *p=0.03, stress susceptible vs. stress resilient *p=0.039, control vs. stress resilient ns, p=0.979.

FIG. 2M is a plot of FKPM values for Zbtb7a in astrocyte specific CSDS TRAP-seq data set [GSE139684], with n=3 control, n=5 stress: susceptible, n=4 stress-resililent. 1-way ANOVA [F2,9=10.01], followed by Tukey's MC test: control vs. stress susceptible *p=0.012, stress susceptible vs. stress resilient *p=0.01, control vs. stress resilient ns, p=0.989. All data graphed as means±SEM.

FIG. 2N is a plot of normalized fold change Zbtb7a mRNA expression in MACs-isolated astrocytes from chronically stressed OFC mouse tissues vs. control (n=4/group). Two-tailed Student's t-test [t6=3.458]. * p=0.013.

FIG. 20 is a plot of normalized fold change Zbtb7a mRNA expression in MACs-isolated neurons from chronically stressed OFC mouse tissues vs. control (n=4/group). Two-tailed Student's t-test [t6=1.454]. ns, p=0.196.

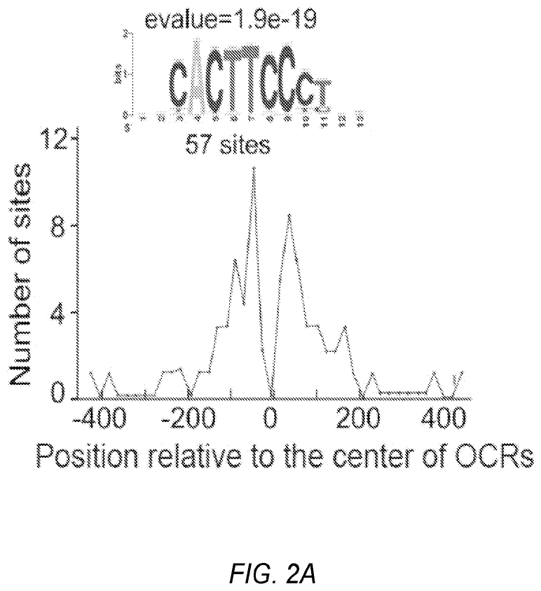

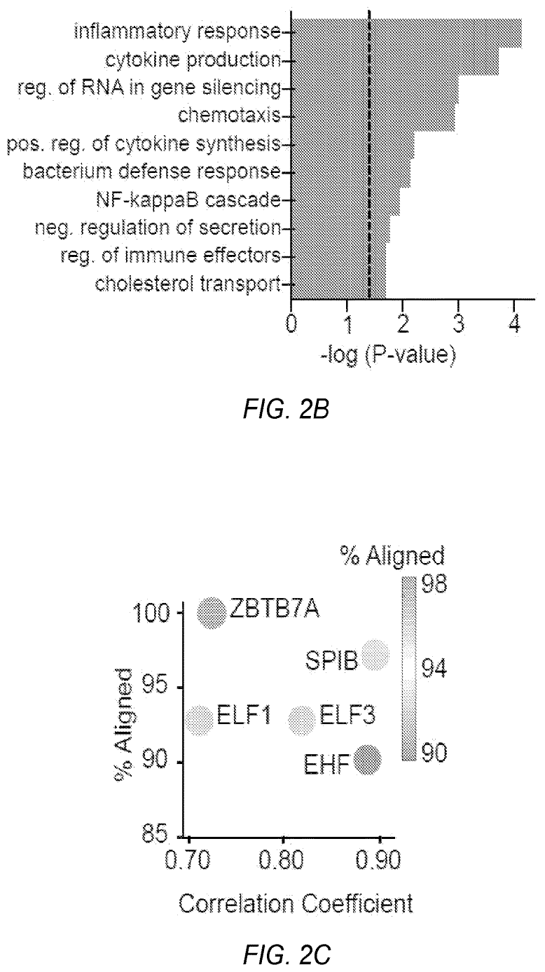

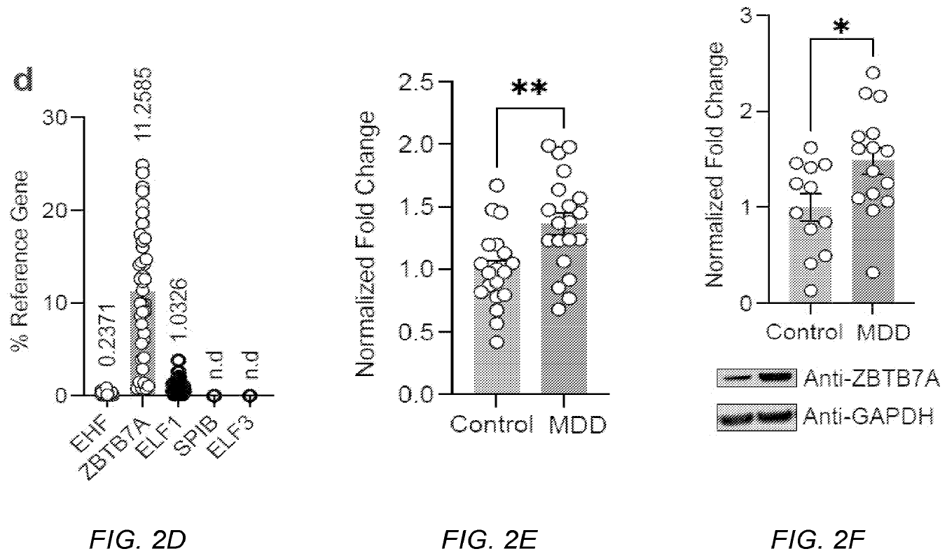

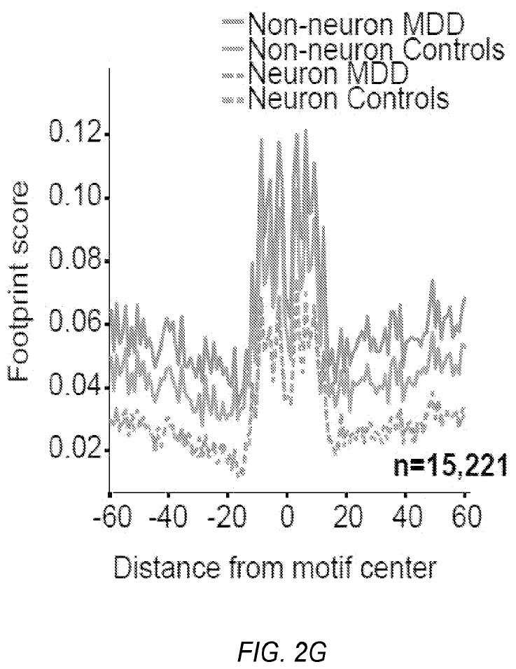

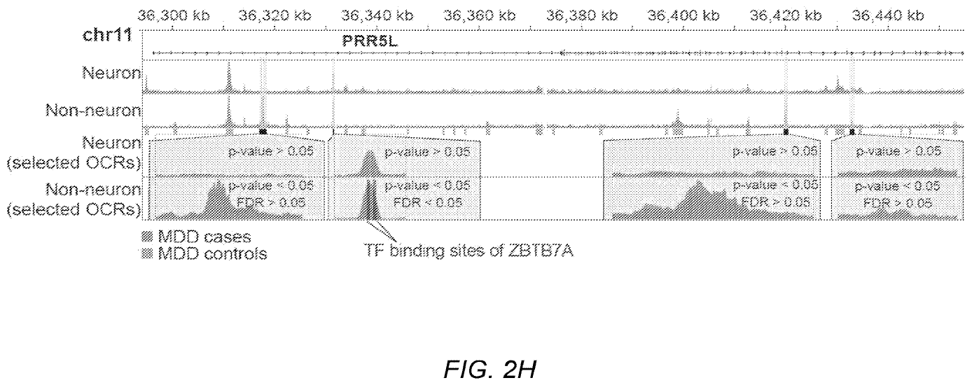

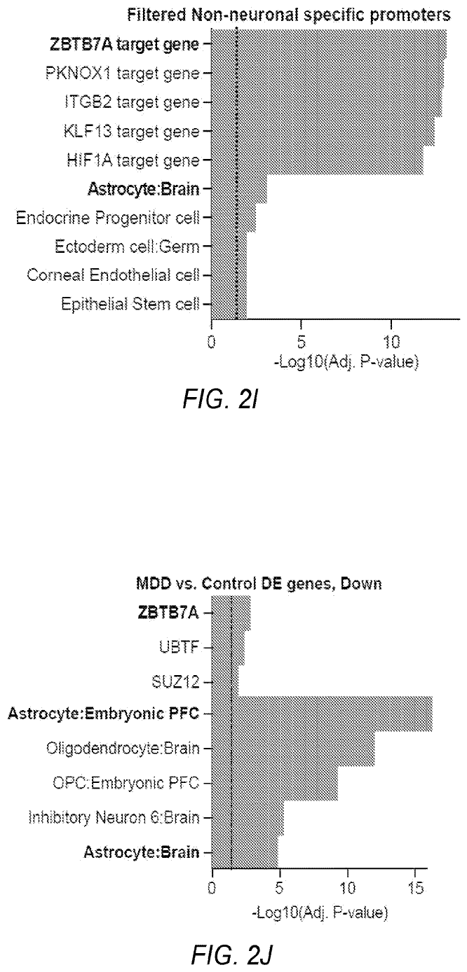

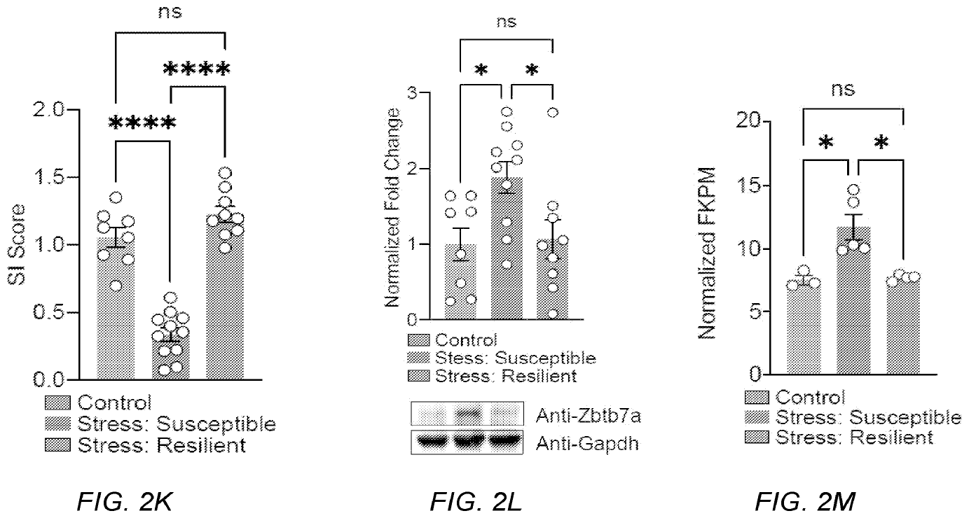

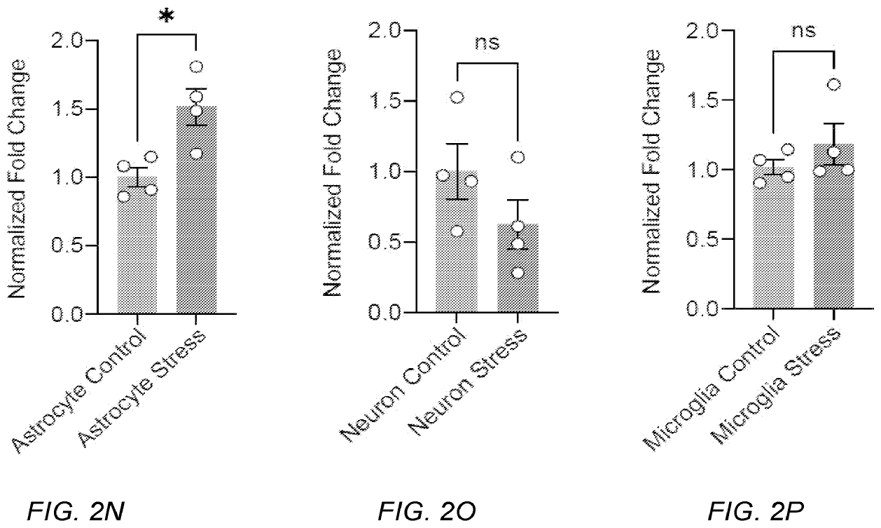

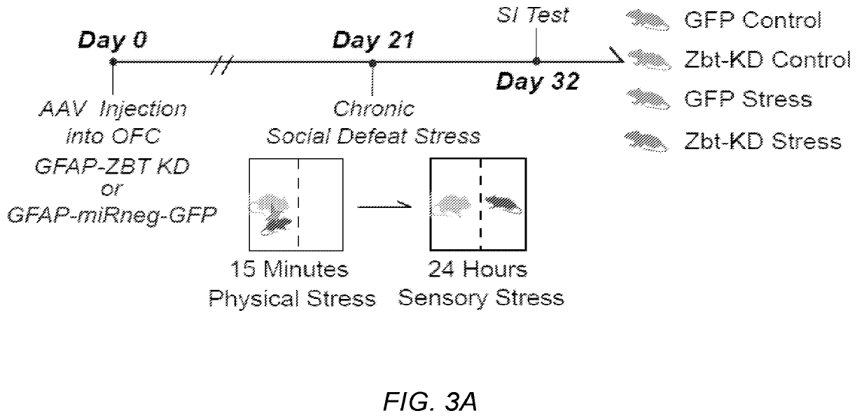

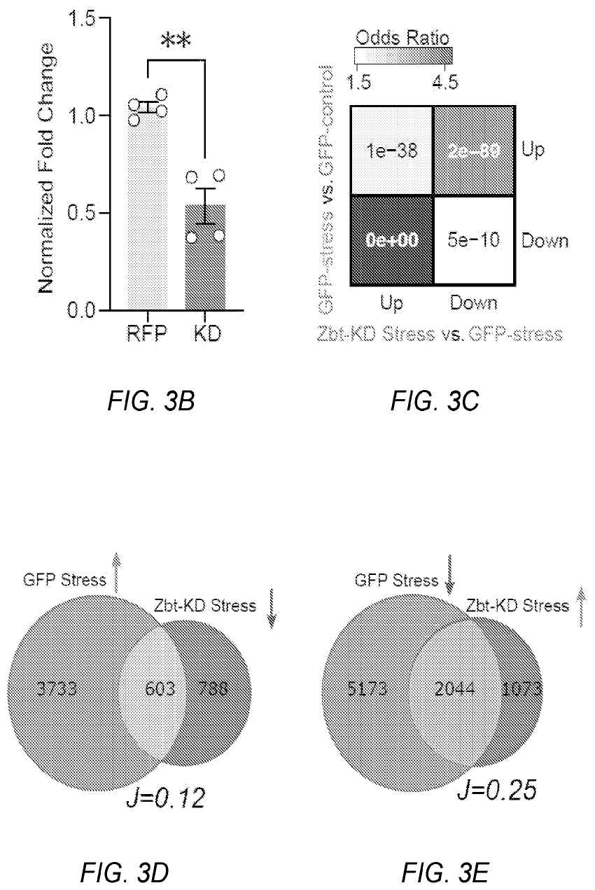

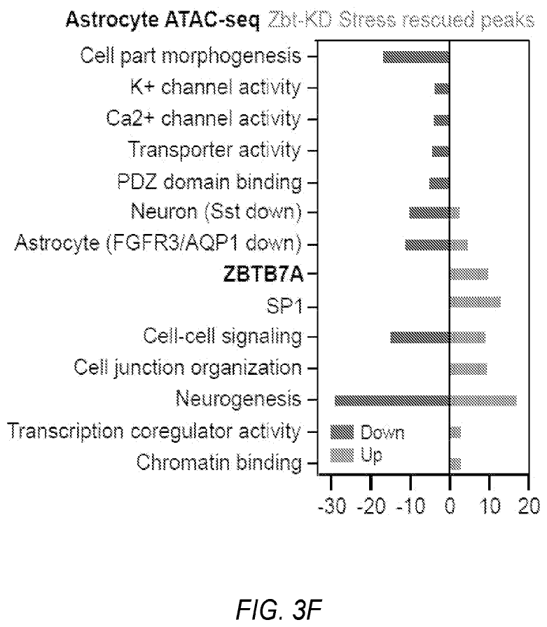

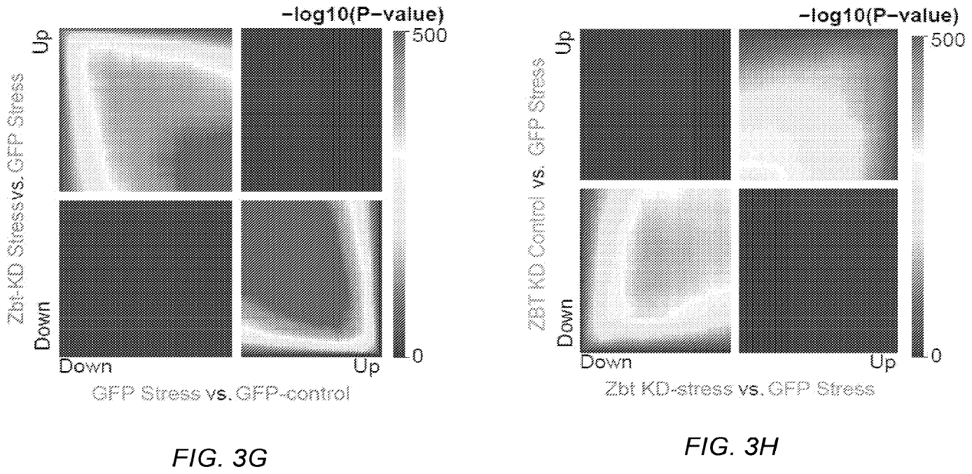

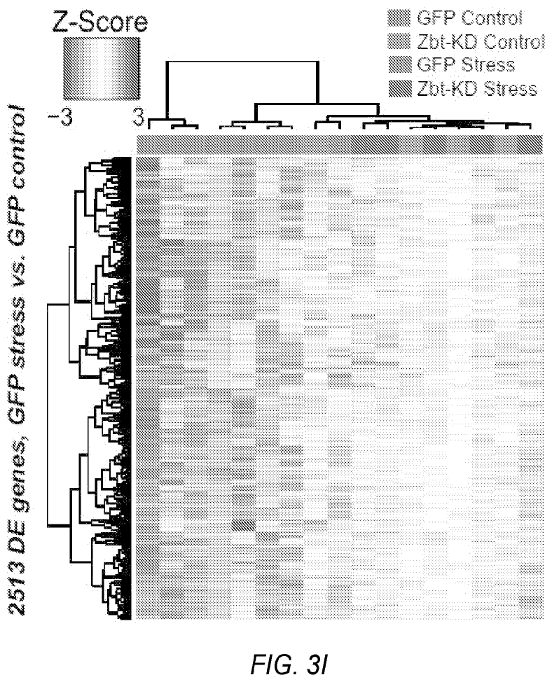

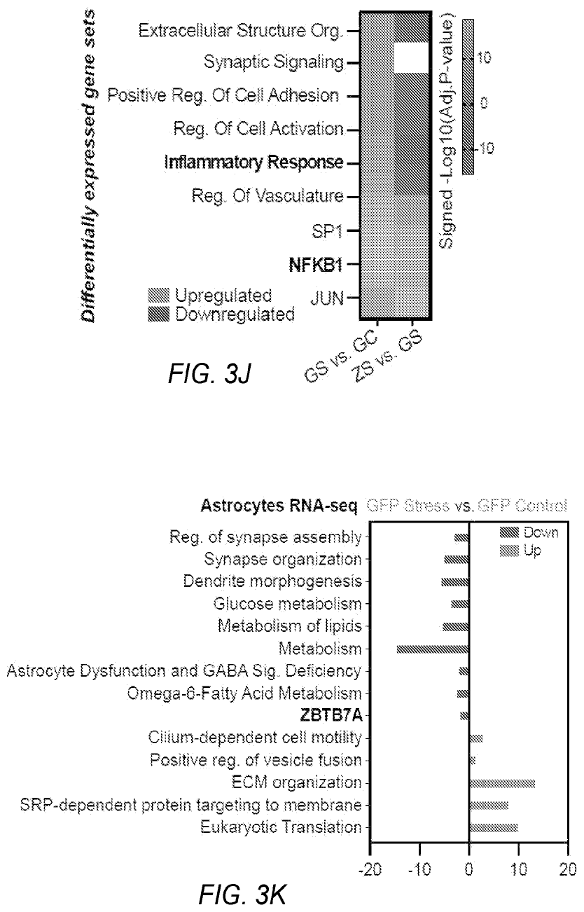

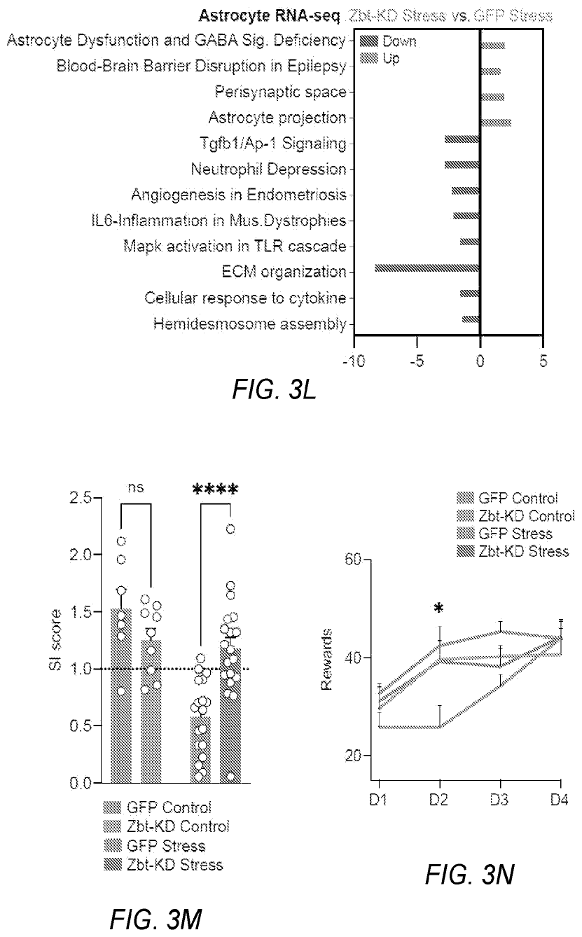

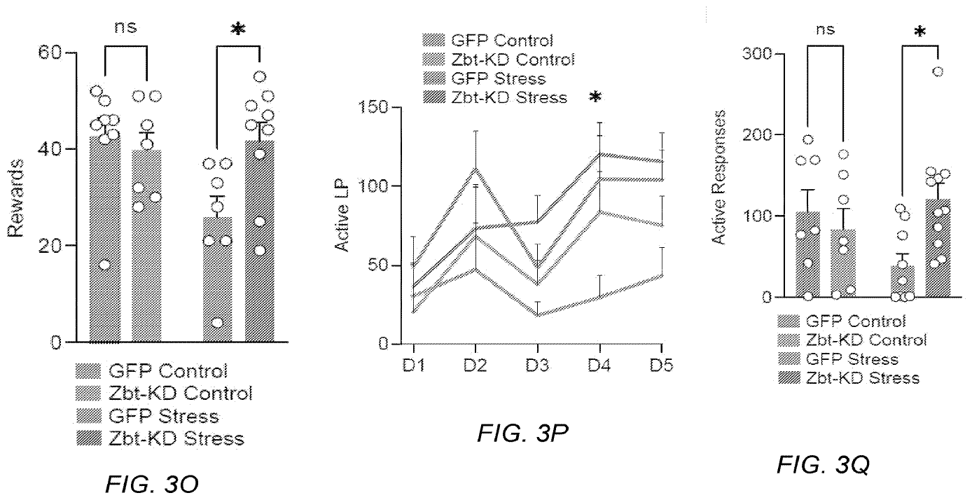

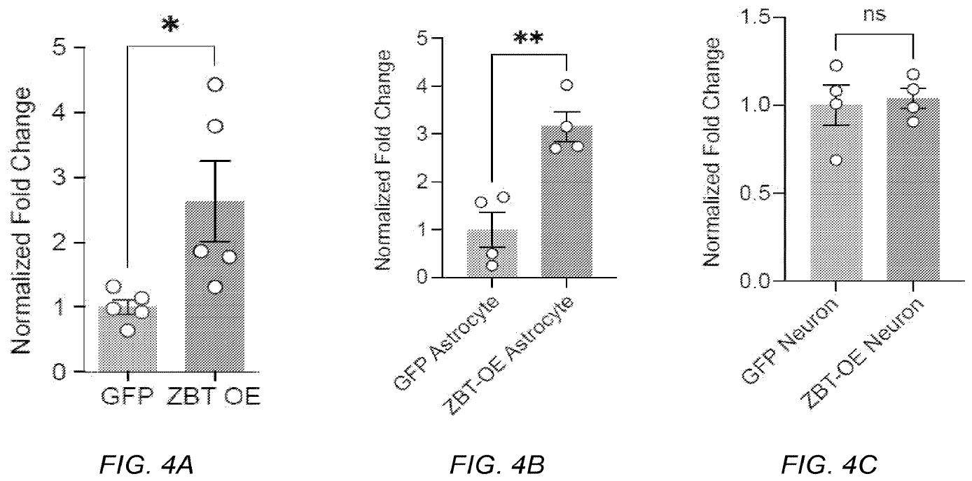

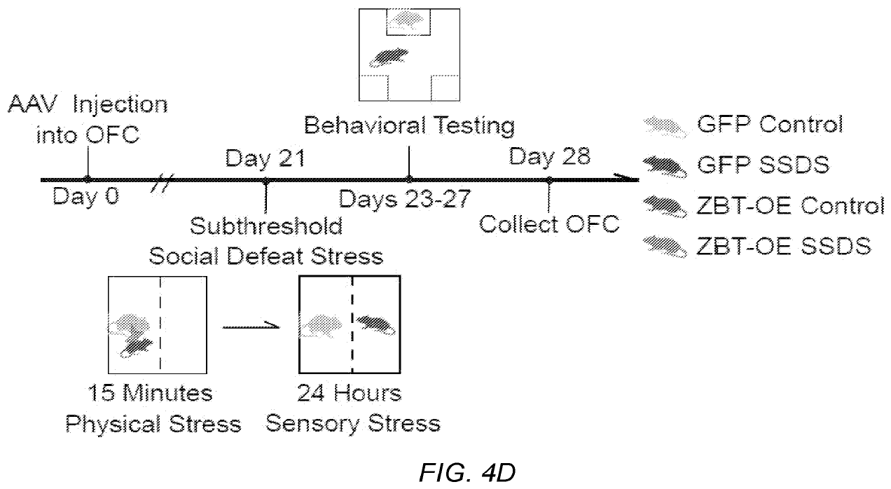

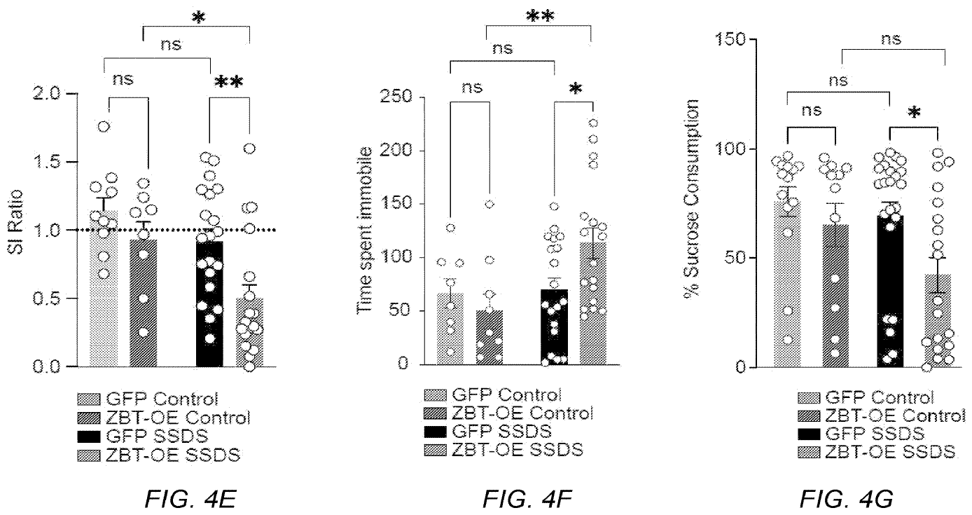

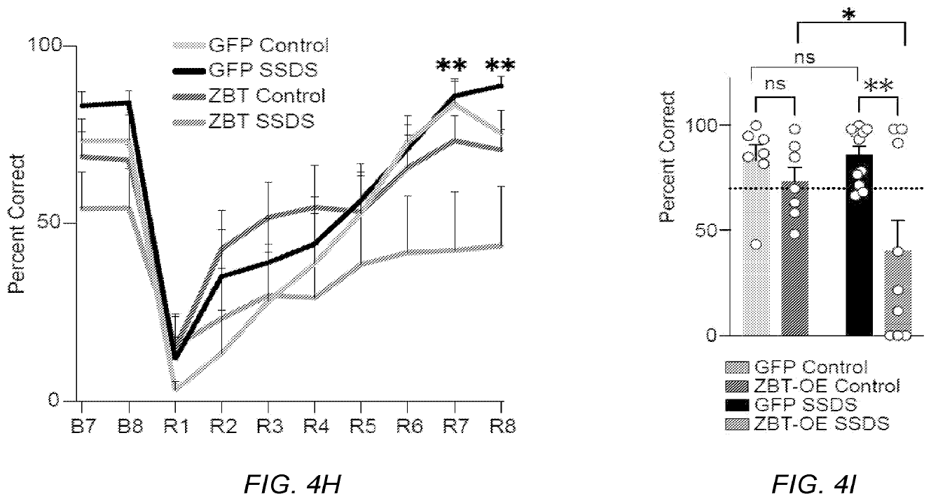

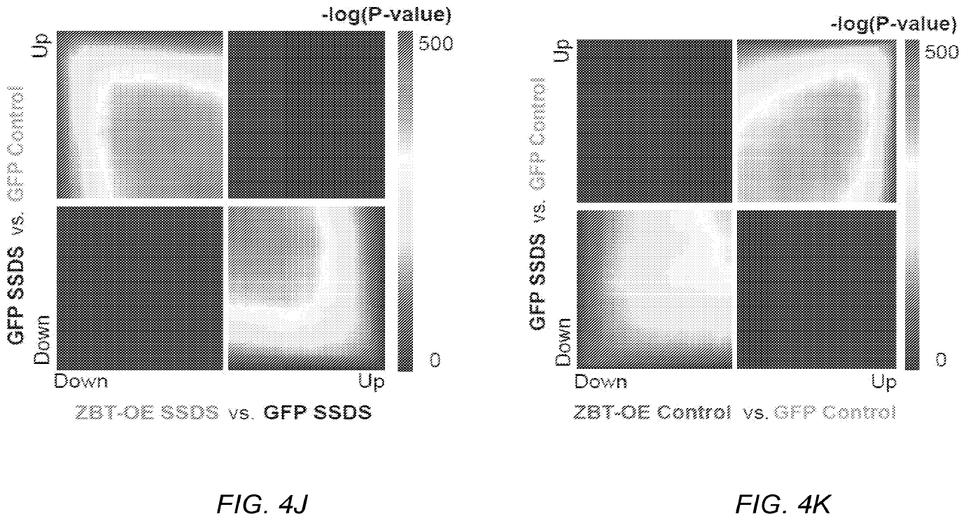

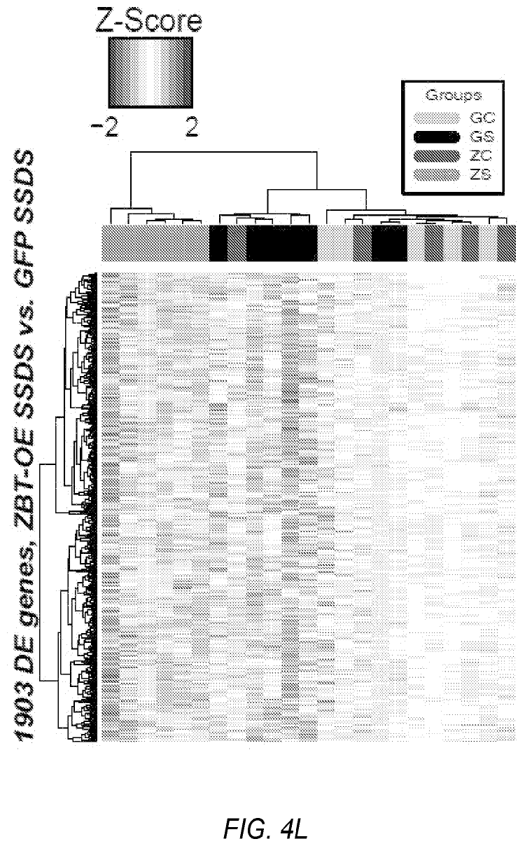

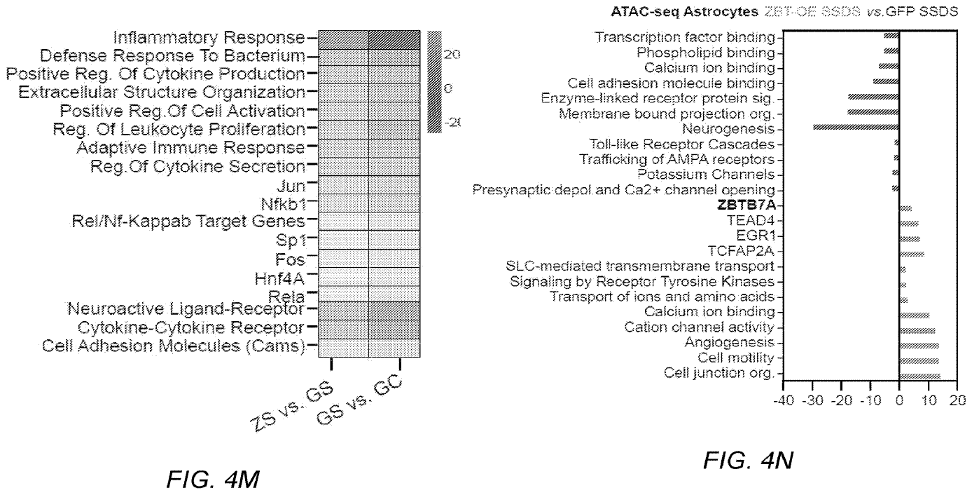

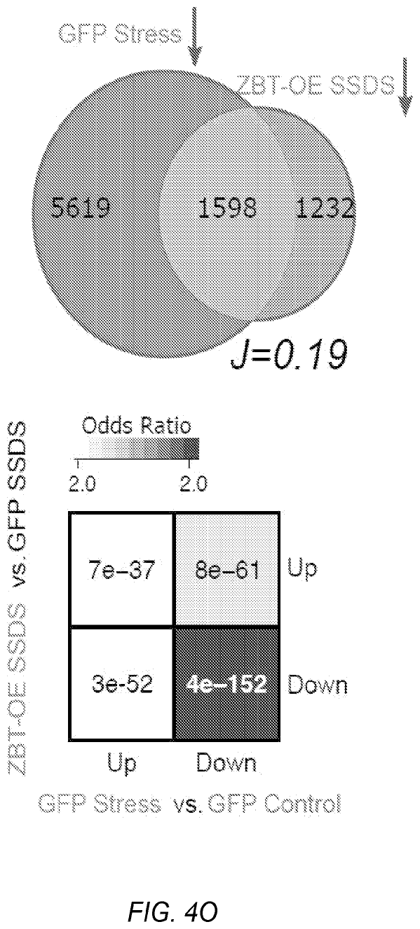

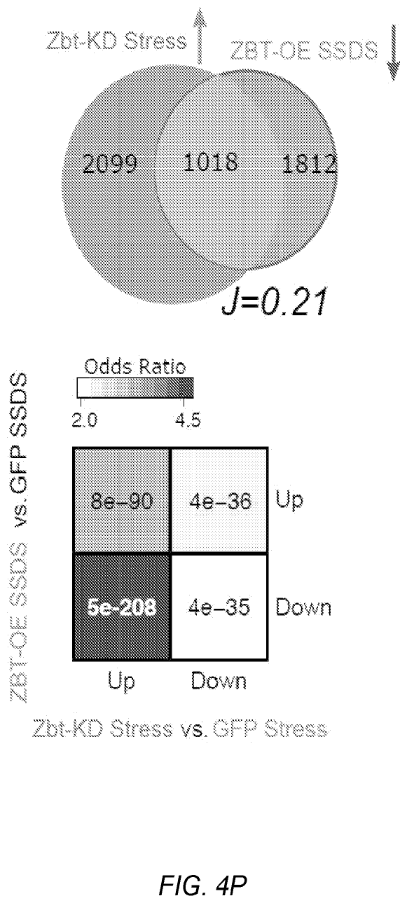

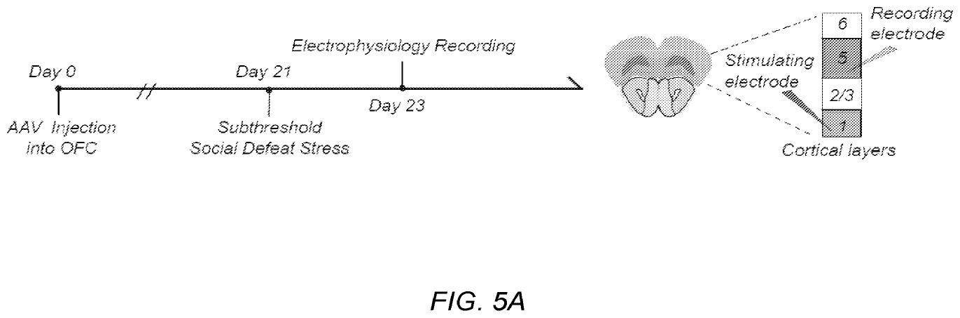

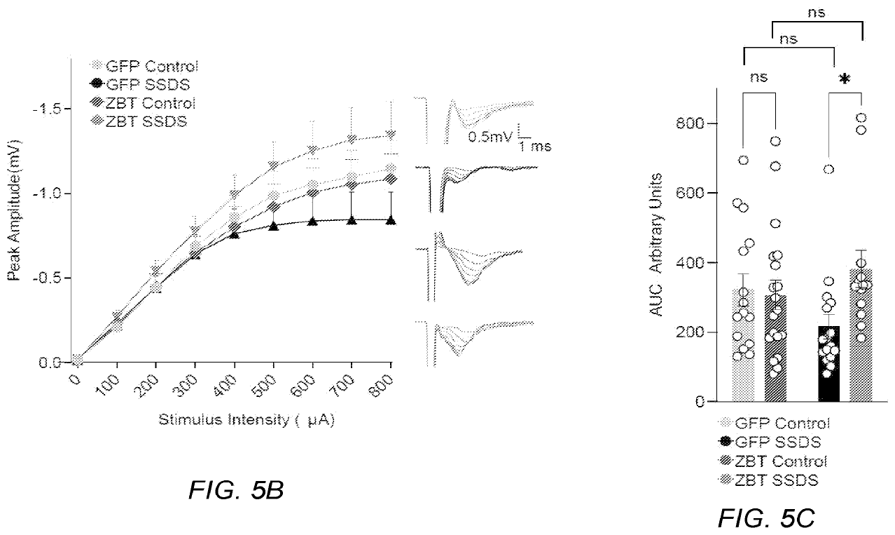

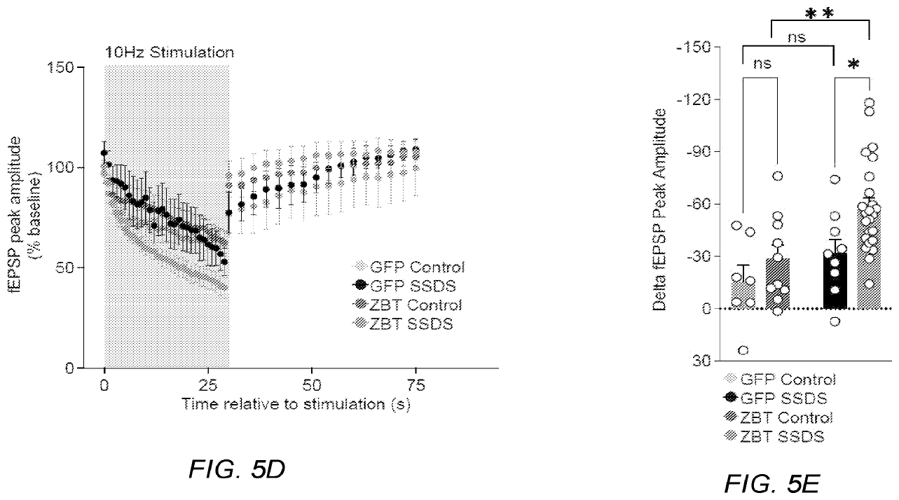

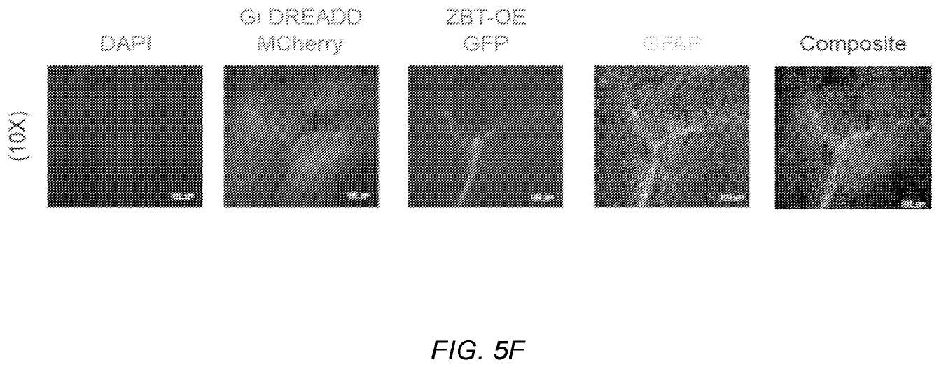

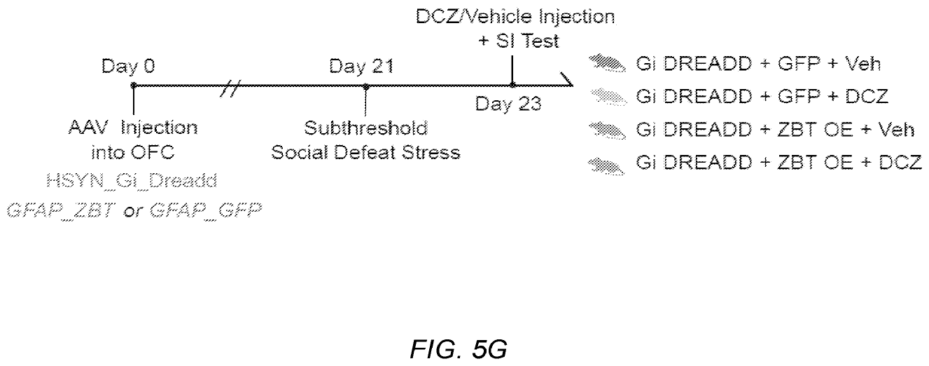

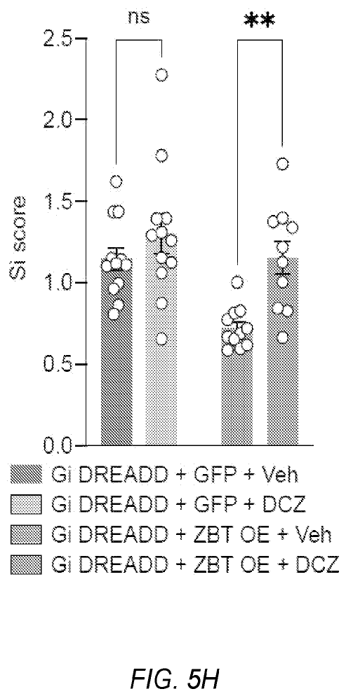

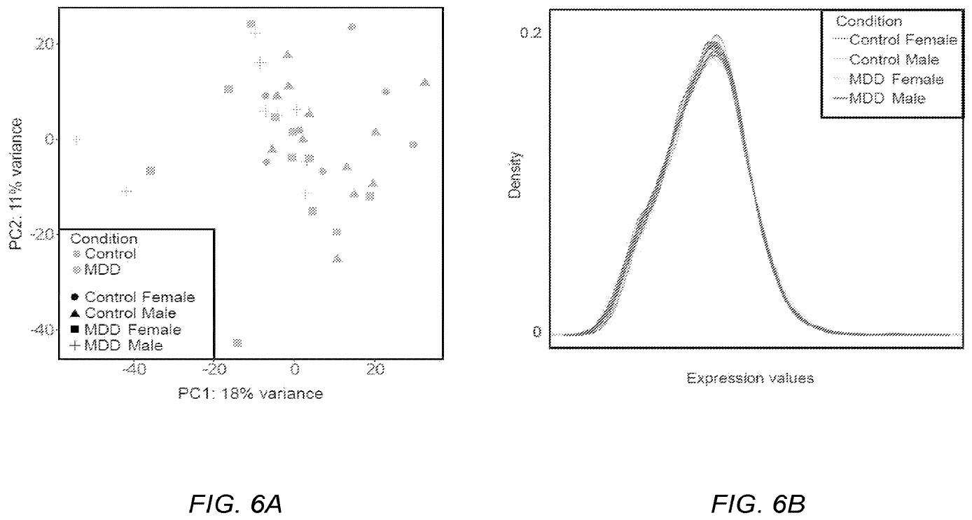

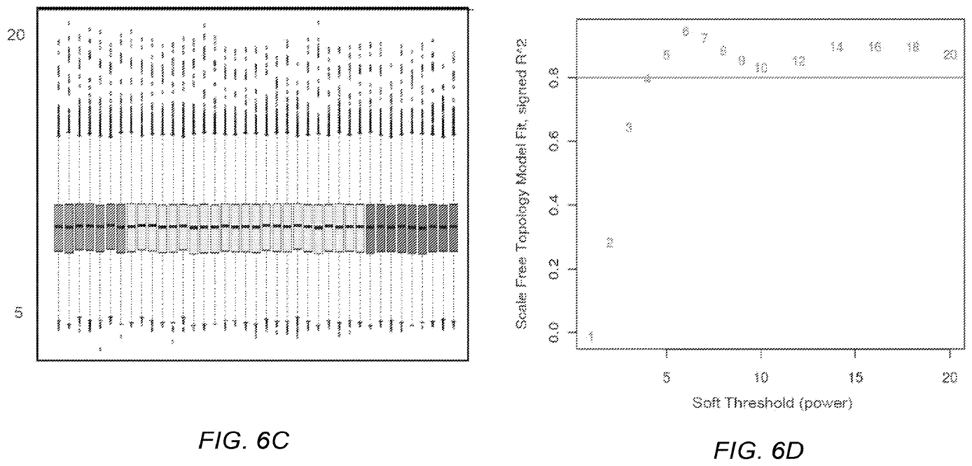

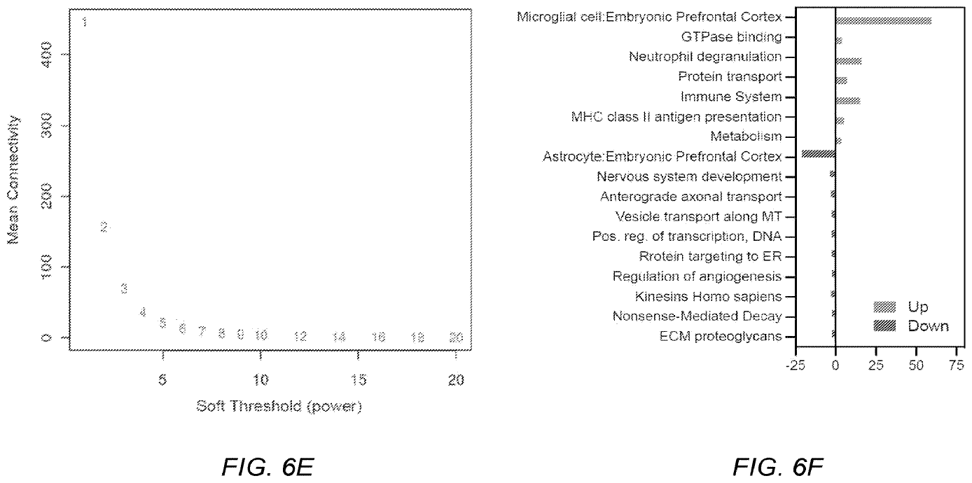

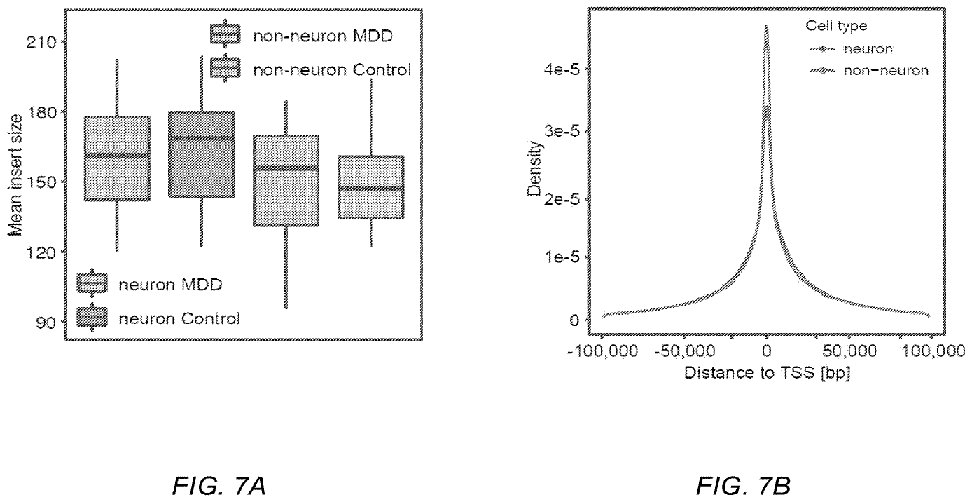

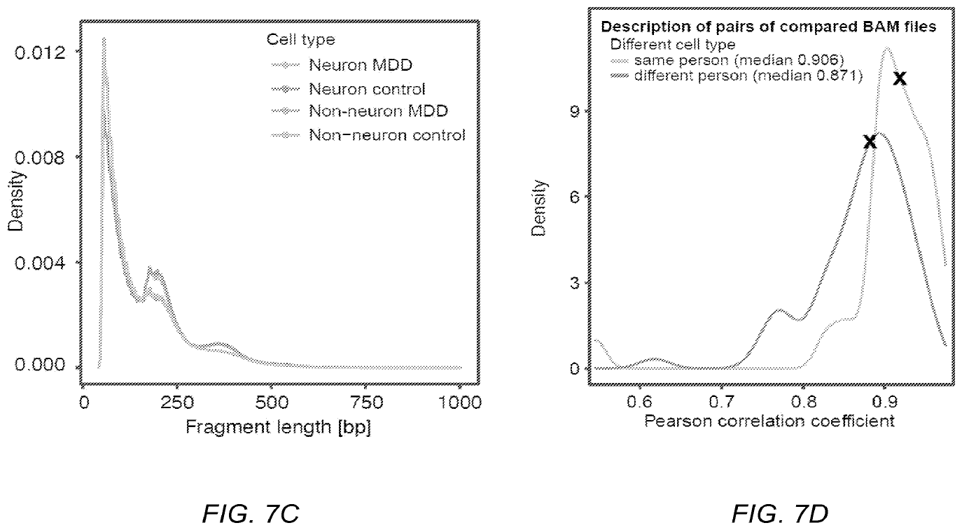

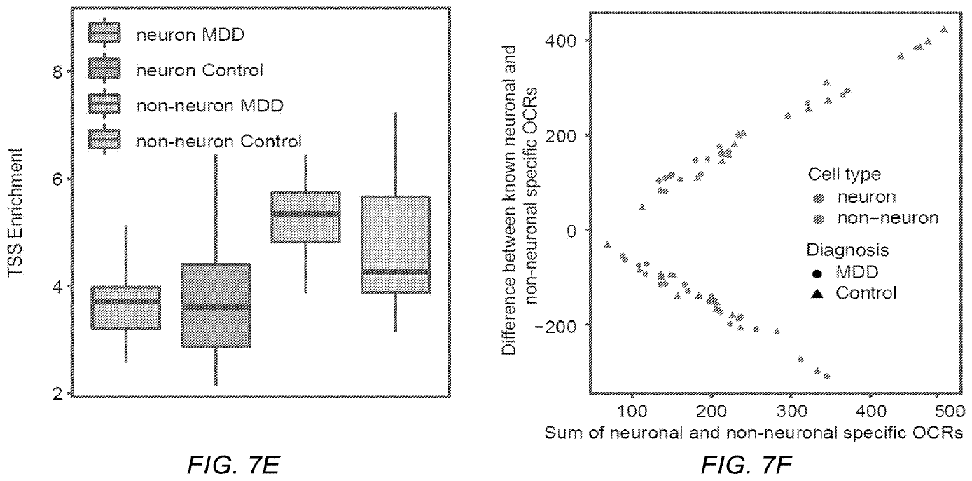

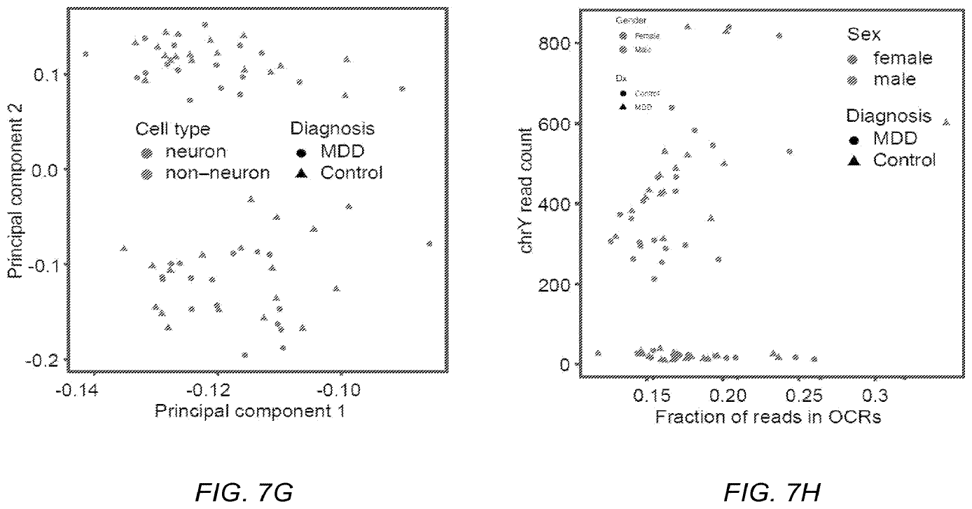

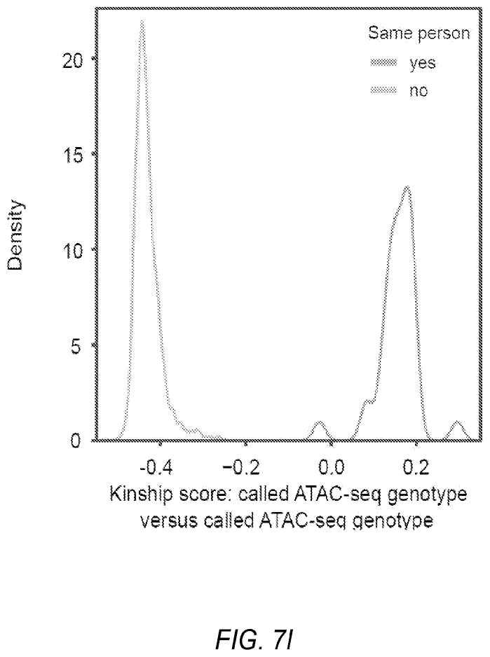

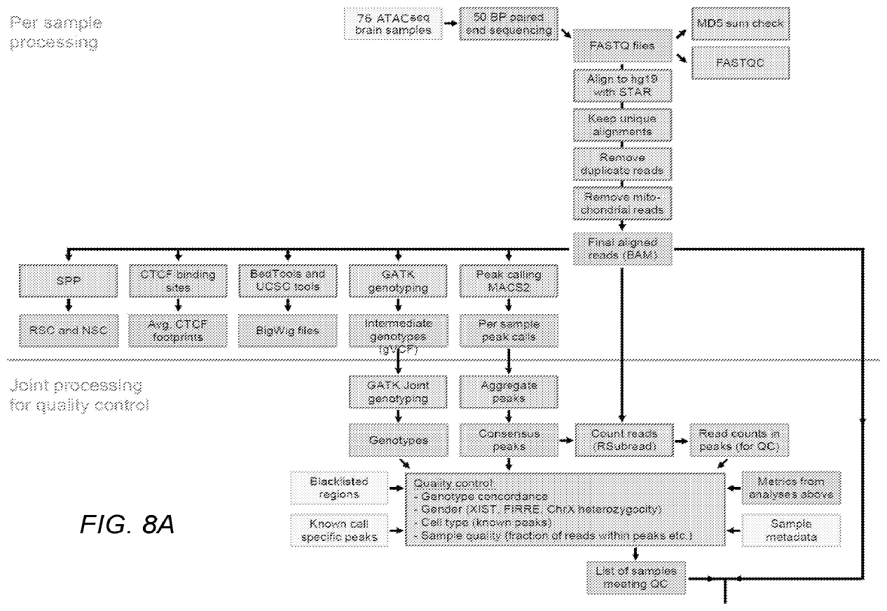

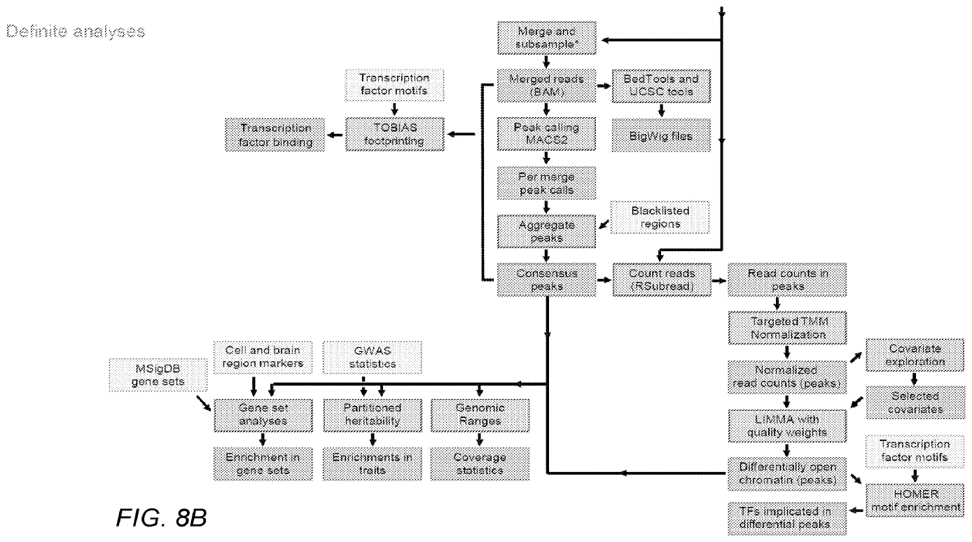

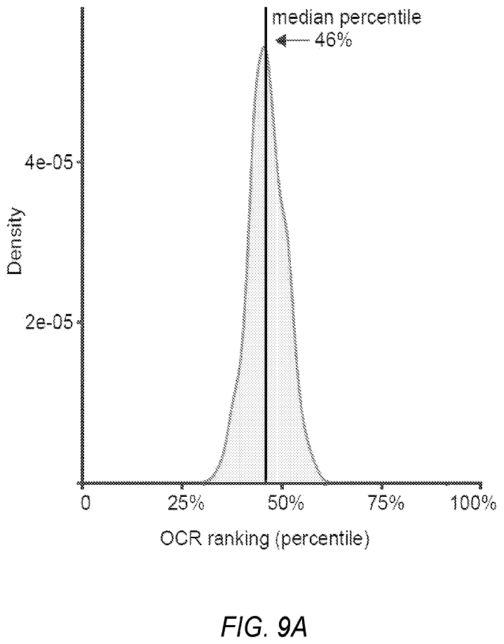

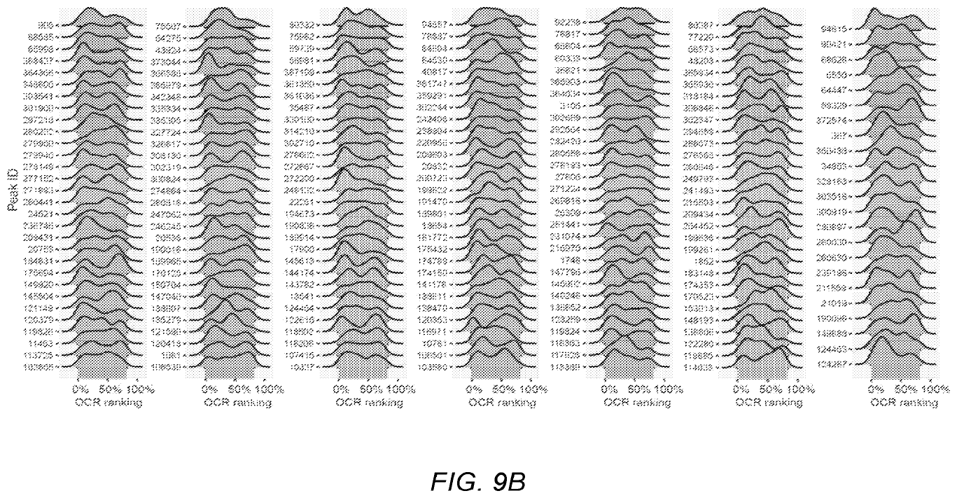

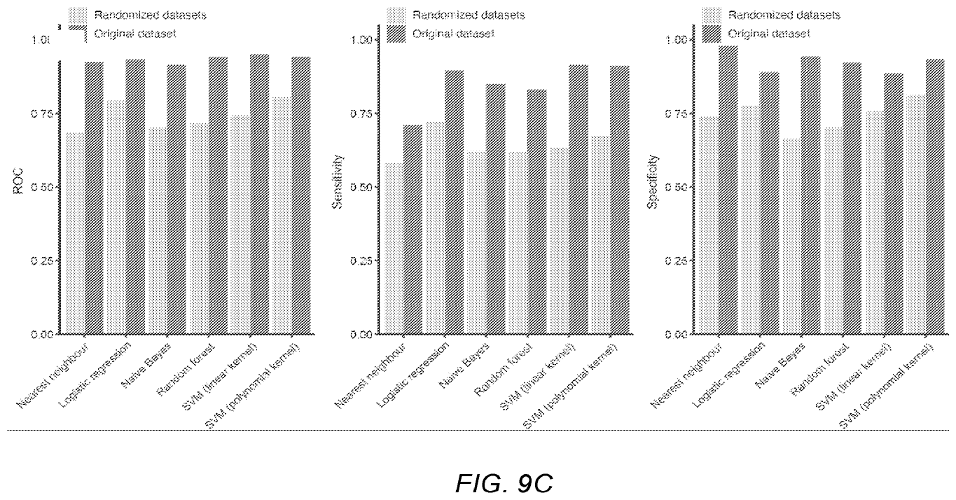

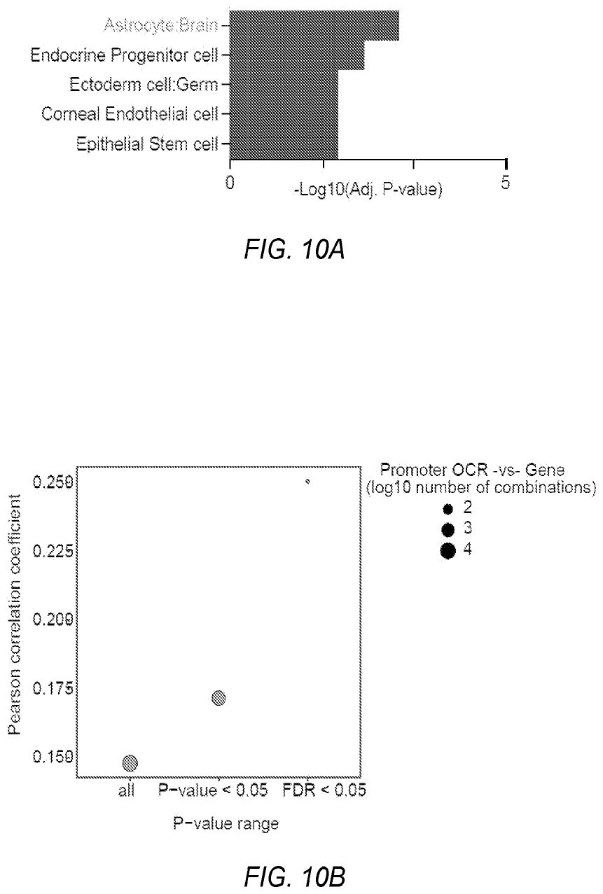

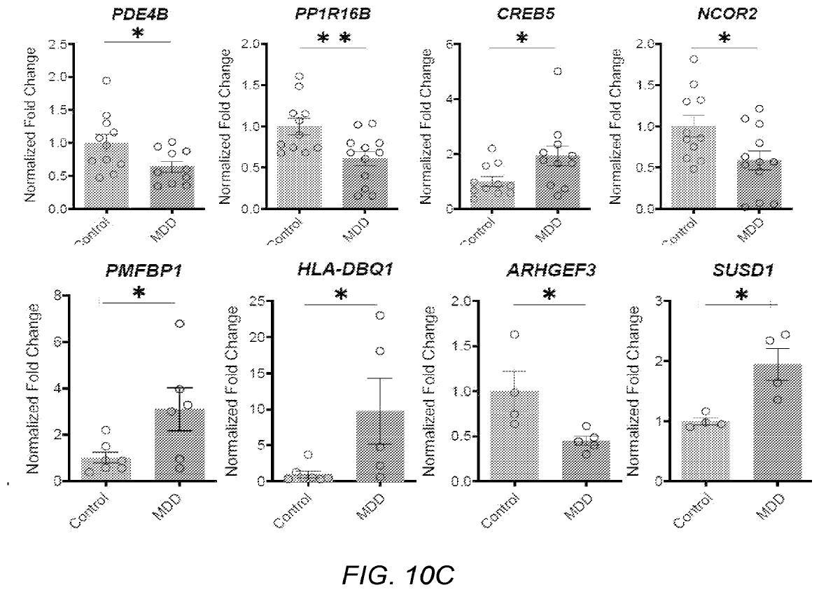

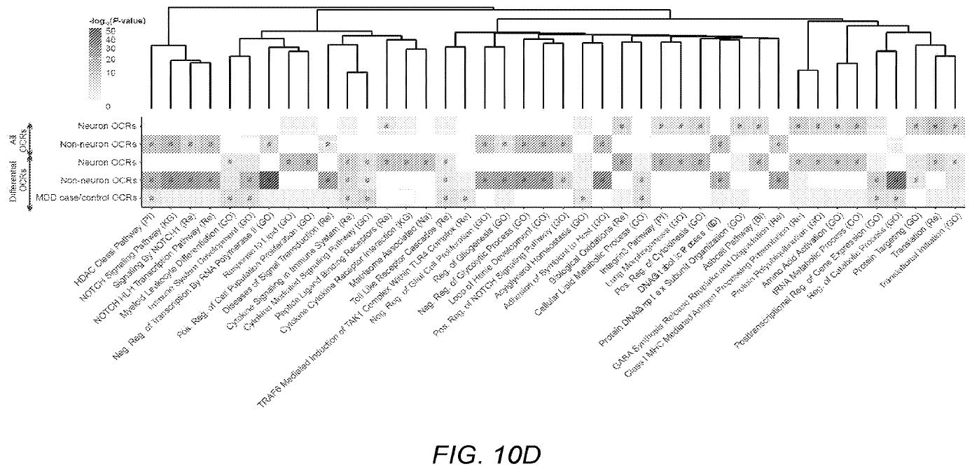

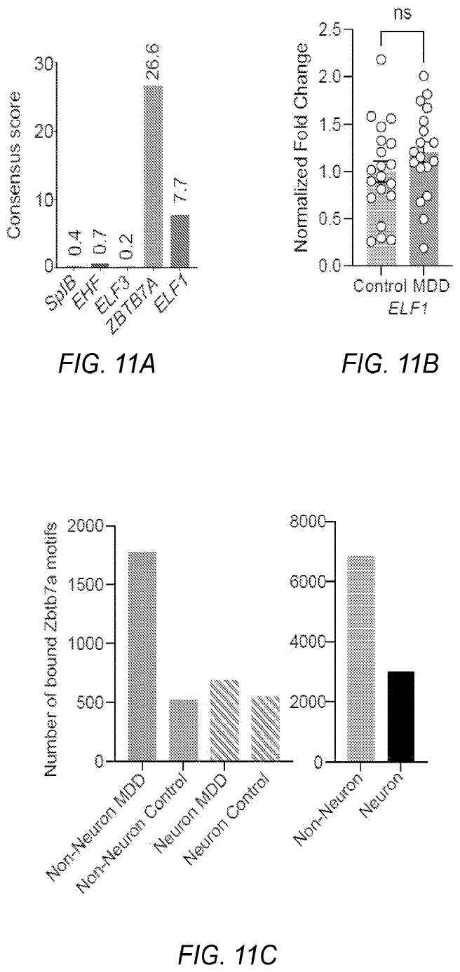

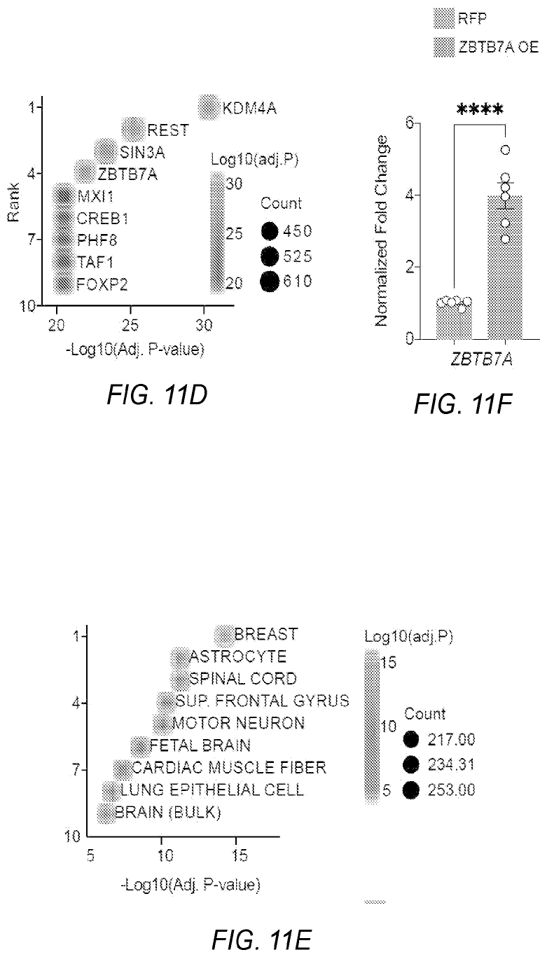

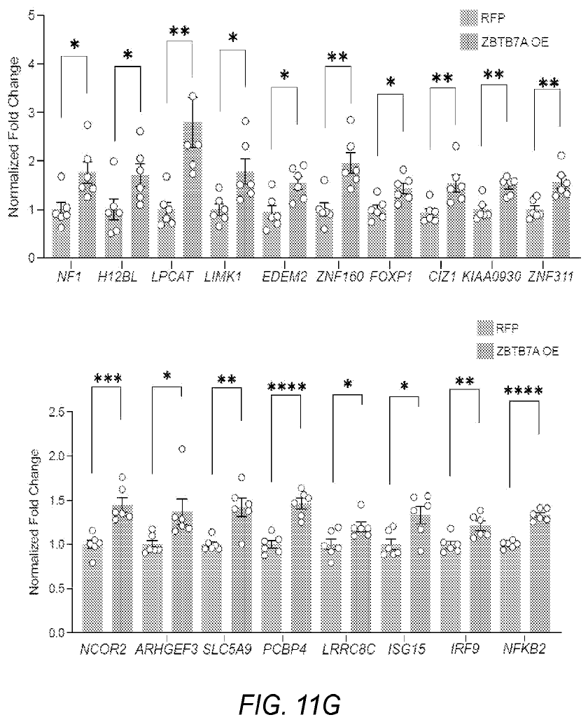

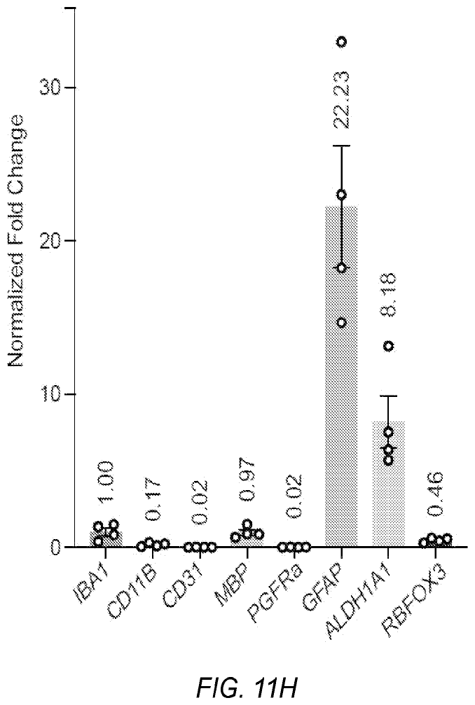

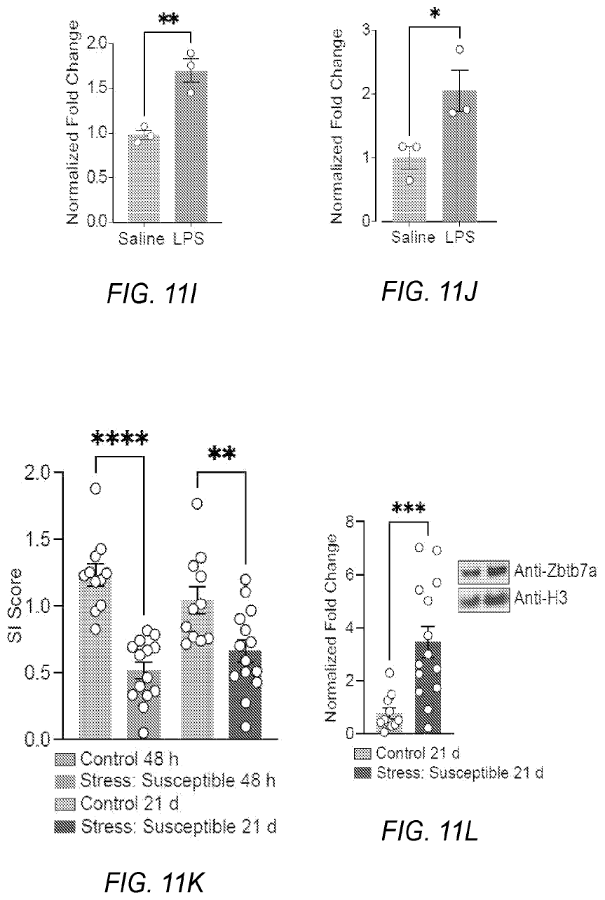

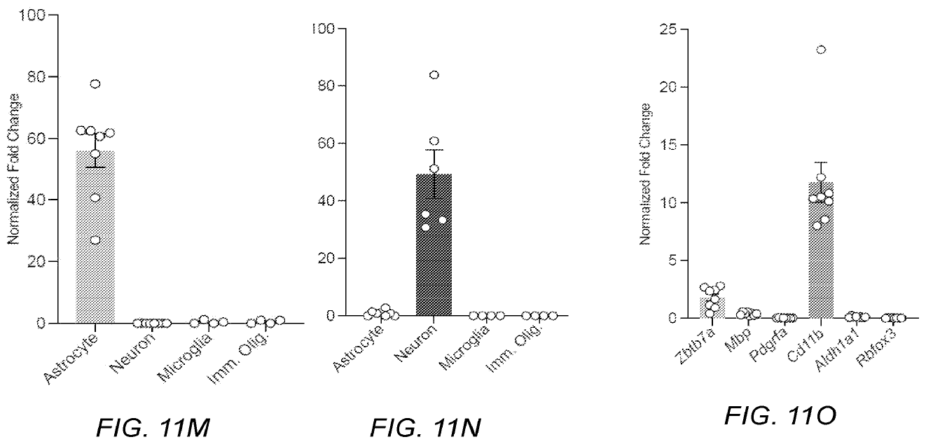

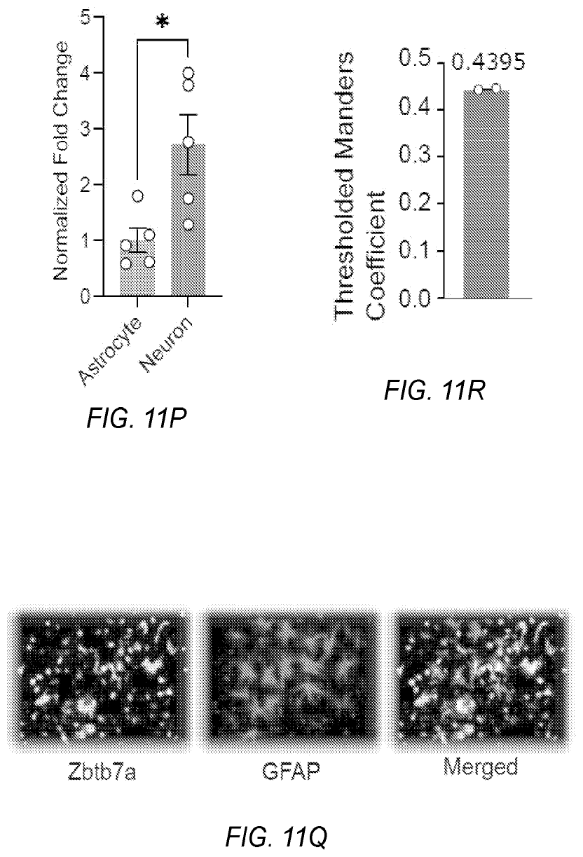

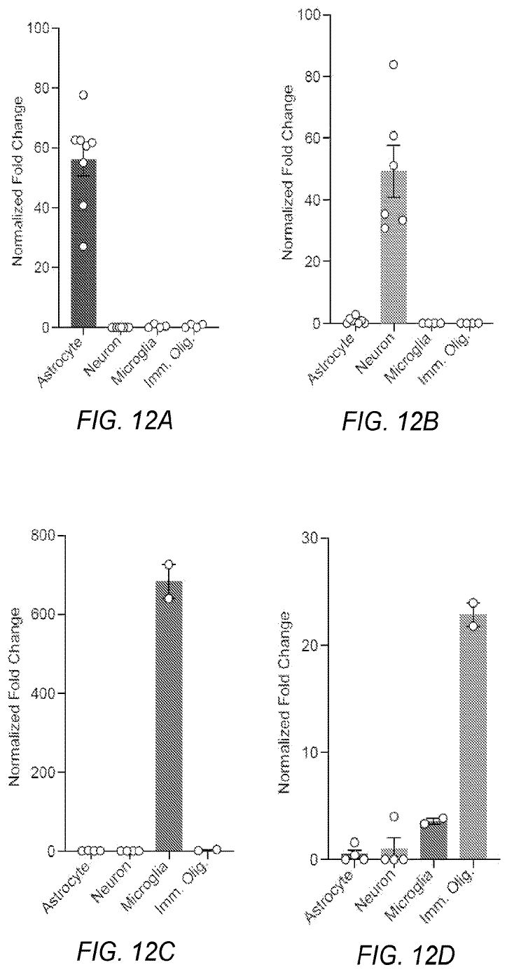

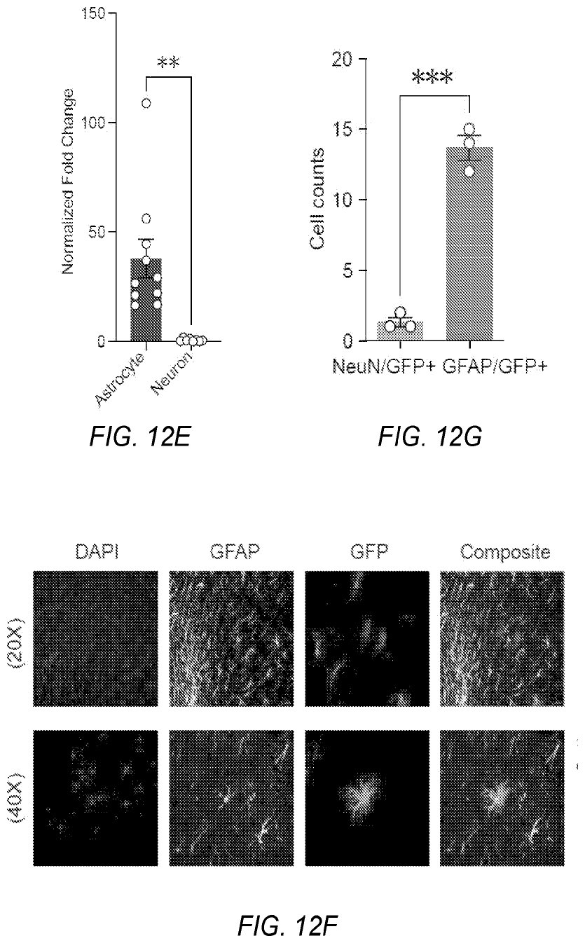

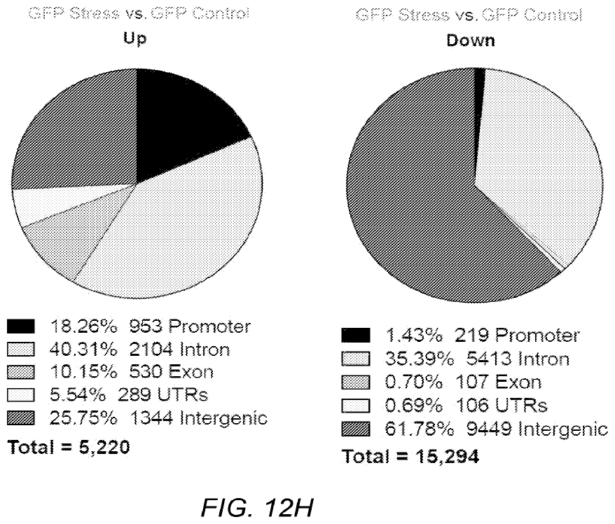

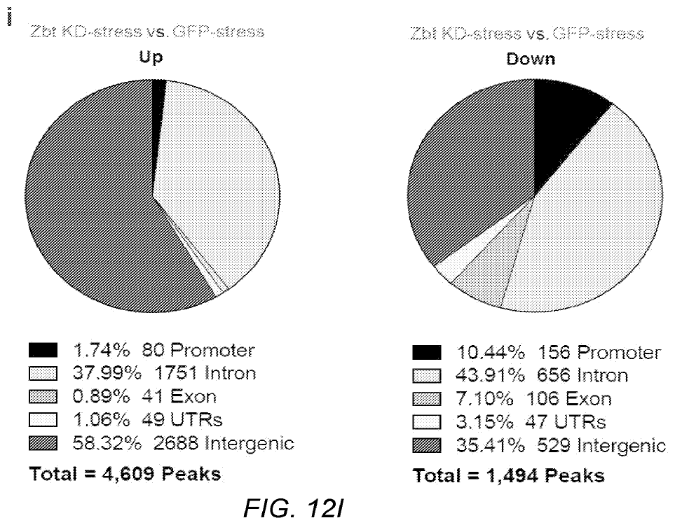

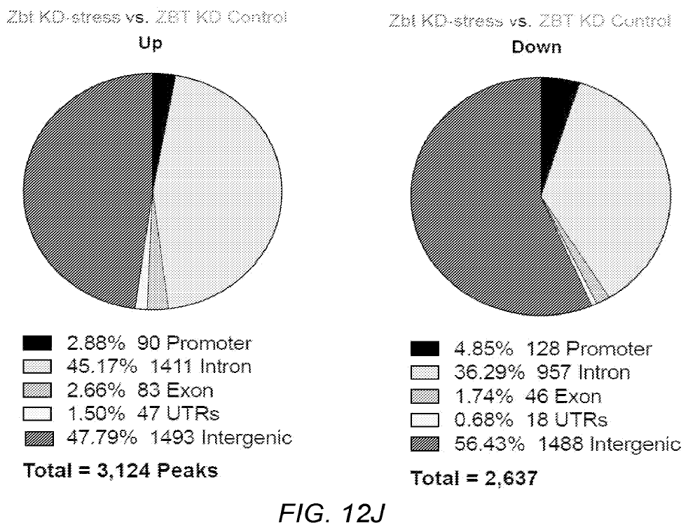

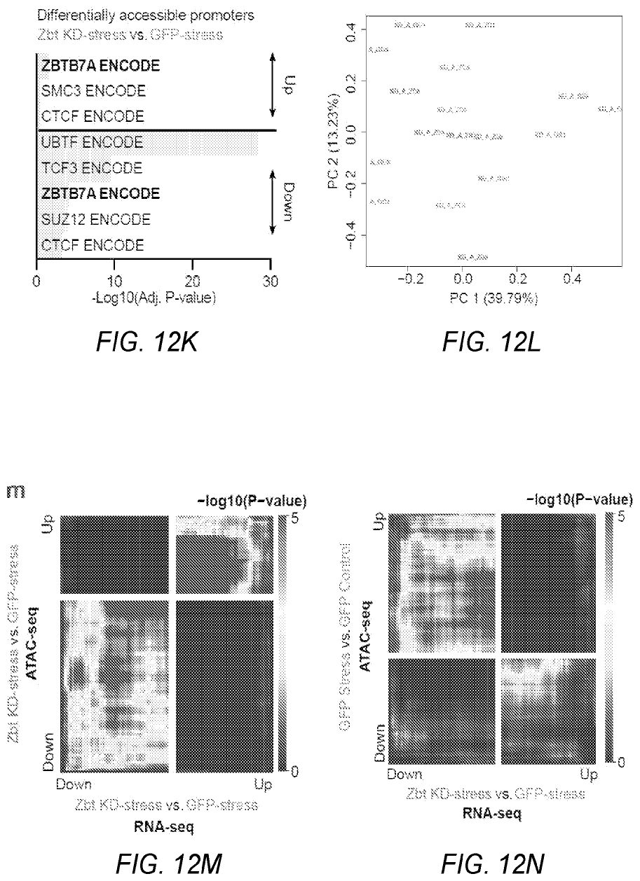

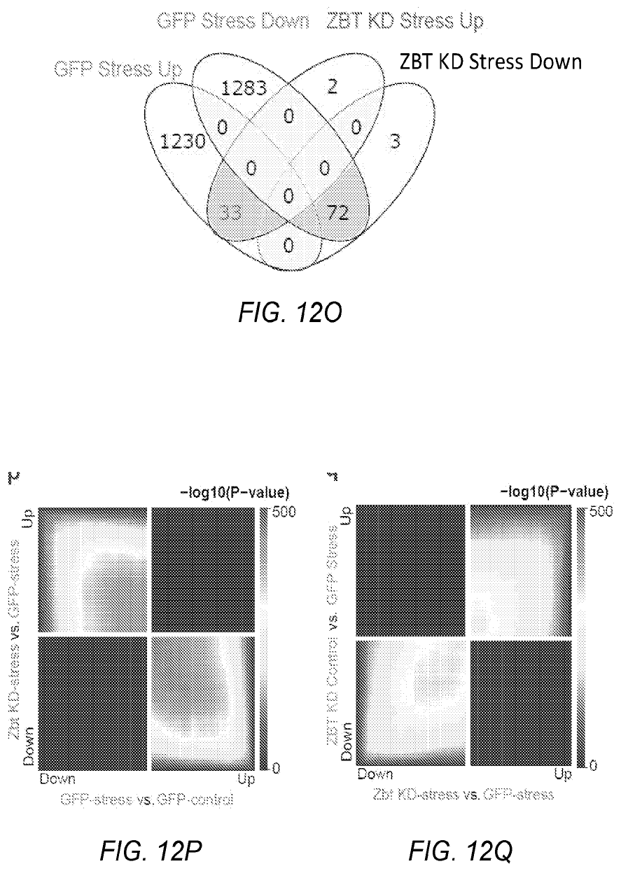

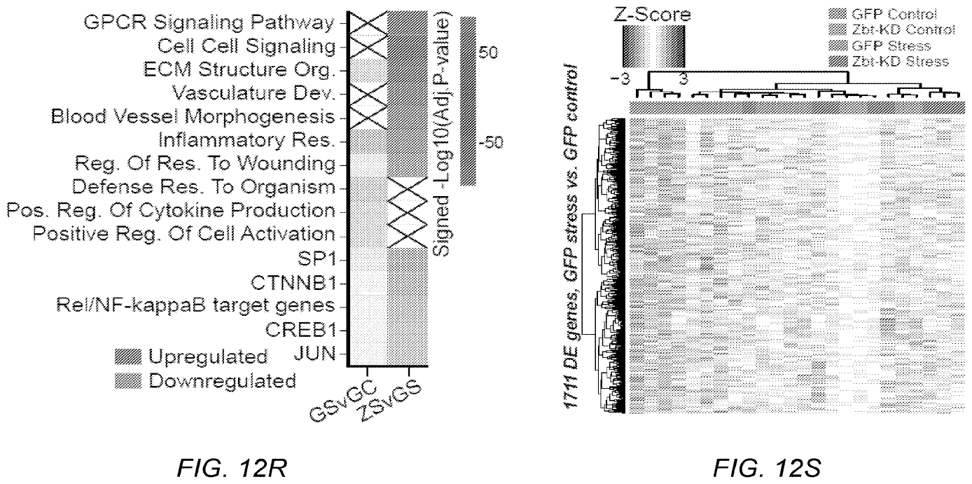

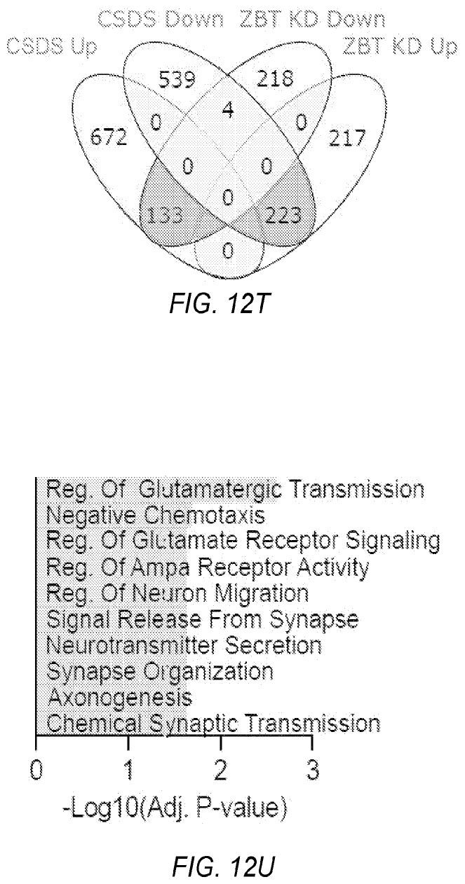

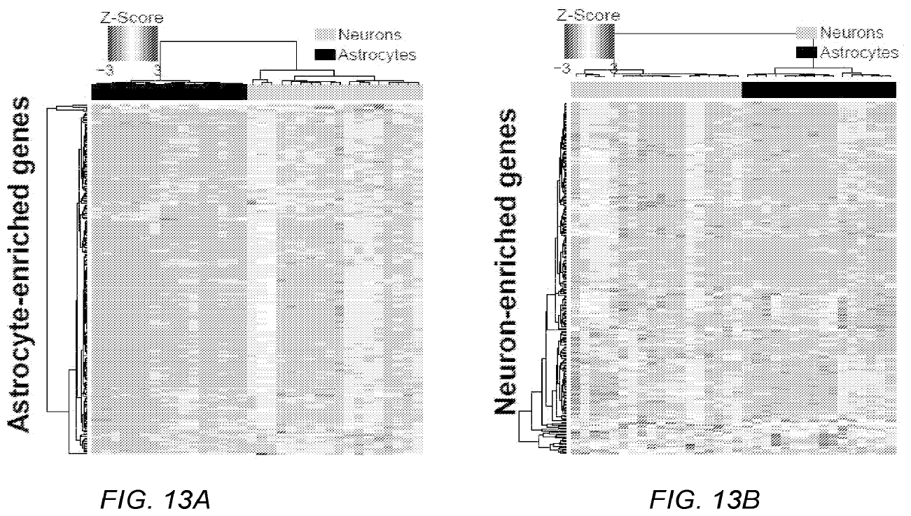

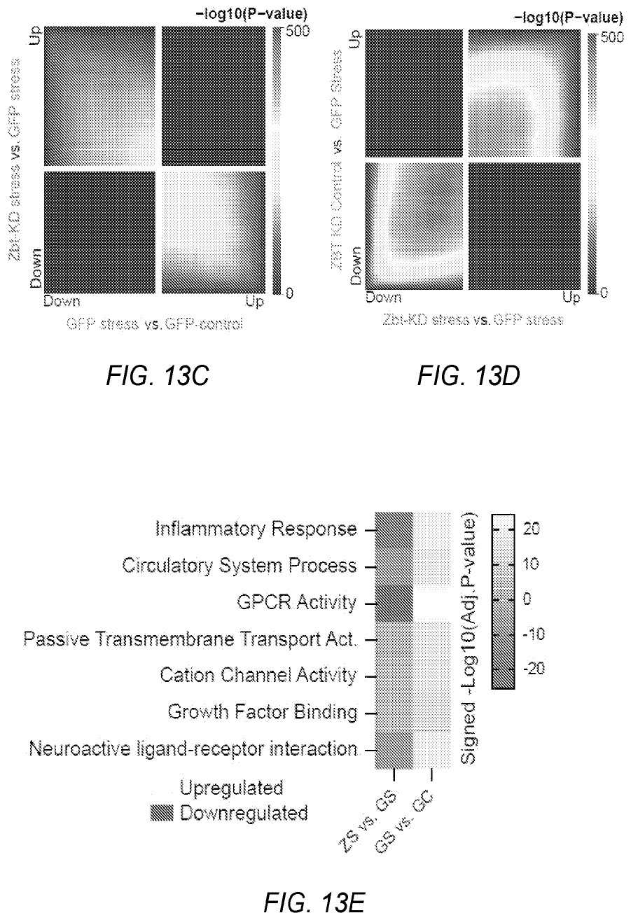

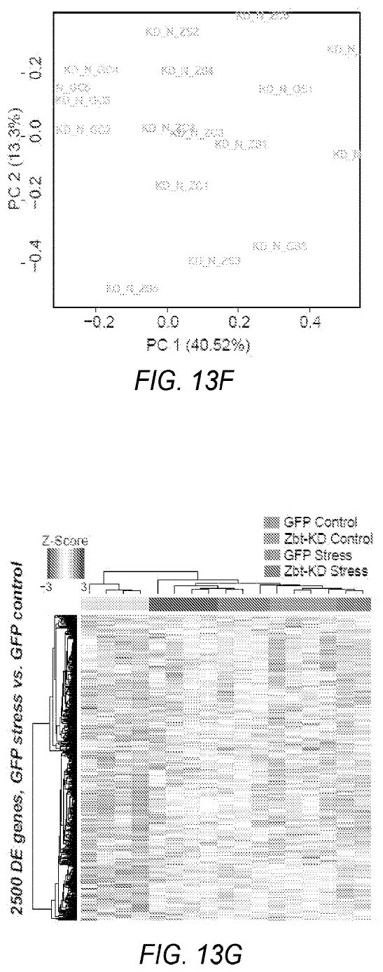

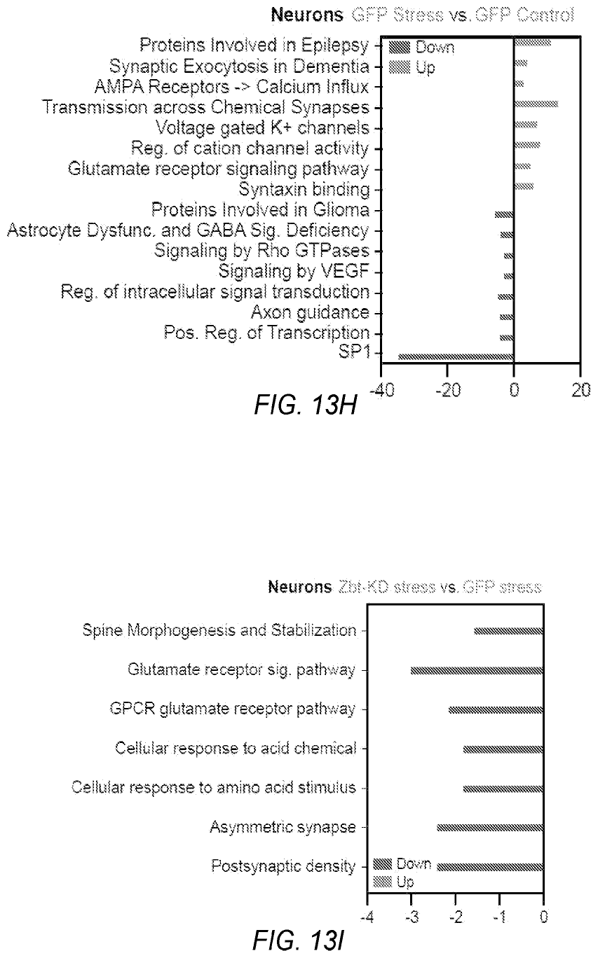

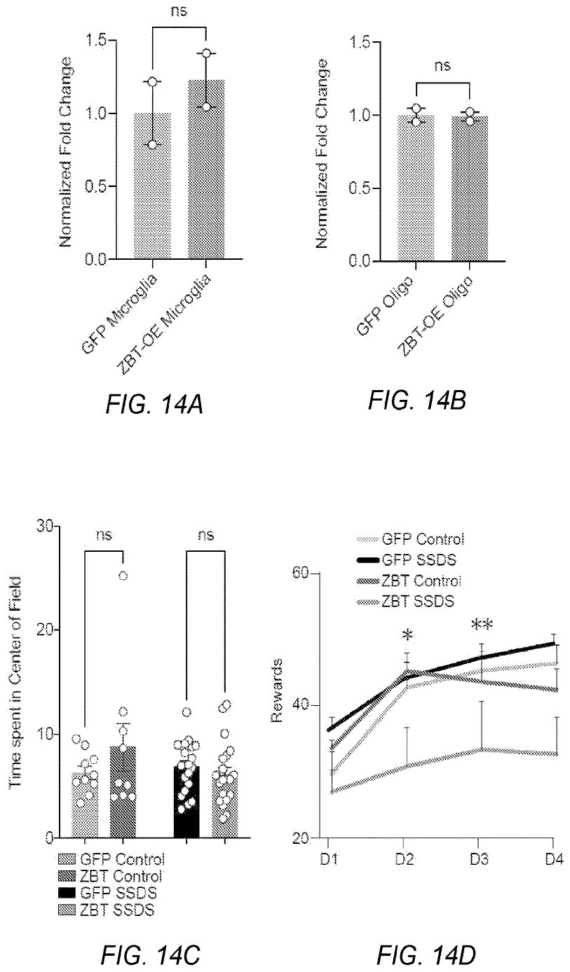

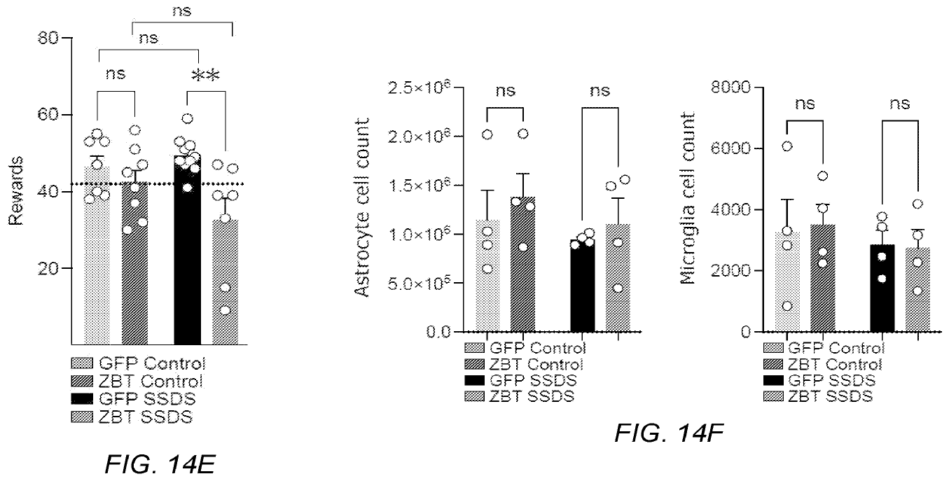

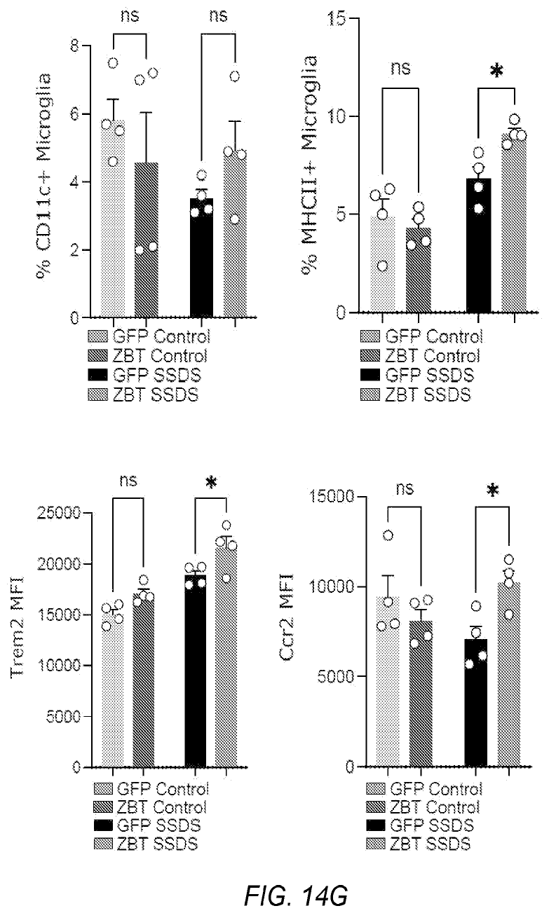

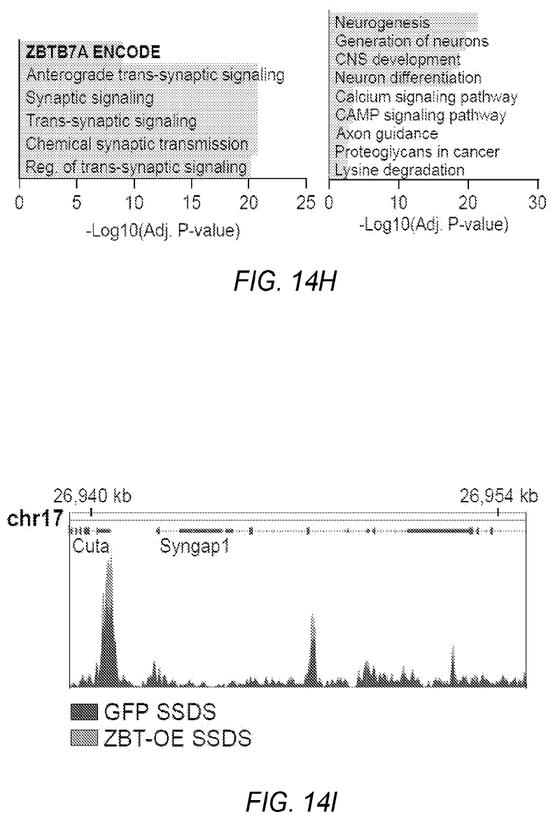

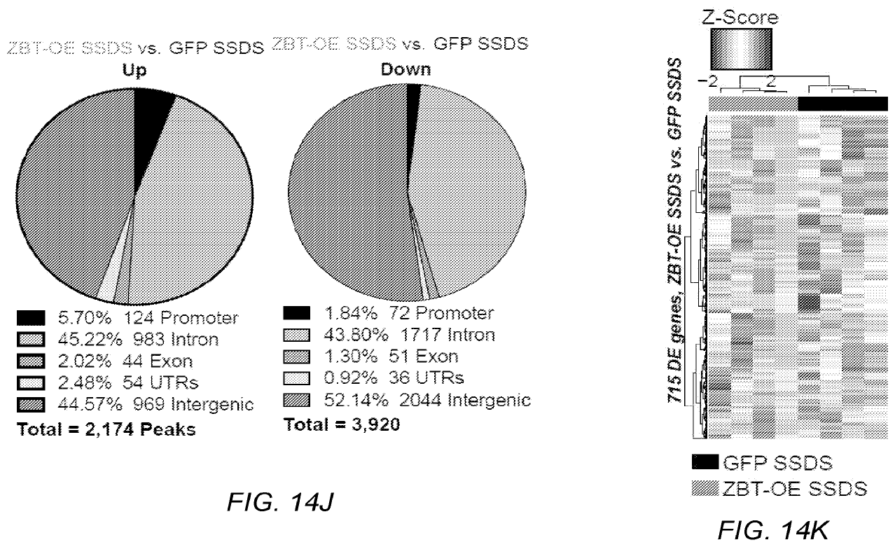

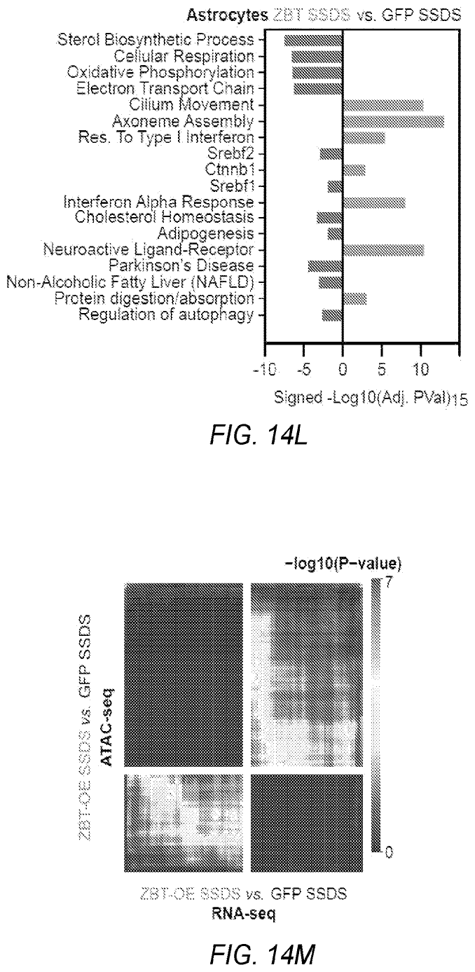

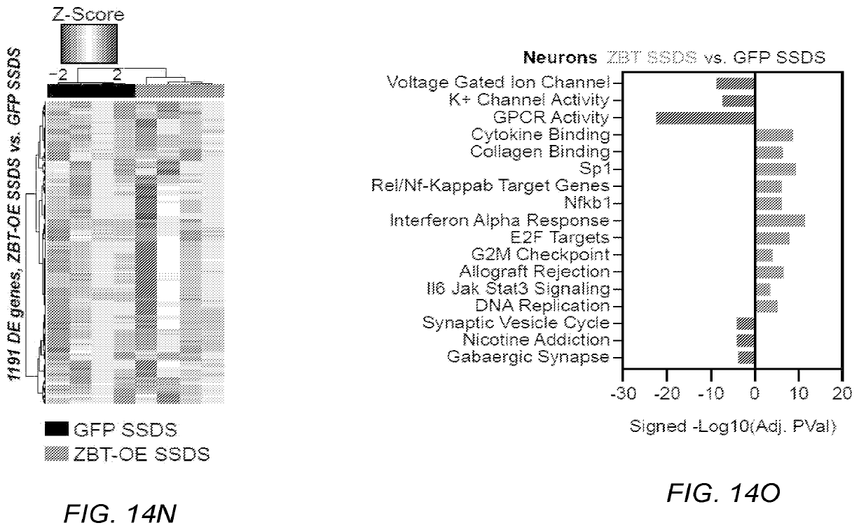

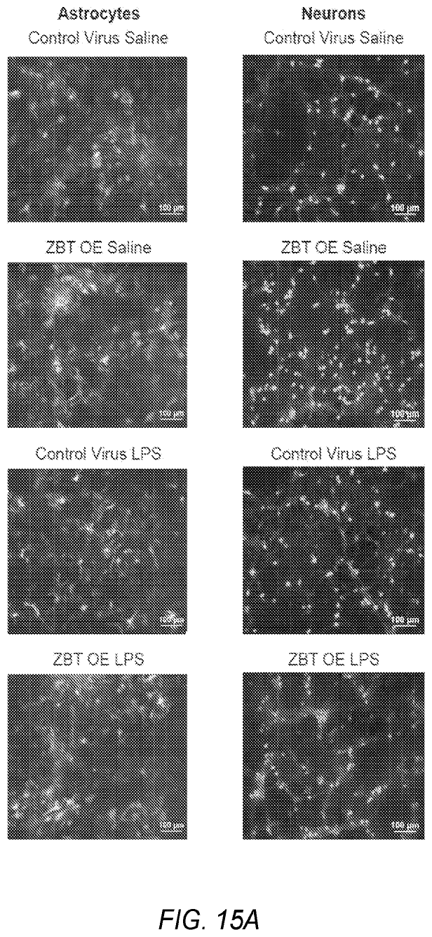

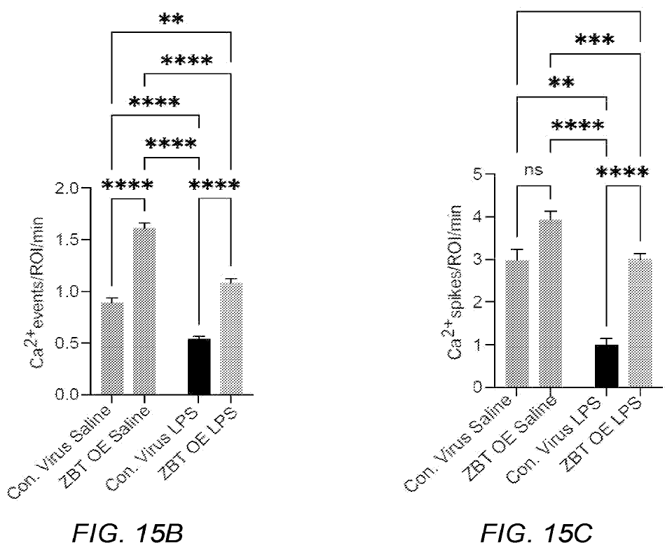

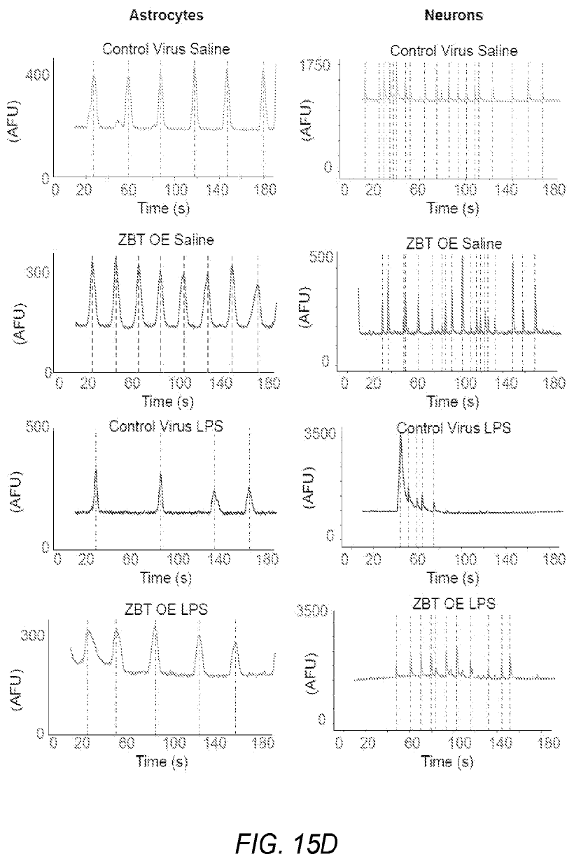

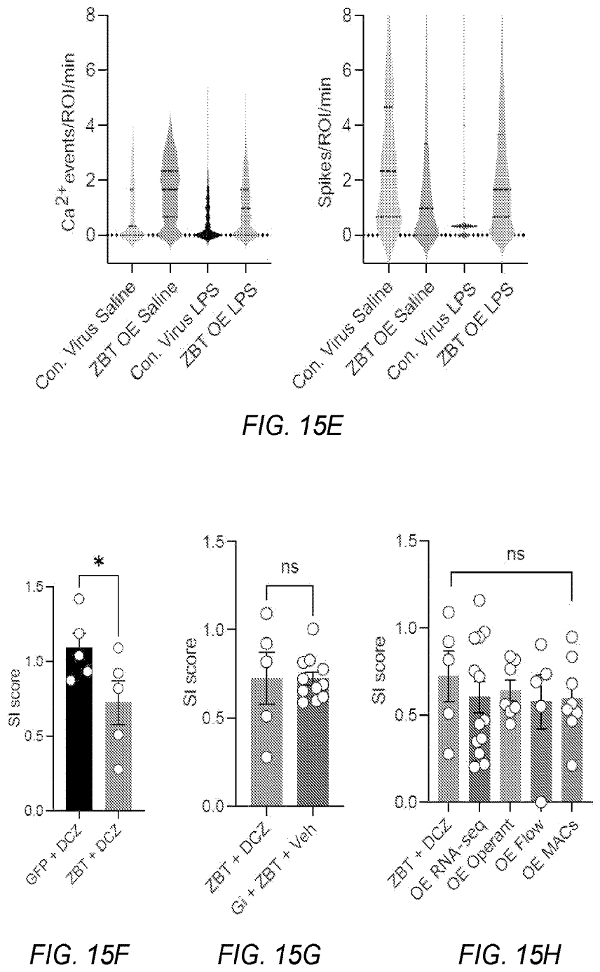

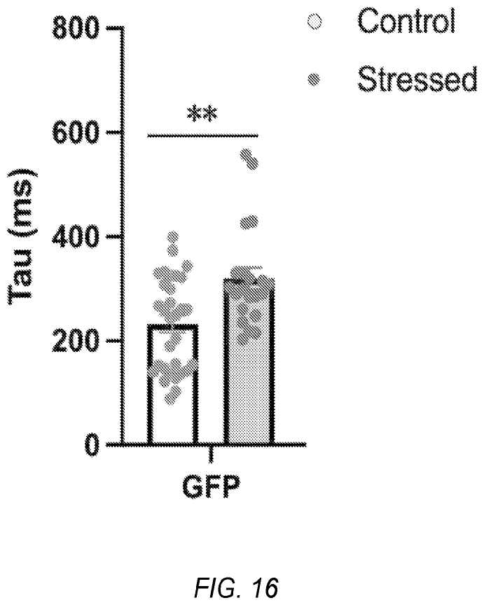

FIG. 2P is a plot of normalized fold change Zbtb7a mRNA expression in negative cell fraction post MACs-isolation of astrocytes and neurons, which is enriched for microglia, from chronically stressed OFC mouse tissues vs. control (n=4/group). Two-tailed Student's t-test [t6=1.053]. ns, p=0.332.

FIG. 3A is a schematic of experimental timeline with chronic social defeat stress (CSDS) paradigm performed after rAAV6 injection into OFC, followed by behavioral test and tissue collection for molecular analyses.

FIG. 3B is a plot of normalized fold change of qPCR Zbtb7a gene expression from OFC tissues transduced with Zbt-KD virus vs. miR-neg-GFP (GFP), with n=4/group. Two-tailed Student's t-test [t6=5.430]. ** p=0.0016.

FIG. 3C is an odds ratio analysis on differential OCR sets for indicated comparisons.

FIG. 3D is a Venn diagram of the number of shared and distinct OCRs between GFP control animals under high stress vs. Zbt-KD animals under low stress. Numbers indicate differentially accessible peaks, “J” indicates the Jaccard index.

FIG. 3E is a Venn diagram of the number of shared and distinct OCRs between GFP control animals under low stress vs. Zbt-KD animals under high stress. Numbers indicate differentially accessible peaks, “J” indicates the Jaccard index.

FIG. 3F is a plot of GO analysis for gene targets of overlapped (rescued) OCRs in Zbt-KD stress vs. GFP-stress.

FIG. 3G is a rank-rank hyper-geometric overlap (RRHO) comparing gene expression for the indicated comparisons. Each pixel represents the overlap between differential transcriptomes, with the significance of overlap of a hypergeometric test color-coded.

FIG. 3H is a rank-rank hyper-geometric overlap (RRHO) comparing gene expression for the indicated comparisons. Each pixel represents the overlap between differential transcriptomes, with the significance of overlap of a hypergeometric test color-coded.

FIG. 31 is a plot of clustering of Zbt-KD vs. GFP (+/−) chronic stress groups at 2513 DE genes (FDR<0.1) between ZBT-KD stress and GFP stress.

FIG. 3J is a gene-set enrichment analysis (GSEA) of RNA-seq profiles from indicated comparisons. Signed Log10 (Adj. P-value) was calculated based on direction of LogFC of each gene set.

FIG. 3K is a plot of GO analysis for 2,513 DE genes (FDR<0.1) between GFP stress and GFP control groups, separated by up/down regulation.

FIG. 3L is a plot of GO analysis for 119 DE genes between Zbt-KD stress and GFP stress groups, separated by up/down regulation.

FIG. 3M is a plot of Social interaction scores. 2-way ANOVA main effect of interaction [F1,49=13.97], p=0.0005. Tukey's MC test, GFP control vs. GFP stress **** p<0.0001. GFP Stress vs. Zbtb 7a KD stress **** p<0001. GFP control vs. Zbtb7a KD control ns, p=0.4909.

FIG. 3N is a plot of Pavlovian cue-reward association task. “D”=Day of task. Mixed Effects analysis, main effect of time×condition [F9,83=2.234]p=0.0275.

Dunnet's MC test, D2: control GFP vs. stress GFP *p=0.0399.

FIG. 30 is a plot of individual values for day two of the task shown in FIG. 3N.

FIG. 3P is a plot of effort-based operant reward learning task on FRI schedule, “D”=Day of task. Mixed Effects analysis main effect of virus×stress [F1,31=7.926]p=0.0084.

FIG. 3Q is a plot of individual values for day four of the task shown in FIG. 3P. 2-way ANOVA main effect of interaction [F1,30=5.411]p=0.0269. Sidak's MC test GFP control vs. Zbt-KD control, ns, p=0.7878. GFP stress vs. Zbt-KD stress *p=0.0146. All data graphed as means±SEM.

FIG. 4A is a plot of normalized fold change of qPCR Zbtb7a gene expression from OFC tissues transduced with ZBTB7A OE virus vs. GFP, n=5/group. Student's two-tailed t-test [t8=2.599]*p=0.0317.

FIG. 4B is a plot of normalized fold change of qPCR expression levels of Zbtb7a in MACs-isolated astrocytes, n=4/group. Student's two-tailed t-test [t6=3.458]*p=0.0135.

FIG. 4C is a plot of normalized fold change of qPCR expression levels of Zbtb7a in MACs-isolated neurons, n=4/group. Student's two-tailed t-test [t6=1.454]*p=0.1962.

FIG. 4D is a schematic of an experimental timeline with SSDS paradigm performed after rAAV6 injection into OFC, followed by behavioral tests and tissue collection for RNA-seq.

FIG. 4E is a plot of social interaction scores with a 2-way ANOVA main effect of stress [F1,52=8.144], p=0.0062, main effect of virus [F1,52=7.730], p=0.0075. Sidak's MC test, GFP control vs. GFP stress ns, p=0.2788. GFP stress vs. ZBT-OE stress ** p=0.0041. ZBT-OE control vs. ZBT-OE stress *p=0.0286. GFP control vs. ZBT-OE control n.s. p=0.4480

FIG. 4F is a plot of results of forced swim tests with a 2-way ANOVA main effect of interaction [F1,50=4.129], p=0.0475, main effect of stress [F1,50=4.993], p=0.0475. Sidak's MC test, GFP control vs. GFP stress ns, p=0.9876. GFP Stress vs. ZBT-OE stress ** p=0.0070.

FIG. 4G is a plot of percent sucrose consumption, with a 2-way ANOVA main effect of virus [F1,64=5.486], p=0.0223. Sidak's MC test, GFP control vs. GFP stress n.s., p=0.7882. GFP Stress vs. ZBT-OE stress *p=0.0166. ZBT-OE control vs. ZBT-OE stress n.s. p=0.1046. GFP control vs. ZBT-OE control n.s., p=0.6477.

FIG. 4H is a plot of percent correct trials in reversal learning paradigm. “B” indicates Baseline day, “R” indicates Reversal phase day. 3-way ANOVA, main effect of test day×stress [F9,261=4.529]p<0.0001.

FIG. 4I is a plot of individual values for day seven as shown in FIG. 4H. 2-way ANOVA, main effect of virus [F1,30=9.017], p=0.0054. Sidak's MC test, GFP control vs. GFP stress ns, p=0.9797. GFP Stress vs. ZBT-OE stress ** p=0.0013. ZBT-OE control vs. ZBT-OE stress *p=0.0389. GFP control vs. ZBT-OE control n.s., p=0.7280.

FIG. 4J is a RRHO comparing gene expression between indicated comparisons, in the context of mild stress.

FIG. 4K is a RRHO comparing gene expression between indicated comparisons, in the context of mild stress.

FIG. 4L is a plot of clustering of ZBT-OE vs. GFP (+/−) SSDS groups at 1,903 DE genes between ZBT-OE SSDS and GFP SSDS.

FIG. 4M is a GSEA of RNA-seq profiles from indicated comparisons.

FIG. 4N is a plot of GO pathway analysis of gene targets associated with differentially expressed [positive values are more accessible, negative values are less accessible]chromatin regions between ZBT-OE SSDS and GFP OE SSDS.

FIG. 40 is a Venn diagram [top] and odds ratio analysis [bottom] of the shared and distinct OCRs from ATAC-seq diffreps analysis between indicated conditions. All data graphed as means±SEM.

FIG. 4P is a Venn diagram [top] and odds ratio analysis [bottom] of the shared and distinct OCRs from ATAC-seq diffreps analysis between indicated conditions. All data graphed as means±SEM.

FIG. 5A is a schematic of an experimental timeline with subthreshold stress paradigm performed after AAV6 injection into OFC, followed by slice electrophysiology recordings.

FIG. 5B is a plot of an input-output (I-O) curve constructed by recording fEPSPs in response to stimuli ranging from 100-800 uA. 3-way ANOVA, main effect of stimulus intensity×virus×stress [F8,480=2.626]p=0.0080.

FIG. 5C is a plot of individual values for (I-O) curve, area under curve (A.U.C). 2-way ANOVA main effect of interaction [F1,59=4.062], p=0.0484. Sidak's MC test, GFP control vs. GFP stress ns, p=0.1923. GFP Stress vs. ZBT-OE stress *p=0.0295. ZBT-OE control vs. ZBT-OE stress n.s., p=0.4230. GFP control vs. ZBT-OE control n.s., p=0.9597.

FIG. 5D is a plot of a rundown stimulation from a single 30-s train delivered at 10 Hz. The percentage change in fEPSP amplitude from baseline was calculated during and post-10 Hz stimulation. 3-way ANOVA, main effect of stimulus×virus [F29,1334=3.376]p<0.0001, main effect of stress×virus [F1,46=4.356]p=0.0425.

FIG. 5E is a plot of individual values for delta fEPSP amplitude (% baseline) between end of 10 Hz stimulation and Is after end of stimulation train. 2-way ANOVA main effect of virus [F1,46=6.115], p=0.0172, main effect of stress [F1,46=8.454], p=0.0056. Sidak's MC test, GFP control vs. GFP stress ns, p=0.5172. GFP Stress vs. ZBT-OE stress *p=0.0207. ZBT-OE control vs. ZBT-OE stress ** p=0.0059. GFP control vs. ZBT-OE control n.s., p=0.5172.

FIG. 5F is a series of fluorescent microscopy images from immunohistochemistry (IHC) validation of hsyn-hM4D (Gi)-mCherry (in red) and GFAP-ZBT OE (in green) localized in astrocytes (GFAP, in yellow) and DAPI (in blue). Images taken at 10× magnification.

FIG. 5G is an experimental schematic of chemogenetics experiment, in which SSDS is performed on a cohort of mice expressing hM4D (Gi)-mCherry (+/−) ZBT OE, (+/−) DCZ.

FIG. 5H is a plot of social interaction scores, with a 2-way ANOVA main effect of virus [F1,41=10.11], p=0.0028, main effect of agonist [F1,41=10.65], p=0.0022. Sidak's MC test, Gi+GFP stress+vehicle vs. Gi+GFP stress+DCZ ns, p=0.3880. Gi+ZBT-OE stress+vehicle vs. Gi+ZBT-OE stress+DCZ *p=0.0040. All data graphed as means±SEM.

FIG. 6A is a plot of principal component analysis of sample gene expression levels.

FIG. 6B is a density plot of transformed data.

FIG. 6C is a plot of the distribution of transformed data.

FIG. 6D is an nalysis of scale-free fit index for possible soft-thresholding powers (B).

FIG. 6E is an analysis of mean connectivity for possible soft-thresholding powers.

FIG. 6F is a plot of GO analysis for 1,450 DE genes between MDD and control groups, separated by up/down regulation.

FIG. 7A is a plot of the fraction of uniquely mapped, non-duplicated, non-chrM paired-end reads compared to all reads in raw sequencing files.

FIG. 7B is a plot of the number of uniquely mapped, non-duplicated, non-chrM paired-end reads.

FIG. 7C is a plot of the fraction of duplicated to uniquely mapped paired-end reads.

FIG. 7D is a plot of the fraction of mitochondrial DNA reads to uniquely mapped, non-duplicated paired-end reads.

FIG. 7E is a plot of the number of OCRs (called per sample).

FIG. 7F is a plot of the fraction of reads in OCRs (FRiP).

FIG. 7G is a plot of GC-content in a consensus set of OCRs.

FIG. 7H is a plot of median insert size of OCRs.

FIG. 7I is a genotype check based on pair-wise comparison of genotypes called from ATAC-seq samples. Pairs of neuronal and non-neuronal samples supposedly originating from the same person have distinctly higher scores (at right) than pairs of samples from different individuals (at left).

FIG. 8A is a diagram of the first two phases of a workflow of the analytical steps for ATAC-seq samples split into three phases: (1) per sample processing and (2) joint processing for quality control. Workflow for analyses of samples meeting the quality control criteria is shown in FIG. 8B.

FIG. 8B is a diagram of the first two phases of a workflow for analyses of samples meeting the quality control criteria. The first two phases of the workflow are shown in FIG. 8A.

FIG. 9A is a plot showing a summary of P-value ranking for the reported set of 203 differentially accessible OCRs within differentially analyses results generated on the datasets of non-neuronal samples with randomly permuted MDD and Control status (n=100 permuted datasets).

FIG. 9B is a plot of per-OCR distribution of P-value ranking for the reported set of 203 differentially accessible OCRs within differentially analyses results generated on the datasets of non-neuronal samples with randomly permuted MDD and Control status (n=100 permuted datasets).

FIG. 9C is a series of plots showing the performance of machine learning classifiers built on the reported set of 203 differential OCRs and 203 random OCRs. To enable the robust performance evaluation, the repeated 5-fold cross-validation was applied (krepeat=10); additionally, the whole process was repeated 10 times with different sets of 203 randomly selected OCRs.

FIG. 10A is a plot of GO analysis with CellMarker Augmented Database for genes in non-neuronal specific promoters, filtered by logFC>1, (+/−) 3000 bp from TSS.

FIG. 10B is a plot of correlation between t-statistics of MDD-associated changes in the epigenome (ATAC-seq; OCR needs to overlap gene's TSS) and MDD-associated changes in transcriptome (RNA-seq). The P-value threshold of filtering ATAC-seq OCRs is shown on the x-axis (P-value calculated by two-sided t-test), and the Pearson correlation coefficient is shown on the y-axis. The size of the point denotes the log 10 count of promoter-gene combinations.

FIG. 10C is a series of plots showing quantification of MDD-specific ATAC-seq gene target mRNA in FAN-sorted non-neuronal nuclei from human MDD vs. control in OFC. Bar graphs display normalized fold change of target genes associated with MDD-specific OCRs relative to housekeeping gene (GAPDH) for control vs. MDD samples.

FIG. 10D is a heatmap of GSEA using general gene sets. Note that these changes were not restricted to pathways that are exclusive to non-neuronal cell-types. P-values for the top ten enriched gene sets in each peakset. “#” Significance at FDR<5%.” · “: Nominal significance. “NA”: not available. “Bi”: Biocarta. “GO”: Gene Ontology. “KG”: Kegg. “Re”: Reactome. Student's two-tailed t-tests were performed for statistical comparisons, *=p<. 05, **=p<. 01. Data displayed as mean (+/−SEM).

FIG. 11A is a plot of consensus score from the Human Protein Atlas for expression in human brain for each factor. The mRNA expression data is derived from deep sequencing of RNA (RNA-seq) from 37 different normal tissue types.

FIG. 11B is a plot of normalized fold change for mRNA expression for ELF1 in bulk human OFC tissues, control vs. MDD.

FIG. 11C is a pair of bar graphs for number of bound ZBTB7A TFBS detected exclusively in MDD case or control samples from neuronal and non-neuronal cells (left) and exclusively in non-neuronal and neuronal populations (mixed MDD/control).

FIG. 11D is a plot of overlap between DE genes from Female MDD vs. control OFC tissues and ENCODE consensus target gene sets via EnrichR, plotted by rank (y-axis) and -log 10 (adjusted p-value) on the x-axis and by fill color. Bubble size displays the number of overlapping genes for each term.

FIG. 11E is a plot of overlap between ZBTB7A target genes (from TRANSFAC) and ARCHS4 human tissue expression reference gene sets via EnrichR. plotted by rank (y-axis) and -log 10 (Adjusted P-value) on the x-axis and by fill color. Bubble size displays the number of overlapping genes for each term.

FIG. 11F is a plot of expression of ZBTB7A in human primary cultured astrocytes treated with ZBTB7A OE lentivirus vs. RFP empty vector control virus.

FIG. 11G is a series of bar graphs showing normalized fold change of mRNA expression in ZBT-OE vs. RFP human primary cultured astrocytes for the listed gene targets.

FIG. 11H is a plot of normalized fold change of cell-type specific marker genes in human primary cultured astrocytes.

FIG. 11I is a plot of normalized fold change of ZBTB7A mRNA expression in cultured human astrocytes treated with saline vs. LPS.

FIG. 11J is a plot of normalized fold change of Zbtb 7a mRNA expression in cultured mouse astrocytes treated with saline vs. LPS.

FIG. 11K is a plot of the social interaction ratio for control vs. chronically stressed CSDS mouse groups at 48 h post-stress and 21 d post-stress.

FIG. 11L is a plot of the normalized fold change protein expression of Zbtb7a in mouse OFC bulk tissues collected from control vs. chronically stressed mouse groups at 21 d post-stress.

FIG. 11M is a plot of qPCR expression data for astrocyte-specific gene Aldhlal in MACs-isolated cell fractions.

FIG. 11N is a plot of qPCR expression data for neuron-specific Rbfox3 (NeuN in MACs-isolated cell fractions).

FIG. 110 is a plot of qPCR expression data for cell type-specific genes in negative fraction from MACs-isolated astrocyte and neuron cell fractions, showing the negative fraction is enriched for microglia marker Cd11b. Student's two-tailed t-tests or 1-way ANOVA with MC tests were performed for statistical comparisons.

FIG. 11P is a plot of qPCR expression data for Zbtb7a in MACs-isolated astrocyte vs. neuron cell fractions. Data presented as mean (+/−SEM). *=p<0.05, **=p<0.01, ***=p<0.001, ****=p<0.0001.

FIG. 11Q is a series of 20× IHC images showing Zbtb7a protein is expressed in mouse OFC astrocytes, depicts overlap of Zbtb7a with astrocyte-specific marker Gfap.

FIG. 11R is a plot of Thresholded Mander's coefficient describing overlap of color channels of interest for the images of FIG. 11Q.

FIG. 12A is a plot of qPCR expression data for Astrocyte-specific Aldhlal in MACs-isolated cell fractions.

FIG. 12B is a plot of qPCR expression data for Neuron-specific Rbfox3 (NeuN) in MACs-isolated cell fractions.

FIG. 12C is a plot of qPCR expression data for Microglia-specific Iba in MACs-isolated cell fractions.

FIG. 12D is a plot of qPCR expression data for Immature Oligodendrocyte-specific Pdgfra in MACs-isolated cell fractions.

FIG. 12E is a plot of qPCR expression levels of the GFP transgene in MACs-isolated neurons vs. astrocytes from AAV6-GFAP-miR-neg-GFP virally-transduced OFC mouse tissues.

FIG. 12F is a series of representative IHC images of OFC tissues transduced with an rAAV6 virus expressing ZBTB7A-GFP (in magenta) overlaid with a nuclear co-stain (DAPI in blue) and GFAP (in yellow) to show astrocyte-specific expression.

FIG. 12G is a plot of cell counts in OFC tissues transduced with AAV6-ZBTB7A-GFP of cells co-expressing Gfap/Zbtb7a or NeuN/Zbtb7a.

FIG. 12H is a pair of pie charts showing ATAC-seq diffReps analysis of differential accessibility between indicated conditions. The pie charts indicate distribution of differential accessibility events, stratified by genomic context for the indicated conditions and separated for up/down events.

FIG. 121 is a pair of pie charts showing ATAC-seq diffReps analysis of differential accessibility between indicated conditions. The pie charts indicate distribution of differential accessibility events, stratified by genomic context for the indicated conditions and separated for up/down events

FIG. 12J is a pair of pie charts showing ATAC-seq diffReps analysis of differential accessibility between indicated conditions. The pie charts indicate distribution of differential accessibility events, stratified by genomic context for the indicated conditions and separated for up/down events.

FIG. 12K is a chart showing Gene ontology (GO) pathway analysis of differentially accessible promoters from Zbt-KD vs. GFP stress [less accessible promoters, top] and GFP Stress vs. GFP Control [more accessible promoters, bottom].

FIG. 12L is a plot showing principal component analysis of sample gene expression levels in astrocyte-specific RNA-seq.

FIG. 12M is a RRHO comparing gene expression and chromatin accessibility for the indicated comparisons.

FIG. 12N is a RRHO comparing gene expression and chromatin accessibility for the indicated comparisons.

FIG. 120 is a Venn-diagram overlap of differentially expressed (DE) genes in MACs-isolated astrocytes comparing Zbt-KD stress vs. GFP stress, with GFP stress vs. GFP control.

FIG. 12P is a RRHO comparing gene expression for the indicated comparisons, in bulk OFC tissue.

FIG. 12Q is a RRHO comparing gene expression for the indicated comparisons, in bulk OFC tissue.

FIG. 12R is a heatmap showing GSEA for ZBT stress vs. GFP Stress and GFP Stress vs. GFP Control stress comparisons in bulk OFC tissue. Top enriched gene sets are shown, heatmap depicts signed-log 10 (Adj. P-val) for gene set enrichment based on logFC direction.

FIG. 12S is a plot of clustering of groups at 1,711 DE genes between GFP stress and GFP control in bulk OFC.

FIG. 12T is a Venn-diagram overlap of differentially expressed (DE) genes in bulk OFC tissues comparing Zbt-KD stress vs. GFP stress, with GFP stress vs. GFP control.

FIG. 12U is a chart showing GO pathway analysis of 356 rescued DE genes between Zbt-KD Stress and GFP Stress in bulk OFC tissues. Data were analyzed with Student's two-tailed t-tests. *=p<0.05, **=p<0.01, ***=p<0.001, ****=p<0.0001. All data graphed as means±SEM.

FIG. 13A is a heatmap showing unsupervised clustering of normalized read count values in MACs-isolated astrocytes and neurons for 239 astrocyte-enriched genes.

FIG. 13B is a heatmap showing unsupervised clustering of normalized read count values in MACs-isolated astrocytes and neurons for 279 neuron enriched genes

FIG. 13C is a RRHO comparing gene expression for the indicated comparisons, in MACS-isolated neurons.

FIG. 13D is a RRHO comparing gene expression for the indicated comparisons, in MACS-isolated neurons.

FIG. 13E is a heatmap of GSEA for ZBT stress vs. GFP Stress and GFP Stress vs. GFP Control stress comparisons in MACs-isolated neurons. Top enriched gene sets are shown, heatmap depicts signed-log 10 (Adj. p-val) for gene set enrichment based on logFC direction.

FIG. 13F is a plot showing principal component analysis of sample gene expression levels in neuron-specific RNA-seq.

FIG. 13G is a plot showing clustering of groups at 2,701 DE genes between GFP-stress and GFP control in bulk OFC.

FIG. 13H is a chart showing GO analysis for 2,701 DE genes between GFP stress and GFP control groups, separated by up/down regulation.

FIG. 13I is a chart showing GO analysis for 92 DE genes between Zbt-KD stress and GFP stress groups, separated by up/down regulation.

FIG. 14A is a plot of qPCR expression levels of Zbtb7a in MACs-isolated microglia from AAV6-GFAP-ZBT OE transduced virally-transduced OFC mouse tissues.

FIG. 14B is a plot of qPCR expression levels of Zbtb7a in MACs-isolated oligodendrocytes from AAV6-GFAP-ZBT OE transduced virally-transduced OFC mouse tissues.

FIG. 14C is a plot of time spent(s) in the center of the field during open field test.

FIG. 14D is a plot of results of the Pavlovian cue-reward association task. “D” indicates Day of test.

FIG. 14E is a plot of Individual values for day 2 of task shown in FIG. 14D.

FIG. 14F is a pair of plots of number of astrocytes [left], and microglia [right]per organ.

FIG. 14G is a series of plots of percent CD11c+microglia [far left], percent MHCII+microglia [left], Trem2 MFI [right] and Ccr2 [far right]MFI100 in virally transduced ZBT OE vs. GFP mice (+/−SSDS) OFC via flow cytometry.

FIG. 14H is a chart of GO pathway analysis of gene targets associated with downregulated [left, in blue] and upregulated [right, in red]DE genes between ZBT OE SSDS vs. GFP SSDS mice in bulk OFC tissues.

FIG. 14I is a plot of representative pile-up traces of cell specific ATAC-seq signal overlapping Syngap1 gene.

FIG. 14J is a pair of pie charts showing ATAC-seq diffReps analysis of differential accessibility comparing ZBT-OE SSDS vs. GFP SSDS. Pie charts indicate distribution of differential accessibility events, stratified by genomic context.

FIG. 14K is a heatmap showing Clustering of ZBT-OE vs. GFP (+/−) SSDS groups (n=4/group) at DE genes between ZBT-OE SSDS and GFP SSDS astrocytes (n=715 DE genes).

FIG. 14L is a chart showing GO analysis for DE genes (FDR<0.1) between ZBT-OE SSDS and GFP SSDS groups in MACs-isolated astrocytes (n=715 DE genes).

FIG. 14M is a heatmap showing a RRHO comparing gene expression profile of MACs-isolated astrocytes with MACS-isolated astrocyte chromatin accessibility for indicated conditions.

FIG. 14N is a heatmap showing clustering of ZBT-OE vs. GFP (+/−) SSDS groups at DE genes between ZBT-OE SSDS and GFP SSDS in MACs-isolated neurons (n=1,191 DE genes).

FIG. 140 is a chart showing GO analysis for DE genes (FDR<0.1) between ZBT-OE SSDS and GFP SSDS groups in MACs-isolated neuron (n=1,191 DE genes) samples, separated by up/down regulation. Data were analyzed with Student's two-tailed t-tests or with 2-way ANOVA, or 3-way ANOVA, followed by 2-Way ANOVAs for MC comparisons, *=p<0.05, **=p<0.01. All data graphed as means±SEM.

FIG. 15A is a series of representative images showing gCAMP6f-expressing cells in either astrocyte-treated or neuron-treated primary co-cultures.

FIG. 15B is a plot of mean frequency of Ca2+events detected in astrocytes expressing gCAMPf. “Con.” Indicates Control.

FIG. 15C is a plot of mean frequency of Ca2+event detected in neurons expressing gCAMP6f. “Con.” Indicates Control.

FIG. 15D is a series of representative traces for calcium event frequencies in astrocytes [left] and neurons [right].

FIG. 15E is a pair of violin plots depicting individual values for (right) astrocyte [n-623 cells control virus saline, n=559 cells ZBT OE saline, n=747 cells control virus LPS, and n=517 cells ZBT OE LPS] and (left)neuronal [n=135 cells control virus saline, n=1277 cells ZBT OE saline, n=238 cells control virus LPS, and n=1324 cells ZBT OE LPS] calcium events.

FIG. 15F is a plot of social interaction scores for ZBT OE SSDS vs. GFP SSDS mice injected with DCZ.

FIG. 15G is a plot of social interaction scores for ZBT OE SSDS vs. ZBT OE+Gi Dreadd+vehicle

FIG. 15H is a plot of comparison of SI score across multiple cohorts of ZBT OE SSDS animals. Data were analyzed with Student's two-tailed t-tests or with 1-way ANOVA plus Tukey's MC test, *=p<0.05, **=p<0.01, ***=p<0.001, ****=p<0.0001. All data graphed as means±SEM.

FIG. 16 is a plot of synaptic glutamate clearance (Tau; ms) measured by whole-cell patch-clamp in orbitofrontal cortex brain slices from mice transduced with GFP (control vector) in astrocytes under stressed and control conditions, showing a significantly increased Tau (ms)—i.e., decreased glutamate clearance—for the stressed condition.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For purposes of the present disclosure, the following terms are defined.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “the agent” includes reference to one or more agents known to those skilled in the art, and so forth.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 10% or within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to provide written description support for a claim limitation of, e.g., “0.98X.” The terms “about” and “approximately,” particularly in reference to a given quantity, encompass and describe the given quantity itself. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 5, 10, or 15 mg” is equivalent to “about 5, about 10, or about 15 mg.” An “equivalent dose” refers to an equivalent dose of an active agent based on bioavailability. An equivalent dose based on bioavailability can be determined by comparing the extent and rate of drug absorption of two or more dosages (e.g., dosages formulated as an intranasal formulation and as an intravenous formulation, respectively) of an active agent, for example, by determining the area under the blood or plasma concentration-time curve (AUC) and/or the maximum concentration (Cmax), respectively. Accordingly, as used herein, an equivalent dose based on bioavailability is present when the two dosages each exhibit an AUC and/or Cmax within about 80% to about 125% of one another.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease. In some embodiments, “treatment” includes resolution of a particular disorder, including a reduction in one or more symptoms of the disorder and/or a reduction in in the severity of one or more symptoms associated with the disorder.

“Administering” or “administration” refer to the physical introduction of a therapeutic agent to a subject, using any of the various methods and delivery systems known to those skilled in the art. Routes of administration can include oral, intravenous, intranasal, intramuscular, subcutaneous, or other parenteral routes of administration, for example by injection or infusion (e.g., intravenous infusion). Administration can also be performed, for example, once, a plurality of times, and/or over one or more extended periods.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human.

An “effective amount” or “therapeutically effective amount” of a therapeutic agent is any amount of the drug that, when used alone or in combination with one or more additional therapies, slowing down the onset of a psychiatric disorder or promotes regression of the disorder evidenced by a decrease in severity of disorder symptoms, an increase in frequency and duration of disorder symptom-free periods, or ameliorating an impairment or disability due to the disorder affliction. The ability of one or more additional therapies to promote disorder regression can be evaluated using a variety of methods known to the skilled practitioner, such as in human subjects during clinical trials, in animal model systems predictive of efficacy in humans, or by assaying the activity of the agent in in vitro assays.

As used herein, a measure of a treatment effect is “clinically meaningful” based on the practical importance of a treatment effect. For example, whether the treatment effect has a real genuine, palpable, and/or noticeable effect on the subject (e.g., a lack of clinically meaningful effect occurs when the difference in the subject is small enough that it may be considered similar, such as prior to and after administration of a treatment as provided herein). One skilled in the art would recognize whether a particular effect is “clinically meaningful.” For example, a subject having a baseline score indicating severe depression and/or suicidality (using any of the scales described herein) and a post-treatment score indicating remission of the severe depression and/or suicidality would be a clinically meaningful effect.

A subject that is “not responsive” refers to a subject that has been, or is currently being, treated with one or more therapies that are not providing a clinically meaningful change towards the desired outcome (e.g., subjects that are not responsive includes patients that are refractory to a particular treatment). For example, a subject may exhibit no measurable change in response to therapy. A non-responsive subject could also, for example, exhibit a positive change in a depression scale score, but the change is not clinically meaningful.

As used herein, a psychiatric evaluation or side effect profile test score that is “substantially similar” or “substantially the same” as a reference score, corresponds to the same score, with a skilled artisan understanding that particular test scores may vary to a reasonable extent (such as +10%) while still describing a given value, due to, for example, experimental error, routine subject-to-subject evaluation, and routine statistical analysis.

The phrase “pharmaceutically acceptable” indicates that the substance or composition must be compatible chemically and/or toxicologically, with the other ingredients comprising a formulation, and/or the mammal being treated therewith. The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

As used in the methods described herein, the term “reducing” refers to a reduction in the indicated parameter relative to the baseline measurement (or measurements) of the same parameter in the subject taken prior to the initiation of administration with the pharmaceutical compositions described herein, or a reduction in the indicated parameter relative to the baseline measurement (or measurements) of the same parameter. In some embodiments, the same parameter is measured in a healthy subject (for example, a subject that does not have a neurological disorder as described herein). In some embodiments, the same parameter is measured relative to another treatment modality (for example, the standard of care treatment for a neurological disorder as described herein). Similarly, the term “increasing,” as used herein, refers to an increase in the indicated parameter relative to the baseline measurement (or measurements) of the same parameter in the subject taken prior to the initiation of administration with the pharmaceutical compositions described herein, or an increase in the indicated parameter relative to the baseline measurement (or measurements) of the same parameter. In some embodiments, the same parameter is measured in a healthy subject (for example, a subject that does not have a neurological disorder as described herein). In some embodiments, the same parameter is measured relative to another treatment modality (for example, the standard of care treatment for a neurological disorder as described herein).

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to subjects in need of treatment. However, the observation of synergy in in vitro models or in vivo models can be predictive of the effect in humans and other species and in vitro models or in vivo models exist, as described herein, to measure a synergistic effect. Exemplary synergistic effects includes, but are not limited to, enhanced therapeutic efficacy, decreased dose at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g., side effects and adverse events) of at least one of the therapeutic agents.

For example, a synergistic ratio of two therapeutic agents can be identified by determining a synergistic effect in, for example, an art-accepted in vivo model (e.g., an animal model) of depression (e.g., despair-based, reward-based, or anxiety-based mouse models).

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail herein.

### Zinc Finger and BTB Domain-Containing Protein 7A (ZBTB7A)

Zinc finger and BTB domain-containing protein 7A (ZBTB7A) is a chromatin remodeling protein that coordinates wide-ranging cellular activation programs, including NF-kB inflammatory transcription. ZBTB7A in humans is encoded by the ZBTB7A gene located at chromosome 19p13.3 (chr19: 4,043,303-4,066,899, GRCh38/hg38). An exemplary ZBTB7A mRNA polynucleotide sequence encoding ZBTB7A (NCBI Reference Sequence: NM_015898.4) is listed below as SEQ ID NO: 1:

GGAGAAACCAGGAAGGGAGTCGCGAGCATCATACGGGGCTTTGGCTGTAC
TGTACAGTTACTGTAGGGGCAGTGACGCCGCCGCCGCCGCGCGAGGGAGC
GACCAGGGCAGCGCGACGCGGGCAGAGCGAGCGCCGCGGACCCCGGAGGC
GAGCGGCCGAGGGAGCCCAGTCCCAACTTCGGGCCCCGGCCCCGCGCGCC
CCGGCCCGGCCCCGCGAGGTCTCGGCGCGGAAGATGGCCGGCGGCGTGGA
CGGCCCCATCGGGATCCCGTTCCCCGACCACAGCAGCGACATCCTGAGTG
GGCTGAACGAGCAGCGGACGCAGGGCCTGCTGTGCGACGTGGTGATCCTG
GTGGAGGGCCGCGAGTTCCCCACGCACCGCTCGGTGCTGGCCGCCTGCAG
CCAGTACTTCAAGAAGCTGTTCACGTCGGGCGCCGTGGTGGACCAGCAGA
ACGTGTACGAGATCGACTTCGTCAGCGCCGAGGCGCTCACCGCGCTCATG
GACTTCGCCTACACGGCCACGCTCACCGTCAGCACAGCCAACGTGGGTGA
CATCCTCAGCGCCGCCCGCCTGCTGGAGATCCCCGCCGTGAGCCACGTGT
GCGCCGACCTCCTGGACCGGCAGATCCTGGCGGCCGACGCGGGCGCCGAC
GCCGGGCAGCTGGACCTTGTAGATCAAATTGATCAGCGCAACCTCCTCCG
CGCCAAGGAGTACCTCGAGTTCTTCCAGAGCAACCCCATGAACAGCCTGC
CCCCCGCGGCCGCCGCCGCCGCTGCCAGCTTCCCGTGGTCCGCCTTTGGG
GCGTCCGATGATGACCTGGATGCCACCAAGGAGGCCGTGGCCGCCGCTGT
GGCCGCCGTGGCCGCGGGCGACTGCAACGGCTTAGACTTCTATGGGCCGG
GCCCCCCGGCCGAGCGGCCCCCGACGGGGGACGGGGACGAGGGCGACAGC
AACCCGGGTCTGTGGCCAGAGCGGGATGAGGACGCCCCCACCGGGGGTCT
CTTTCCGCCGCCGGTGGCCCCGCCGGCCGCCACGCAGAACGGCCACTACG
GCCGCGGCGGAGAGGAGGAGGCCGCCTCGCTGTCGGAGGCGGCCCCCGAG
CCGGGCGACTCTCCGGGCTTCCTGTCGGGAGCGGCCGAGGGCGAGGACGG
GGACGGGCCCGACGTGGACGGGCTGGCGGCCAGCACGCTGCTGCAGCAGA
TGATGTCATCGGTGGGCCGGGCGGGGGCCGCGGCGGGGGACAGCGACGAG
GAGTCGCGGGCCGACGACAAGGGCGTCATGGACTACTACCTGAAGTACTT
CAGCGGCGCCCACGACGGCGACGTCTACCCGGCCTGGTCGCAGAAGGTGG
AGAAGAAGATCCGAGCCAAGGCCTTCCAGAAGTGCCCCATCTGCGAGAAG
GTCATCCAGGGCGCCGGCAAGCTGCCGCGACACATCCGCACCCACACGGG
CGAGAAGCCCTACGAGTGCAACATCTGCAAGGTCCGCTTCACCAGGCAGG
ACAAGCTGAAGGTGCACATGCGGAAGCACACGGGCGAGAAGCCGTACCTG
TGCCAGCAGTGCGGCGCCGCCTTTGCCCACAACTACGACCTGAAGAACCA
CATGCGCGTGCACACGGGCCTGCGCCCCTACCAGTGCGACAGCTGCTGCA
AGACCTTCGTCCGCTCCGACCACCTGCACAGACACCTCAAGAAAGACGGC
TGCAACGGCGTCCCCTCGCGCCGCGGCCGCAAGCCCCGCGTCCGGGGCGG
GGCGCCCGACCCCAGCCCGGGGGCCACCGCGACCCCCGGCGCCCCCGCCC
AGCCCAGCTCCCCCGACGCCCGGCGCAACGGCCAGGAGAAGCACTTTAAG
GACGAGGACGAGGACGAGGACGTGGCCAGCCCCGACGGCTTGGGCCGGTT
GAATGTAGCGGGCGCCGGTGGAGGAGGTGACAGCGGAGGTGGCCCCGGGG
CCGCCACCGACGGTAACTTCACAGCCGGACTCGCCTAAAAACCAAAAAGA
GAAAACAGAAACCCGAGAAAGAGAGAGACAGAGAGAGAGAAAAAAAATCA
CCCACCACCCCCCCAAAAACACAAAAAAAGAAAATCTATCTATATACAGA
TATCTATATCTATATATATATATACAGATATATATATATGACGCGTCACA
GAATCTAGGGTAGCGCTTTCTCAGATTTCCCTCCTTTCTGACGTTTTTCT
CCCTCCGCAGGGGCCCCGGCCCTCCCTGGCTCCCCTTCCCCCCACCACCC
CATCGCTGGGTTTCGGGGCTTGGTTTGGGGTTTTTTGTAGGACACAAGGA
ATCCGAGACCCCGCACAGCCCCCTGGGCACCCGGCATGGGGCCTGGGGCC
CGATCCGAGGCCCTGGGCTGGGGGGAGGGTAGACGTGGGGGCGCTGGGGG
GGGACTGGGGTGGGCTTTTAATTTCCTCCCCTCGCTGGTTTCTATGAGTC
TTTCAGACAAGACCTTAAATGATTTCTGTCTGCTCTGAGCGGACGTTAAA
ATGGGCCCCCGTCCCCCGACCCGCACCCTCCTTCCTCAGGGCACTTACTA
AGGGAGGGGTCTCCCTCTCCATCTCCCCAGTGGCCTCCCCGCCTCCAACC
CTGCCTGCGGCCTCCCCCCGTCGCCCACCCCACGTCTCCTGGCCACTGAG
ACACAAACCTATTTATTTCTAGGCCTGGAGAAAGGAGATCGGACTGGGGT
TCCCGGTGGGGCGCCAGGATGGCTCCTGGGGGTGCTCCTGCCGCCTTCCT
TCACGGCACTTACAACCGGCGGGACCCCCAGGGACCACCCCTCAGGGCGC
CCCCCCACCCCCGCCCGGTCCACCTAGACCCCCACGTTTGGAGATTCAAA
ACTTCTGTCTTCGTCCTCTCCCCCGAGCCCCCTCTCCCAAATTTTTAAAG
CACTTTTTAGATTTTTTTTTCTCTTTCCTCCTTAAAAACAAAATTTATAT
ATAGATATATATATATATATAAATAATATACTTTTCCTCAGAGGAGCAGG
CAACAGTGTGGGATAAACAGAGTCACGATCAGAGGAACCCCAGGGTCTGG
TGATGGCAGGGATGGGGGGAGAGAGAGAAAATCCACAAATTCCAATGTCA
CAAAAGCAATAAAACAAACTAGAAAAAAAAAAGGTTTTACAAAATGAAAG
GAAGGAAAAAAAAAAAGGCAACCAACCACATTAGAAGTCTTGGCACTTTG
TAACGGAACGGGTACTACACTTTATCTTAATTCTTAATTTAAAAACATGT
TTACAAGTTACAACCAACTTCTATGAAAAGTTGAAAAGACAAAAAAAAAA
AAAAAAAGCGAGCGAGAGAGAGAGCGAGAGAGAGAGCGAGAGCAGAAGAA
ATTCCTAAAAGTCGATTTATTTTTGTACAAAATAATAAAAAAAAAAACCC
ACCACAAACGTAGAATCCACTTCTGTTCCCCAAAAAGCGAGAAGGGGGGT
TCAGGAGGAAGCCATCGCAGGGGACCTGGGAGACGCCCCGAGGTGTTTGT
GCTTCACCCCCAGACGTCAGCCTCGAAGGCAGGACTGTGGGGTGTTCGTG
CTGTGTTCCCCCCGCTCCCCCTTTCTGTCCCCTTTTTTGGTTCTGACGTG
AAGAGGTCTTAGCGCCCGCTTCTGTCCACGGGGTCTCTCCTTCCTCCTCC
CTAGCTCAGGGATGGGCCTTCCAGCCGGAGCACCCCGATCCCCATCCGGC
ACCCCCCAATCCCCCAACACGCCTGTCCCTCCCGCATGGCCACCAAGGAG
CTGGACCTTGGATGCGCCTACCCTGCTGAGGTGGGTGACAGGGGCCCCCC
ACCTCCAGGGCCTTAGAACCACCGCCCCTCTCCCCACCCCAGGCACCCCT
CTTTTTACTCAAAGGCACTGACTGTAATCCAGGGGGACTGGGACCTGCCT
CCCCCCAACCTCTGGCTCCCACAAGGCCCGGTGTTGACCGAGCCACAGGC
CACGGACAGGGGCCGGGGTTGGGGAGACTATGTCGCCAGATGCCAGGACG
CCCTCACCCCGTTTGCATATGCAATGCTAGCATGGGACCCCGAAAATAGA
CGCTCTGCTGCACTGAGACTTCTTGTCAATGCCCAACCGGCGGGGGGGTG
TCTCCCTGCCCCCGACCCCCCCATACCCCCTTCTCTGTGACACACACATC
TTCTCGTCTCTTTTTCTTTCATTGTTAAAGGGAAGCTTTTTAAGAAGGCA
ATTTTCATATTGTTTCTACAGGATGGTTTTGGTTCCCTTCCCTTCCCACC
CCCCCTTAAGCCTGTCAGCCCCCTCCAAATGTCTCAGGATCCCCCCTCTC
CCCTGGGGCTGGGTGACAGCACCCCGGCTGCGTTCACACCCCAGTGTCAC
AGGGCGAGCTGTTCTGGAGAGAAAACCATCTGTCGTGGCTGAGCGGGGAG
CTTGAACACCCAGGCCAGGGACACCCCTCCCCAGCTCCCAGAGAGGCCCC
CTGAGGGGTGAGCCCTCTTTCCACCTTCCCCTATCCATGCACCCCCTCGC
AATAAAACCAACTCTAAAATCACAGCTGTCGTCCTAGCCAGTGGGGGCGA
CCGGACTTGGGGGGTGGAGCCCTCTGGGACTTCCGTAGGAACAAGGGCTG
CGGCCCACCGCGACACTTACACAGACCTCGGGGATTGCACTAAACCCTCG
TTCCTAGCTCCGCACTCAGCTTCGCCTGTCCTGCCCGCCCACTTTGCCTT
AACTACCCGCCCGTCCTGGGGGCCACAGCCTCTGCATGGGCCCAGAGCCG
GGACCCCCCCAGCCCAGCCCCGCCCTCCCCAGACTCCGCGCAATCACATA
CTGTATATAGACGTGAATCGATTTTATTTTTATTCTTTAAATTAAGGTCG
TGATAAAGTGTTGCCAAAGATACCTGCTGAATTCTCGCGTTTCAGGAAAC
AAACAAACAAAAAAAAATGATATTTGAGGAGGGTCGTGTTGACTCCATAT
GAAAGGACACAGCTCAAAGCTTTTTTGTTTGGTTGTTTGGGGTTTTTTGT
GTTTTCTTTTTTTGGGGTGTTTTTTTTTTAACTGCCTGGTACAAAAAAAA
AAAGAGAAAAAAAAAAAAGAAAAACAATGCGAAATTGTTATTTCCATTCT
CATGGTGAAGTTGCGTGGACGCGTGTGTGCGTGTGTGCAAGAGAGCGGGA
GTGAGGTCCAGGCTGGGGTTGGGGGGCTTCAGGCGGGGGCGCCCGGGGGC
CGGGGAGGTGGCCGGGCCGGAGCCCCCGTCTGCAGTGCCCCCCAGCCTGC
CGGGCCCAGGAGAGAGAGAGAAGCATCTTTGCTACTAGCTGTTGCTGCTA
CCTGCCTCTGCCCCCCGACGCCCCCCGCCTTTTGAGATTAAGGAAAAAAA
AAAAAAGTCAAAAAAGTTTTTAAAAATGAAAAAAAAAAATTATAAACCAG
TGAATGTAAAATGCCGGAGCAGGCCCGGCCTGGCATGGGTGTGGACCTGC
AGCCAGGCAGGCTCGAGCGGGCGATACCAAAGTCTGCCCCCCCACCATTG
TGGCCATGCAGTCCTGTCACTGTCTTTTTGCTTCCTTCCGAGGGGGGTCC
CCCAGCCTCTTCCAGGGTCTTCCCCTGGAAGTGGGCGGCTGCAGGGAAGG
TGGGGGACAGGGGTCTTTGCACGATTCAGACCCCGGGGCCGTGGCAGGAG
CGGTCACCTCACAGGTGGTGACACTGAGGCAGGGGCCTCGGGGTGCCCCC
TCCCGCCCGGCAACCAGAATGGTTGGAGGCAAGACAGAGAGAATGAAAGG
AAAAACAGAAGAAAAAAAAATATTAAAAACCAACAAAAAAAGCAAAAATC
CTATTTTTTGAGAAAGAAAGATATTTATATTTGCAGTTTTATTTTAAAAA
GTTATTTAAGTTGAAGCAGCCTTCCTGGAGGTGGGGGGGGGGGGGTGGTG
GGTGGCTGGCGCAGGACGGGTCAGGGGCCTGGAGGCTGGGGGTGCCCCAG
GAGCTACAACCTCAGAGTTAAGACTAGCTCGCATTAAATACATAGATTTA
CGGGGGGGGGGGGGGGGGGCCGGGCCCAGGGGGTGGAGGGGGCCAGGGAG
ACCCCCATCCCTCGCCGGGGCTGCCTGGAGGCTGTGGACCAGGATCCGAT
GCCCAGGTCCCGCCCCCCACCCCACCCCAGGCCCAGAATCGAGGTGCCTT
GGACTTTGGAGGGGCCAGGCCTGGTGAATGGGGGGCGGGGCGGCGCCCTC
AGGGTACAGAGCACAGACAGATAGACATTCCAGAGACTGTATTGAGAGTC
TTTATAAAGTGTGGGAGATTTAAAAAAAAAAAAAACTGATAAAAATGCAC
TTTTTGGGAGTGGGGAGGGAGAAGCTTTAAAAGTAATAAAAAACAAACAA
AAACACAAAAGATGAAAAAACAAAAAAATTCATTTTTCTTGTACATAAAA
AAAAAAAAAGAACCACTAAACGCAGCCTGTTACGACC

An exemplary 584 amino acid polypeptide sequence of the ZBTB7A protein (UniProt 095365) is listed below as SEQ ID NO: 2:

MAGGVDGPIGIPFPDHSSDILSGLNEQRTQGLLCDVVILVEGREFPTHRS
VLAACSQYFKKLFTSGAVVDQQNVYEIDFVSAEALTALMDFAYTATLTVS
TANVGDILSAARLLEIPAVSHVCADLLDRQILAADAGADAGQLDLVDQID
QRNLLRAKEYLEFFQSNPMNSLPPAAAAAAASFPWSAFGASDDDLDATKE
AVAAAVAAVAAGDCNGLDFYGPGPPAERPPTGDGDEGDSNPGLWPERDED
APTGGLFPPPVAPPAATQNGHYGRGGEEEAASLSEAAPEPGDSPGFLSGA
AEGEDGDGPDVDGLAASTLLQQMMSSVGRAGAAAGDSDEESRADDKGVMD
YYLKYFSGAHDGDVYPAWSQKVEKKIRAKAFQKCPICEKVIQGAGKLPRH
IRTHTGEKPYECNICKVRFTRQDKLKVHMRKHTGEKPYLCQQCGAAFAHN
YDLKNHMRVHTGLRPYQCDSCCKTFVRSDHLHRHLKKDGCNGVPSRRGRK
PRVRGGAPDPSPGATATPGAPAQPSSPDARRNGQEKHFKDEDEDEDVASP
DGLGRLNVAGAGGGGDSGGGPGAATDGNFTAGLA

### Indications

Provided herein are methods and compositions for treating or preventing diseases or disorders associated with activity of the chromatin remodeling protein ZBTB7A. The disclosure provides methods of treating specific disorders with therapeutic compositions including inhibitors of ZBTB7A. The disclosure also provides inhibitors of ZBTB7A for use in methods of treating specific disorders with therapeutic compositions. In some embodiments, the diseases or disorders associated with activity of ZBTB7A are psychiatric disorders, neurological disorders, neuropsychiatric disorders, or mood disorders.

The methods used herein are useful for treating a subject in need thereof. As used herein, the terms “subject,” “patient,” and the like are used interchangeably. In some instances, the subject has a neurological disorder. In some instances, the subject has a psychiatric disorder. In some instances, the subject has a neuropsychiatric disorder. In some instances, the subject has a mood disorder. In some instances, the subject has a disorder affecting the central nervous system. In some instances, the subject has a disorder affecting the OFC. In some instances, the subject has a disorder related to neuroinflammation of the OFC. In some instances, the subject has, or is suspected to have, Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition.

### Major Depressive Disorder

In some embodiments, disclosed herein are methods of treating a subject exhibiting at least one symptom of, or determined to have, MDD. The subject may have one or more symptoms associated with MDD. These symptoms can include one or more of: feelings of sadness, tearfulness, emptiness or hopelessness; angry outbursts, irritability or frustration, even over small matters; loss of interest or pleasure in most or all normal activities, such as sex, hobbies or sports; sleep disturbances, including insomnia or sleeping too much; tiredness and lack of energy, so even small tasks take extra effort; reduced appetite and weight loss or increased cravings for food and weight gain; anxiety, agitation or restlessness; slowed thinking, speaking or body movements; feelings of worthlessness or guilt, fixating on past failures or self-blame; trouble thinking, concentrating, making decisions and remembering things; frequent or recurrent thoughts of death, suicidal thoughts, suicide attempts or suicide; unexplained physical problems, such as back pain or headaches; or any combination thereof.

Some embodiments provide a method of treating MDD in a subject in need thereof, comprising administering to the subject a therapeutically effective amount of an inhibitor of ZBTB7A; wherein the inhibitor of ZBTB7A reduces activity of ZBTB7A in cells of the subject.

### Bipolar Disorder

In some embodiments, disclosed herein are methods of treating a subject exhibiting at least one symptom of, or determined to have, Bipolar Disorder. The subject may have one or more symptoms associated with Bipolar Disorder. These symptoms can include manic and/or hypomanic episodes featuring one or more of being abnormally upbeat, jumpy or wired; increased activity, energy or agitation; exaggerated sense of well-being and self-confidence (euphoria); decreased need for sleep; unusual talkativeness; racing thoughts; distractibility; poor decision-making—for example, going on buying sprees, taking sexual risks or making foolish investments; or any combination thereof.

Some embodiments provide a method of treating Bipolar Disorder in a subject in need thereof, comprising administering to the subject a therapeutically effective amount of an inhibitor of ZBTB7A; wherein the inhibitor of ZBTB7A reduces activity of ZBTB7A in cells of the subject.

### Dysthymia

In some embodiments, disclosed herein are methods of treating a subject exhibiting at least one symptom of, or determined to have, dysthymia. The subject may have one or more symptoms associated with dysthymia. These symptoms can include one or more of sadness, emptiness or feeling down; loss of interest in daily activities; tiredness and lack of energy; low self-esteem, self-criticism or feeling of not being capable; trouble focusing clearly and trouble making decisions; problems getting things done well and on time; quickly becoming annoyed, impatient or angry; avoidance of social activities; feelings of guilt and worries over the past; poor appetite or overeating; sleep problems; hopelessness; or any combination thereof.

Some embodiments provide a method of treating dysthymia in a subject in need thereof, comprising administering to the subject a therapeutically effective amount of an inhibitor of ZBTB7A; wherein the inhibitor of ZBTB7A reduces activity of ZBTB7A in cells of the subject.

### Post-traumatic Stress Disorder

In some embodiments, disclosed herein are methods of treating a subject exhibiting at least one symptom of, or determined to have, PTSD. The subject may have one or more symptoms associated with PTSD. These symptoms can include one or more of recurrent, unwanted distressing memories of a traumatic event; reliving a traumatic event as if it were happening again (flashbacks); upsetting dreams or nightmares about a traumatic event, severe emotional distress or physical reactions to something that reminds the subject of a traumatic event; trying to avoid thinking or talking about a traumatic event; avoiding places, activities or people that remind the subject of a traumatic event; negative thoughts about self, other people, or the world; hopelessness about the future; memory problems, including not remembering important aspects of a traumatic event; difficulty maintaining close relationships; feeling detached from family and friends; lack of interest in activities the subject once enjoyed; difficulty experiencing positive emotions; feeling emotionally numb; being easily startled or frightened; always being on guard for danger; self-destructive behavior, such as drinking too much or driving too fast; trouble sleeping; trouble concentrating; irritability, angry outbursts or aggressive behavior; overwhelming guilt or shame; or any combination thereof.

Some embodiments provide a method of treating PTSD in a subject in need thereof, comprising administering to the subject a therapeutically effective amount of an inhibitor of ZBTB7A; wherein the inhibitor of ZBTB7A reduces activity of ZBTB7A in cells of the subject.

### Substance-Induced Mood Disorder

In some embodiments, disclosed herein are methods of treating a subject exhibiting at least one symptom of, or determined to have, Substance-induced mood disorder. The subject may have one or more symptoms associated with Substance-induced mood disorder. These symptoms can include one or more of pervasive sadness and/or hopelessness; feeling “empty”; changes in weight that include dramatic weight gain or loss in a short amount of time; unhealthy sleep patterns, such as sleeping too much or too little; lowered libido; frequent fatigue and low energy level; difficulty concentrating; feeling agitated or irritated; thoughts of death, including suicidal thoughts; or any combination thereof.

Some embodiments provide a method of treating Substance-induced mood disorder in a subject in need thereof, comprising administering to the subject a therapeutically effective amount of an inhibitor of ZBTB7A; wherein the inhibitor of ZBTB7A reduces activity of ZBTB7A in cells of the subject.

### Generalized Anxiety Disorder

In some embodiments, disclosed herein are methods of treating a subject exhibiting at least one symptom of, or determined to have, Generalized Anxiety Disorder. The subject may have one or more symptoms associated with Generalized Anxiety Disorder. These symptoms can include one or more of persistent worrying or anxiety about a number of areas that are out of proportion to the impact of the events; overthinking plans and solutions to all possible worst-case outcomes; perceiving situations and events as threatening, even when they aren't; difficulty handling uncertainty; indecisiveness and fear of making the wrong decision; inability to set aside or let go of a worry; inability to relax, feeling restless, and feeling keyed up or on edge; difficulty concentrating, or the feeling that your mind “goes blank”; fatigue; trouble sleeping; muscle tension or muscle aches; trembling, feeling twitchy; nervousness or being easily startled; sweating; nausea, diarrhea or irritable bowel syndrome; irritability; or any combination thereof.

Some embodiments provide a method of treating Generalized Anxiety Disorder in a subject in need thereof, comprising administering to the subject a therapeutically effective amount of an inhibitor of ZBTB7A; wherein the inhibitor of ZBTB7A reduces activity of ZBTB7A in cells of the subject.

### Social Phobia

In some embodiments, disclosed herein are methods of treating a subject exhibiting at least one symptom of, or determined to have, social phobia. The subject may have one or more symptoms associated with social phobia. These symptoms can include one or more of fear of situations in which the subject may be judged negatively; worry about embarrassing or humiliating oneself; intense fear of interacting or talking with strangers; fear that others will notice the subject looking anxious; fear of physical symptoms that may cause the subject embarrassment, such as blushing, sweating, trembling or having a shaky voice; avoidance of doing things or speaking to people out of fear of embarrassment; avoidance of situations where the subject might be the center of attention; anxiety in anticipation of a feared activity or event; intense fear or anxiety during social situations; analysis of one's performance and identification of flaws in one's interactions after a social situation; expectation of the worst possible consequences from a negative experience during a social situation; or any combination thereof.

Some embodiments provide a method of treating social phobia in a subject in need thereof, comprising administering to the subject a therapeutically effective amount of an inhibitor of ZBTB7A; wherein the inhibitor of ZBTB7A reduces activity of ZBTB7A in cells of the subject.

### Panic Disorder

In some embodiments, disclosed herein are methods of treating a subject exhibiting at least one symptom of, or determined to have, Panic Disorder. The subject may have one or more symptoms associated with Panic Disorder. These symptoms can include recurrent, unexpected panic attacks featuring one or more of sense of impending doom or danger; fear of loss of control or death; rapid, pounding heart rate; sweating; trembling or shaking; shortness of breath or tightness in one's throat; chills; hot flashes; nausea; abdominal cramping; chest pain; headache; dizziness, lightheadedness or faintness; numbness or tingling sensation; feeling of unreality or detachment; or any combination thereof.

Some embodiments provide a method of treating Panic Disorder in a subject in need thereof, comprising administering to the subject a therapeutically effective amount of an inhibitor of ZBTB7A; wherein the inhibitor of ZBTB7A reduces activity of ZBTB7A in cells of the subject.

### Mood Disorder Due to a General Medical Condition

In some embodiments, disclosed herein are methods of treating a subject exhibiting at least one symptom of, or determined to have, Mood Disorder Due to a General Medical Condition. The subject may have one or more symptoms associated with Mood Disorder Due to a General Medical Condition. These symptoms can include a prominent a persistent disturbance in mood that is judged to be a direct physiological consequence of a general medical condition, for example, Parkinson's disease, Huntington's disease, cardiovascular disease, metabolic conditions, endocrine conditions, autoimmune conditions, infections, certain cancers, or any combination thereof.

Some embodiments provide a method of treating Mood Disorder Due to a General Medical Condition in a subject in need thereof, comprising administering to the subject a therapeutically effective amount of an inhibitor of ZBTB7A; wherein the inhibitor of ZBTB7A reduces activity of ZBTB7A in cells of the subject.

### Methods of Treatment

### Antisense Oligonucleotides

In some embodiments, the pharmaceutical composition comprises an inhibitory nucleic acid, e.g., an antisense oligonucleotide that is complementary to one or more RNA transcripts that may encode a protein related to the disease in the subject. In some embodiments, as described in further detail below, the antisense oligonucleotide includes different modifications, e.g., in the sugar backbone to make it more cell permeable and nuclease resistant and physiologically non-toxic at low concentrations.

Inhibitory nucleic acids useful in the present methods and compositions include antisense oligonucleotides, modified bases/locked nucleic acids (LNAs), antagomirs, peptide nucleic acids (PNAs), double stranded RNA species, siRNAs, morpholinos, and other oligomeric compounds or oligonucleotide mimetics which hybridize to at least a portion of the target nucleic acid (e.g., an RNA transcript transcribed from the ZBTB7A gene) and modulate its abundance, splicing, post-transcriptional processing, or translation; see, e.g., U.S. Pat. Nos. 9,045,749 and 9,476,046. In some embodiments, the inhibitory nucleic acids include antisense RNA, antisense DNA, chimeric antisense oligonucleotides, antisense oligonucleotides comprising modified linkages, or combinations thereof. See, e.g., WO 2010040112.

In some embodiments, the inhibitory nucleic acids are 9 to 50, 9 to 21, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therein. In some embodiments, the antisense oligonucleotides are 15 nucleotides in length. In some embodiments, the antisense oligonucleotides are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29 or 30 nucleotides in length, or any range therein.

In some embodiments, the inhibitory nucleic acids are designed to target a specific region of an RNA transcript. For example, a specific functional region can be targeted, e.g., a region proximate to a transcription start site, or exon-intron-boundaries.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA: DNA or RNA: RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22:344-359; Nowotny et al., Cell, 121:1005-1016, 2005; Kurreck, European Journal of Biochemistry 270:1628-1644, 2003; Fluiter et al., Mol Biosyst. 5 (8): 838-43, 2009). In some embodiments, the oligonucleotide is a mixmer (includes alternating short stretches of LNA and DNA; Naguibneva et al., Biomed Pharmacother. 2006 November; 60 (9): 633-8; Ørom et al., Gene. 2006 May 10; 372 ( ) 137-41). Representative United States patents that teach the preparation of such hybrid structures comprise, but are not limited to, U.S. Pat. Nos. 5,013,830; 5,149,797; 5,220,007; 5,256, 775; 5,366,878; 5,403,711; 5,491, 133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2—NH—O—CH2, CH—N(CH3)—O—CH2 (known as a methylene(methylimino) or MMI backbone, CH2—O—N—(CH3)—CH2, CH2—N—(CH3)—N (CH3)—CH2 and O—N(CH3)—CH2—CH2 backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321, 131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41 (14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH3, OCH3, OCH3, O(CH2)nCH3, O(CH2)nNH2 or O(CH2)nCH3 where n is from 1 to about 10; Ci to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH2CH2OCH3, also known as 2′-O-(2-methoxyethyl)](Martin et al, Helv. Chim. Acta, 1995, 78, 486). Other preferred modifications include 2′-methoxy (2′-O—CH3), 2′-propoxy (2′—OCH2 CH2CH3) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, a palmityl moiety, or an octadecylamine or hexylamino-carbonyl-t oxycholesterol moiety.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention.

The inhibitory nucleic acids useful in the present compositions and methods are sufficiently complementary to all or part of a target RNA transcript, i.e., hybridize sufficiently well and with sufficient specificity, to give the desired therapeutic effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a target RNA transcript, then the bases are considered to be complementary to each other at that position. 100% complementarity is not required.

In the context of the present compositions and methods, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. The inhibitory nucleic acids and the target RNA transcript are complementary to each other when a sufficient number of corresponding positions in each molecule are occupied by nucleotides that can hydrogen bond with each other. Thus, “specifically hybridizable” and “complementary” are terms which are used to indicate a sufficient degree of complementarity or precise pairing such that stable and specific binding occurs between the inhibitory nucleic acid and the target RNA transcript. For example, if a base at one position of an inhibitory nucleic acid is capable of hydrogen bonding with a base at the corresponding position of the target RNA transcript, then the bases are considered to be complementary to each other at that position.

Although in some embodiments, 100% complementarity is desirable, it is understood in the art that a complementary nucleic acid sequence need not be 100% complementary to that of its target nucleic acid to be specifically hybridisable. A complementary nucleic acid sequence for purposes of the present compositions and methods is specifically hybridisable when binding of the sequence to the target RNA transcript interferes with the abundance, splicing, post-transcriptional processing, or translation of the RNA transcript, and there is a sufficient degree of complementarity to avoid non-specific binding of the sequence to non-target nucleic acid molecules under conditions in which specific binding is desired, e.g., under physiological conditions.

In general, the inhibitory nucleic acids useful in the compositions and methods described herein have at least 80% sequence complementarity to a target region within the target nucleic acid, e.g., 90%, 91%, 92%, 93%, 94%, 95%, 96%, 97%, 98%, 99% or 100% sequence complementarity to the target region within the mRNA transcribed from the ZBTB7A gene (e.g., a target region comprising the seed sequence). For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Antisense and other compounds of the invention that hybridize to a miR-128 target sequence are identified through routine experimentation. In general the inhibitory nucleic acids must retain specificity for their target, i.e., must not directly bind to, or directly significantly affect expression levels of, transcripts other than the intended target RNA transcript.

In some embodiments, the inhibitory nucleic acids are antisense oligonucleotides. Antisense oligonucleotides are typically designed to inhibit expression of a DNA or RNA target by binding to the target and halting expression at the level of transcription, affecting RNA stability, translation, or splicing. Antisense oligonucleotides of the present compositions and methods are complementary nucleic acid sequences designed to a target RNA transcript to achieve a therapeutic effect in a patient. Thus, oligonucleotides are chosen that are sufficiently complementary to the target, i.e., that hybridize sufficiently well and with sufficient specificity, to give the desired therapeutic effect.

In some embodiments, the ASO is capable of reducing the abundance of an RNA transcribed from the ZBTB7A gene. In some embodiments, the ASO is capable of reducing the abundance of a ZBTB7A RNA in a human cell. In some embodiments, the ZBTB7A RNA expression is reduced by at least about 30%, by at least about 35%, by at least about 40%, by at least about 45%, by at least about 50%, by at least about 55%, by at least about 60%, by at least about 65%, by at least about 70%, by at least about 75%, by at least about 80%, by at least about 85%, by at least about 90%, by at least about 95%, or about 100% compared to the ZBTB7A RNA abundance in a human cell that is not exposed to the ASO.

### RNA Interference

In some embodiments, the methods and compositions disclosed herein provide rAAV genomes comprising one or more AAV ITRs flanking a polynucleotide encoding one or more RNAs (including, but not limited to, small hairpin RNAs, antisense RNAs and/or microRNAs) that target ZBTB7A polynucleotides. The examples describe the use of exemplary rAAV encoding microRNAs (miRNAs). In the rAAV genomes, the miRNA-encoding polynucleotide can be operatively linked to transcriptional control DNA, specifically promoter DNA that is functional in target cells. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercially kits are available to produce, for example, custom siRNA molecules, such as SILENCER™ siRNA Construction Kit (Ambion Inc., Austin, TX) or psiRNA System (InvivoGen, San Diego, CA). In some embodiments, the rAAV genome comprises a DNA encoding an inhibitory RNA, the DNA comprising the sequence:

(SEQ ID NO: 3)
TGCTGATCATCAGCCGCCAGAATCTGGTTTTGGCCACTGACTGACCAG
ATTCTCGGCTGATGAT;
(SEQ ID NO: 4)
CCTGATCATCAGCCGAGAATCTGGTCAGTCAGTGGCCAAAACCAGATT
CTGGCGGCTGATGATC;
(SEQ ID NO: 5)
TGCTGTAGAAGTCCAAGCCATTGCAGGTTTTGGCCACTGACTGACCTG
CAATGTTGGACTTCTA;
(SEQ ID NO: 6)
CCTGTAGAAGTCCAACATTGCAGGTCAGTCAGTGGCCAAAACCTGCAA
TGGCTTGGACTTCTAC;
(SEQ ID NO: 7)
TGCTGATAGAAGTCCAAGCCATTGCAGTTTTGGCCACTGACTGACTGC
AATGGTGGACTTCTAT;
(SEQ ID NO: 8)
CCTGATAGAAGTCCACCATTGCAGTCAGTCAGTGGCCAAAACTGCAAT
GGCTTGGACTTCTATC;
(SEQ ID NO: 9)
TGCTGTGCACCTTCAGCTTGTCCTGTGTTTTGGCCACTGACTGACACAG
GACACTGAAGGTGCA;
or
(SEQ ID NO: 10)
CCTGTGCACCTTCAGTGTCCTGTGTCAGTCAGTGGCCAAAACACAGGA
CAAGCTGAAGGTGCAC.

In some embodiments, the rAAV genomes of the compositions and methods disclosed herein lack AAV rep and cap DNA. AAV DNA in the rAAV genomes (e.g., ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., J. Viral., 45:555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV -9 genome is provided in Gao et al., J. Viral., 78:6381-6388 (2004); the AAV-10 genome is provided in Mal. Ther., 13 (1): 67-76 (2006); and the AAV-11 genome is provided in Virology, 330 (2): 375-383 (2004). The AAVrh74 genome is provided in International Publication No. WO 2013/078316.

In some embodiments, the compositions and methods disclosed herein provide DNA plasmids comprising rAAV genomes. The DNA plasmids can be transferred to cells permissible for infection with a helper virus of AAV (e.g., adenovirus, El-deleted adenovirus or herpesvirus) for assembly of the rAAV genome into infectious viral particles. Techniques to produce rAAV particles, in which an AAV genome to be packaged, rep and cap genes, and helper virus functions can be provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10 and AAV-11. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example, US20050053922 and US20090202490, the disclosures of which are incorporated by reference herein in their entirety.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. Single-stranded rAAV are specifically contemplated. The foregoing documents are hereby incorporated by reference in their entirety herein, with particular emphasis on those sections of the documents relating to rAA V production.

In some embodiments, the compositions and methods disclosed herein comprise rAAV encoding inhibitory RNAs targeting the ZBTB7A gene. Compositions disclosed herein can comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods disclosed herein will vary depending, for example, on the particular rAAV, the mode of administration, the treatment goal, the individual, and the cell type(s) being targeted, and may be determined by methods standard in the art. Titers of rAAV may range from about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², aboutlx10¹³ to about 1×10¹⁴ or more DNase resistant particles (DRP) per ml. Dosages may also be expressed in units of viral genomes (vg). Dosages may also vary based on the timing of the administration to a human. These dosages of rAAV may range from about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, about 1×10¹⁶ or more viral genomes per kilogram body weight in an adult subject.

In some embodiments, provided herein are methods of transducing a target cell with a rAAV, in vivo or in vitro. The in vivo methods can comprise the step of administering an effective dose, or effective multiple doses, of a composition comprising a rAAV a subject (including a human being), in need thereof. If the dose is administered prior to onset/development of a disorder/disease, the administration is prophylactic. If the dose is administered after the onset/development of a disorder/disease, the administration is therapeutic. In some embodiments, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Examples of disorders contemplated for treatment with compositions and methods disclosed herein include Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition.

“Treatment” according to the methods disclosed herein thus alleviates (eliminates or reduces) at least one symptom associated with the disorder/disease state being treated, that slows or prevents progression to (onset/development) of a disorder/disease state, that slows or prevents progression of a disorder/disease state, that diminishes the extent of disease, and/or that results in remission (partial or total) of disease. Combination therapies are also contemplated by the methods disclosed herein. Combination as used herein includes both simultaneous treatment and sequential treatments. Combinations of methods of the invention with standard medical treatments (e.g., psychotherapy) are specifically contemplated, as are combinations with novel therapies.

Administration of an effective dose of the compositions may be by routes standard in the art including, but not limited to, systemic intramuscular, parenteral, intravenous, oral, buccal, nasal, pulmonary, intracranial, intrathecal, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the inhibitory RNAs targeting expression of the ZBTB7A gene. In some embodiments, the route of administration is systemic. In some, embodiments the route of administration is intrathecal. In some, embodiments the route of administration is introcerebroventricular. In some, embodiments the route of administration is cistema magna. In some, embodiments the route of administration is by lumbar puncture.

Transduction of cells with rAA V of the invention results in sustained expression of inhibitory RNAs targeting expression of the ZBTB7A gene. In another aspect, the present invention thus provides methods of administering/delivering rAAV which express inhibitory RNAs targeting expression of the ZBTB7A gene to a subject, preferably a human being. The term “transduction” is used to refer to the administration/delivery of inhibitory RNAs targeting expression of the ZBTB7A gene to a recipient cell either in vivo or in vitro, via a replication-deficient rAAV resulting in expression of an inhibitory RNA targeting expression of the ZBTB7A gene by the recipient cell.

Thus, the compositions and methods disclosed herein include administering an effective dose (or doses, administered essentially simultaneously or doses given at intervals) of rAAV that encode inhibitory RNAs targeting expression of the ZBTB7A gene to a subject in need thereof.

In another aspect, the compositions and methods disclosed herein provide methods of delivering a polynucleotide to the central nervous system of a subject in need thereof comprising intrathecal delivery of rAAV with a genome including the polynucleotide. In some embodiments, the rAAV genome is a self-complementary genome. In other embodiments, the rAAV genome is a single-stranded genome. In some embodiments, the rAAV is a rAAV9. In some embodiments, the rAAVis a rAAV2. In some embodiments, the rAAV is a rAAVrh74. In some embodiments, a non-ionic, low-osmolar contrast agent is also delivered to the subject, for example, iobitridol, iohexol, iomeprol, iopamidol, iopentol, iopromide, ioversol or ioxilan.

### Nucleic Acid-Guided Nucleases

In some embodiments, the disclosure provides a method of treating a psychiatric disorder, including but not limited to Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition, which includes administering a composition including one or more guide RNAs (gRNAs) targeting the ZBTB7A gene. In some embodiments, the gRNAs are administered to treat a psychiatric disorder. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP having a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the disclosure provides a method of inducing a double-stranded break (DSB) within the ZBTB7A gene including administering a composition having a guide RNA as described herein. In some embodiments, gRNAs are administered to recognize and bind to the ZBTB7A gene. The guide RNA is administered together with a nucleic acid (e.g., mRNA) or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease In some embodiments, a method of inducing a double-stranded break (DSB) within the ZBTB7A gene is provided comprising administering a composition comprising a guide RNA, such as a chemically modified guide RNA. In some embodiments, one or more sgRNAs or gRNAs are administered to induce a DSB in the ZBTB7A gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of modifying the ZBTB7A gene is provided comprising administering a composition comprising a guide RNA as described herein. In some embodiments, gRNAs or sgRNAs are administered to modify the ZBTB7A gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of treating a psychiatric disorder, including but not limited to Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition, is provided comprising administering a composition comprising a guide RNA as described herein. In some embodiments, gRNAs or sgRNAs are administered to treat the psychiatric disorder. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing expression levels of ZBTB7A in a subject including comprising administering a composition comprising a guide RNA as described herein. In some embodiments, gRNAs or sgRNAs are administered to reduce expression levels of ZBTB7A in a subject. The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the gRNA includes a guide sequence that is homologous or partially homologous to a portion of the ZBTB7A gene, together with an RNA-guided DNA nuclease such as a Cas nuclease translated from the nucleic acid induce DSBs, and non-homologous ending joining (NHEJ) during repair leads to a mutation in the ZBTB7A gene. In some embodiments, NHEJ leads to a deletion or insertion of a nucleotide(s), which induces a frameshift or nonsense mutation in the ZBTB7A gene.

In some embodiments, administering the guide RNA and nucleic acid encoding an RNA-guided DNA binding agent (e.g., in a composition provided herein) reduces levels (e.g., serum levels) of ZBTB7A in the subject.

In some embodiments, the use of one or more guide RNAs as described herein, (e.g., in a composition provided herein) and of a nucleic acid (e.g. mRNA) described herein encoding an nucleic acid-guided nuclease is provided for the preparation of a medicament for treating a human subject having a psychiatric disorder. The nucleic acid-guided nuclease may be a Cas9, e.g. an S. pyogenes Cas9. In particular embodiments, the guide RNA is chemically modified. In some embodiments, the composition that includes the guide RNA and nucleic acid is administered intravenously.

In some embodiments, a single administration of a composition that includes a guide RNA and nucleic acid provided herein is sufficient to knock down expression of the target protein, i.e., ZBTB7A. In some embodiments, a single administration of a composition that includes a guide RNA and nucleic acid provided herein is sufficient to knock out expression of the target protein in a population of cells. In other embodiments, more than one administration of a composition that includes a guide RNA and nucleic acid provided herein may be beneficial to maximize editing via cumulative effects. For example, a composition provided herein can be administered 2, 3, 4, 5, or more times, such as 2 times. Administrations can be separated by a period of time ranging from, e.g., 1 day to 2 years, such as 1 to 7 days, 7 to 14 days, 14 days to 30 days, 30 days to 60 days, 60 days to 120 days, 120 days to 183 days, 183 days to 274 days, 274 days to 366 days, or 366 days to 2 years.

In some embodiments, a composition is administered in an effective amount in the range of 0.01 to 10 mg/kg (mpk), e.g., 0.01 to 0.1 mpk, 0.1 to 0.3 mpk, 0.3 to 0.5 mpk, 0.5 to 1 mpk, 1 to 2 mpk, 2 to 3 mpk, 3 to 5 mpk, 5 to 10 mpk, or 0.1, 0.2, 0.3, 0.5, 1, 2, 3, 5, or 10 mpk. In some embodiments, a composition is administered in the amount of 2-4 mg/kg, such as 2.5-3.5 mg/kg. In some embodiments, a composition is administered in the amount of about 3 mg/kg. In some embodiments, treatment slows or halts disease progression. In some embodiments, efficacy of treatment is measured by improvement of the symptoms of the psychiatric disorder.

Some embodiments provide a method of treating a psychiatric disorder, neurological disorder, neuropsychiatric disorder, or mood disorder in a subject in need thereof, comprising administering to the subject a pharmaceutical composition described herein, wherein the psychiatric disorder, neurological disorder, neuropsychiatric disorder, or mood disorder is, for example, Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition.

Some embodiments provide a method of treating MDD, the method comprising administering to a subject in need thereof the pharmaceutical composition described herein.

Some embodiments provide a method of treating MDD in which the subject has been previously diagnosed with MDD.

Some embodiments provide a method of treating MDD in which the subject is currently suffering from MDD.

Some embodiments provide a method of treating Bipolar Disorder, the method comprising administering to a subject in need thereof the pharmaceutical composition described herein.

Some embodiments provide a method of treating Bipolar Disorder in which the subject has been previously diagnosed with Bipolar Disorder.

Some embodiments provide a method of treating Bipolar Disorder in which the subject is currently suffering from Bipolar Disorder.

Some embodiments provide a method of treating Dysthymia, the method comprising administering to a subject in need thereof the pharmaceutical composition described herein.

Some embodiments provide a method of treating Dysthymia in which the subject has been previously diagnosed with Dysthymia.

Some embodiments provide a method of treating Dysthymia in which the subject is currently suffering from Dysthymia.

Some embodiments provide a method of treating PTSD, the method comprising administering to a subject in need thereof the pharmaceutical composition described herein.

Some embodiments provide a method of treating PTSD in which the subject has been previously diagnosed with PTSD.

Some embodiments provide a method of treating PTSD in which the subject is currently suffering from PTSD.

Some embodiments provide a method of treating Substance-induced mood disorder, the method comprising administering to a subject in need thereof the pharmaceutical composition described herein.

Some embodiments provide a method of treating Substance-induced mood disorder in which the subject has been previously diagnosed with Substance-induced mood disorder.

Some embodiments provide a method of treating Substance-induced mood disorder in which the subject is currently suffering from Substance-induced mood disorder.

Some embodiments provide a method of treating Generalized Anxiety Disorder, the method comprising administering to a subject in need thereof the pharmaceutical composition described herein.

Some embodiments provide a method of treating Generalized Anxiety Disorder in which the subject has been previously diagnosed with Generalized Anxiety Disorder.

Some embodiments provide a method of treating Generalized Anxiety Disorder in which the subject is currently suffering from Generalized Anxiety Disorder.

Some embodiments provide a method of treating Social Phobia, the method comprising administering to a subject in need thereof the pharmaceutical composition described herein.

Some embodiments provide a method of treating Social Phobia in which the subject has been previously diagnosed with Social Phobia.

Some embodiments provide a method of treating Social Phobia in which the subject is currently suffering from Social Phobia.

Some embodiments provide a method of treating Panic Disorder, the method comprising administering to a subject in need thereof the pharmaceutical composition described herein.

Some embodiments provide a method of treating Panic Disorder in which the subject has been previously diagnosed with Panic Disorder.

Some embodiments provide a method of treating Panic Disorder in which the subject is currently suffering from Panic Disorder.

Some embodiments provide a method of treating Mood Disorder Due to a General Medical Condition, the method comprising administering to a subject in need thereof the pharmaceutical composition described herein.

Some embodiments provide a method of treating Mood Disorder Due to a General Medical Condition in which the subject has been previously diagnosed with Mood Disorder Due to a General Medical Condition.

Some embodiments provide a method of treating Mood Disorder Due to a General Medical Condition in which the subject is currently suffering from Mood Disorder Due to a General Medical Condition.

### EXAMPLES

The invention is further described in the following examples, which do not limit the scope of the invention described in the claims.

### METHODS

The following materials and methods were used in the following examples.

### Human Postmortem Samples

Postmortem human orbitofrontal cortex (Brodmann Area 11) tissues from 39 Caucasian subjects (20 cases, 19 controls) were obtained from the Human Brain Collection at University of Texas Southwestern (UTSW) (IRB approval for tissue banking at UTSW). Tissue preservation was achieved as previously described. Brains were placed on wet ice and transported to the UTSW Brain Bank facilities. Tissues were sliced, flash frozen in 2-methylbutane at −40° C., and stored in sections conserving anatomical landmarks at −80° C. OFC tissues were later sectioned from frozen slices. For each subject, the cause of death was determined by the Coroner Office, and toxicological screens were performed to obtain information on medication and illicit substance use at their time of death. The MDD group consisted of 20 (9 male and 11 female) individuals who met the Structured Clinical Interview for DSM-IV (Diagnostic and Statistical Manual of Mental Disorders-IV) Axis I Disorders: Clinician Version (SCID-I) criteria for Major Depressive Disorder. The control group comprised 19 subjects (12 male and 7 female Caucasians) with no history of MDD. Groups were matched for age, postmortem interval and RNA integrity number (RIN). For all subjects, psychological autopsies were performed, giving us access to detailed information on psychiatric and medical histories, as well as other relevant clinical and sociodemographic data.

### Fluorescence-Activated Nuclei Sorting (FANS) Sorting of Neuronal and Non-Neuronal Nuclei

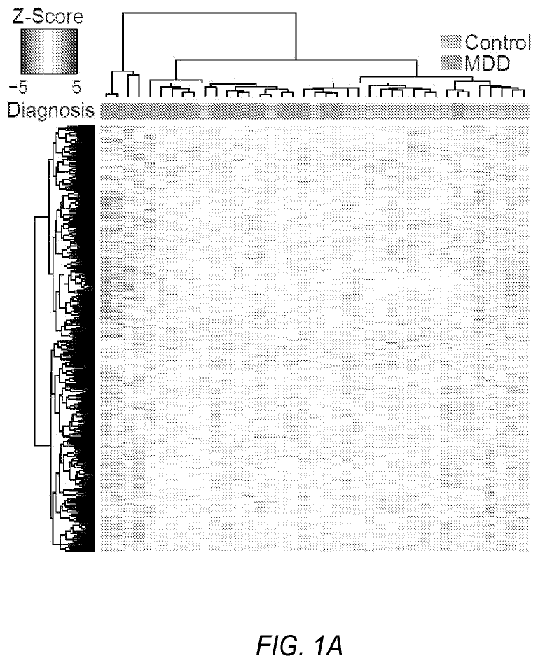

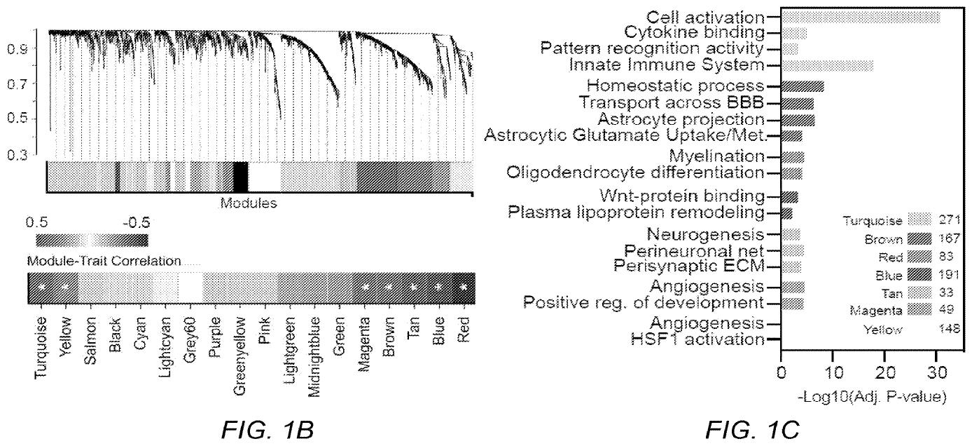

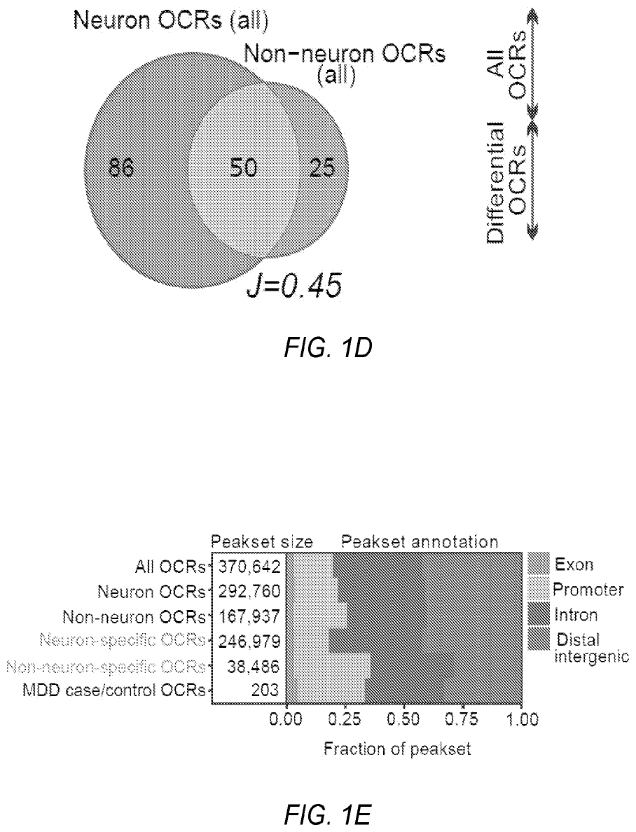

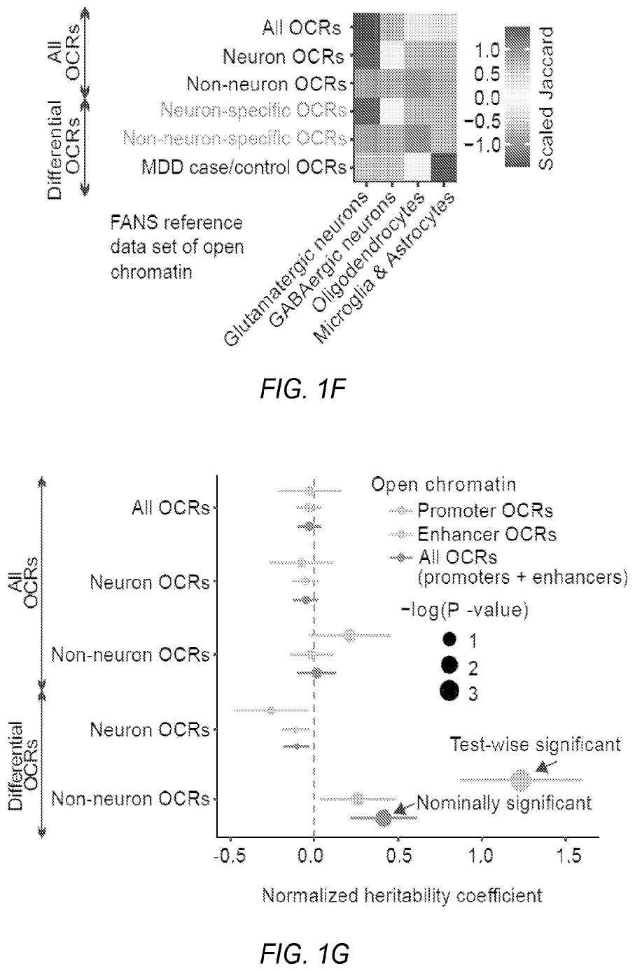

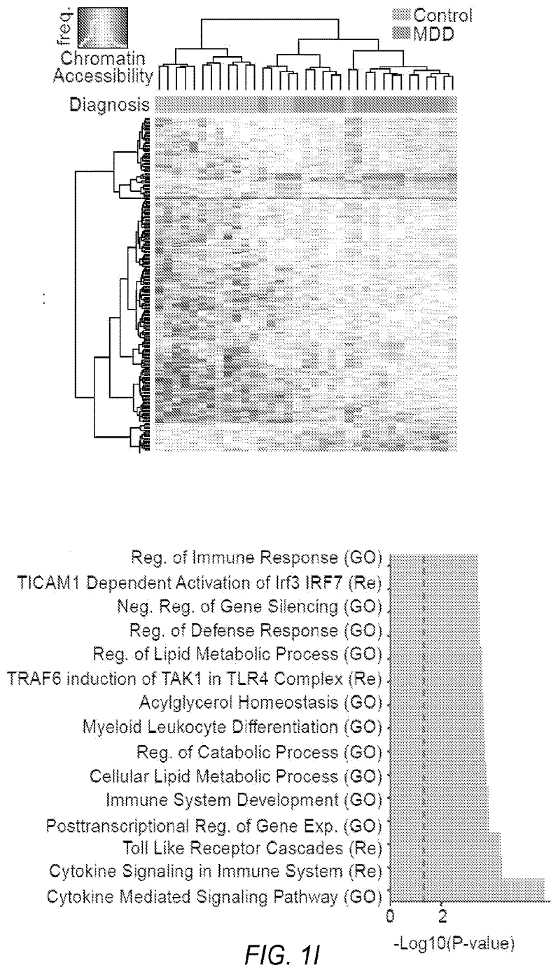

50 mg of frozen brain tissue was homogenized in cold lysis buffer (0.32M Sucrose, 5 mM CaCl₂), 3 mM Mg (Ace) 2, 0.1 mM, EDTA, 10 mM Tris-HCl, pH8, 1 mM DTT, 0.1% Triton X-100) and filtered through a 40 μm cell strainer. The flow-through was underlaid with sucrose solution (1.8 M Sucrose, 3 mM Mg (Ace) 2, 1 mM DTT, 10 mM Tris-HCl, pH8) and subjected to ultracentrifugation at 24,000 rpm for 1 hour at 4° C. Pellets were thoroughly resuspended in 500 μl DPBS and incubated in BSA (final concentration 0.1%) and anti-NeuN antibody (1:1000, Alexa488 conjugated, Millipore) under rotation for 1 hour, at 4° C., in the dark. Prior to FANS sorting, DAPI (Thermoscientific) was added to a final concentration of 1 μg/ml. DAPI positive neuronal (NeuN+) and non-neuronal (NeuN-)nuclei were sorted into tubes pre-coated with 5% BSA using a BD-FACSAria flow cytometer (BD Biosciences) equipped with a 70 μm nozzle (Supplemental FIG. 1). 39 tissue dissections from 1 brain region were subjected to FANS, resulting in 78 (39 NeuN− and 39 NeuN+) distinct nuclear populations.

### RNA-sequencing

For human postmortem OFC, ~25 mg pre-sectioned flash-frozen tissue was utilized for RNA extraction. For mouse studies, animals were euthanized, and brains were removed whole and flash frozen (for bulk sequencing), or processed fresh for cell-type specific isolation with magnetically-activated cell sorting (MACs). Brains were sectioned at 100 μm on a cryostat (bulk) or brain block (MACs), and GFP was illuminated using a NIGHTSEA BlueStar flashlight to microdissect virally infected tissues with a 2 mm punch. For both human and mouse experiments, OFC tissues were homogenized in Trizol (Thermo Fisher), and RNA was isolated on RNeasy Minelute Microcolumns (Qiagen) following manufacturer's instructions. Following elution, samples were enriched for mRNA via polyA tail selection beads, and mRNA libraries were prepared using the Illumina Truseq RNA Library Prep Kit V2 (#RS-122-2001). Libraries were pooled and sequenced on the Illumina Novaseq platform, with an average read count of approximately 20 million paired-end reads per sample. RNA-seq data was pre-processed and analyzed as previously described. Briefly, FastQC (Version 0.72) was performed on the concatenated replicate raw sequencing reads from each library to ensure minimal PCR duplication and sequencing quality. Reads were aligned to the hg38 or mouse mm 10 genome using HISAT2 (Version 2.1.0) and annotated against Ensembl v90. Multiple-aligned reads were removed, and remaining transcript reads were counted using featurecounts (Version 2.0.1). For mouse RNA-sequencing experiments with multiple groups, RUVg56 was performed to normalize read counts based on empirically determined control genes that do not vary between control and stress groups (i.e. genes with p-val>0.5 based on a first-pass differential expression analysis performed prior to RUVg normalization). For human RNA-seq and mouse RNA-seq experiments with two groups, RUVr56 was performed to normalized read counts based on the residuals from a first-pass GLM regression of the unnormalized counts on the covariates of interest. DESEQ257 (Version 2.11.40.6) was used to perform pairwise differential expression analyses between indicated comparisons. Differentially expressed (DE) genes were defined at FDR<0.1. Threshold free Rank-Rank Hypergeometric Overlap (RRHO) maps were generated to visualize transcriptome-wide gene expression concordance patterns as previously described, using RRHO2 (Version 1.0). For RRHO comparing ATAC-seq vs. RNA-seq, signed log p-value from the RNA-seq DESEQ2 output was ranked for each transcript that was also associated with a differentially accessible peak in the ATAC-seq.

For the human MDD dataset, we used the WGCNA package (Version 1.71) to construct the co-expression network for the top 2000 most variable genes in the set. We chose a suitable soft threshold power of 7 for scale-free network construction with the function pickSoftThreshold. The resulting gene co-expression network was visualized as the heatmap based on dissimilarity of TOM with hierarchical clustering dendrogram, and the number of genes in each module was counted. The correlation between modules and the trait of MDD was assessed by the Pearson correlation coefficients, with students t-test, and a p value of <0.05 was considered statistically significant. Gene ontology (GO) enrichment analysis was performed for genes in each significant module (and for GO analyses on DE genes from other experiments) with idep (for GO, Reactome, and TRANSFAC/JASPAR databases) with total detected genes as background, and enrichR (for cell-type and human disease databases) to test for overrepresented gene categories in our list of DE genes. FDR for representative GO terms from the top 20 terms is calculated based on nominal P-value from the hypergeometric test.

Gene Set Enrichment Analysis was performed using the EGSEA package (Version 1.10.0) against EGSEA's gene set database including, GO, MsigDB, Hallmark, GeneSetReg and GeneSetDB, and Kegg. Results from twelve algorithms were used to calculate collective gene set scores, and gene sets were ranked by adjusted p-value. Bar graph displays representative (and shared) gene sets from the top 10 most significant sets, plotted according to −Log (adj. p-value) (X-axis), and the direction (LogFC) of regulation. Odds Ratio analyses were carried out on DE gene lists using the GeneOverlap R package version 1.26.059.

### Generation of Human ATAC-Seq Libraries

ATAC-seq reactions were performed using an established protocol with minor modifications. Following FANS, 50,000 sorted nuclei were centrifuged at 500×g for 10 min, 4° C. Pellets were resuspended in transposase reaction mix (25 μL 2× TD Buffer (Illumina Cat #FC-121-1030) 2.5 μL Tn5 Transposase (Illumina Cat #FC-121-1030) and 22.5 μL Nuclease Free H2O) on ice. Samples were incubated at 37° C. for 30 min and then purified using the MinElute Reaction Cleanup kit (Qiagen Cat #28204) according to the manufacturer's instructions. Following purification, library fragments were amplified using the Nextera index kit (Illumina Cat #FC-121-1011), under the following cycling conditions: 72° C. for 5 minutes, 98° C. for 30 seconds, followed by thermocycling at 98° C. for 10 seconds, 63° C. for 30 seconds, and 72° C. for 1 minute for a total of 5 cycles. In order to prevent saturation due to over-amplification, a 5 μl aliquot was then removed and subjected to qPCR for 20 cycles to calculate the optimal number of cycles needed for the remaining 45 μL reaction. The additional number of cycles was determined as follows: (1) Plot linear Rn vs. Cycle (2) Calculate the # of cycles that corresponds to ¼ of maximum fluorescent intensity. In general, we found adding 4-6 cycles to this estimate yielded optimal ATAC-seq libraries, as determined by analysis on Bioanalyzer High Sensitivity DNA Chips (Agilent technologies Cat #5067-4626). Libraries were amplified for a total of 13-19 cycles. Following PCR, ATAC-seq libraries were resolved on 2% agarose gels and fragments ranging in size from 100 bp-1Kbp were excised and purified (Qiagen Minelute Gel Extraction Kit-Qiagen Cat #28604). Libraries were quantified by quantitative PCR (KAPA Biosystems Cat #KK4873) prior to sequencing. Libraries were sequenced on Hi-Seq2500 (Illumina) obtaining 2×50 paired-end reads. After quality controls (see below), 70 ATAC-seq libraries were retained for downstream analysis.

### Data Processing

A summary of the data processing pipeline is provided in FIGS. 8A-8B. The preprocessing of ATAC-seq samples involved the following steps:

### Alignment

Raw sequencing reads were generated by the sequencing center demuxed and with adaptors trimmed. FASTQ files were linked to the sample clinical and demographics metadata based on pooling ID's and barcodes. Reads were subsequently aligned to the hg19 reference genome with the pseudoautosomal region masked on chromosome Y with the STAR aligner (v2.5.0) 61, using the following parameters:-alignIntronMax 1,-outFilterMismatchNmax 100,-alignEndsType EndToEnd,-outFilterScoreMinOverLread 0.3,-outFilterMatchNminOverLread 0.3. Having a coordinate-sorted BAM, we further excluded reads that: (1) were mapped to more than one locus using samtools; (2) were duplicated using PICARD; and (3) mapped to the mitochondrial genome.

### Genotype Calling

Genotypes were called by GATK (v3.5.0). We performed: (1) indel-realignment; (2) base score recalibration; and (3) joint genotype calling across all samples for variants having a phred-scaled confidence threshold≥10. We excluded clustered variants, variants in ENCODE blacklisted regions64, and variants not present in dbSNP v14665. Genotype concordance between samples was assessed using both the kinship coefficient calculated by KING v1.966 and the fraction of concordant genotype calls. For these analyses, we kept only variants with minor allele frequencies (MAF)≥25%. The two approaches yielded comparable results, with both indicating a clear and unambiguous separation of samples. Using this approach, we were able to confirm that neuronal and non-neuronal libraries supposedly originating from the same subject showed markedly higher genotype concordance score compared to the comparison with unrelated samples (FIG. 71).

### Sex Determination of Samples

The sex of the samples was assessed using three metrics: (1) the heterozygosity rate of chromosome X genotype calls outside the pseudoautosomal regions. For this, we removed variants with MAF<5%. A high heterozygosity rate can indicate contamination in male samples. (2) The read counts of OCRs adjacent to FIRRE and XIST genes that are predominantly expressed in females. (3) Read counts in OCRs on chromosome Y outside the pseudoautosomal region. Using this approach, we detected and excluded two samples that were supposed to originate from a male subject but they were genetically females. After their removal, all remaining samples matched the expected sex characteristics (FIG. 7H).

### Quality Control of ATAC-Seq Samples

For each sample, we calculated the following metrics: (1) total number of initial reads; (2)number of uniquely mapped reads; (3) fraction of reads that were uniquely mapped and additional metrics from the STAR aligner; (4) Picard duplication and insert metrics; (5) rate of reads mapped to the mitochondrial genome; (6) PCR bottleneck coefficient (PBC), which is an approximate measure of library complexity estimated as (non-redundant, uniquely mapped reads)/(uniquely mapped reads); (7)normalized strand cross-correlation coefficient (NSC) and relative strand cross-correlation coefficient (RSC), which are metrics that use cross-correlation of stranded read density profiles to measure enrichment independently of peak calling; (8) fraction of reads in peaks (FRiP), which is the fraction of reads that fall in detected peaks (see below for peak calling) and similarly the fraction of reads in only blacklisted peaks and the ratio between these two metrics. On average, we obtained more than 27 million uniquely mapped paired-end reads per sample. The rate of reads that mapped to the mitochondrial genome was below 2% since we generated ATAC-seq libraries using FANS separated nuclei, instead of whole cells. The bigWig tracks for each sample were manually inspected. A total of six libraries were excluded, having failed QC (including sex check) and/or visual inspection in IGV, leaving 70 libraries that were subjected to further analysis.

### Peak Calling and Read Quantification

First, we merged the BAM-files of samples of the same diagnosis and cell type and subsampled to a uniform depth of, at most, 454 million paired-end reads. We subsequently created bigWig files and called peaks using these merged bam files and created a joint set of peaks requiring each peak to be called in at least one of the merged BAM-files. Peaks for OCRs were called by MACS (v2.1) 67, using the following parameters68:-keep-dup all-shift-100-extsize 200-nomodel. After removing peaks overlapping the blacklisted genomic regions, 371,820 peaks remained. Next, we counted how many reads for each sample overlapped consensus peaks using the featureCounts function in RSubread69 (v.1.15.0). We counted fragments (defined from paired-end reads), instead of individual reads. This resulted in a sample by peak matrix of read counts, obtained using the following parameters: allowMultiOverlap=F, isPairedEnd=T, strandSpecific=0, requireBothEndsMapped=F, minFragLength=0, maxFragLength=2000, checkFragLength=T, countMultiMappingReads=F, countChimericFragments=F.

### Differential Analysis of Chromatin Accessibility

We performed a statistical analysis of chromatin accessibility to detect genomic regions with significant differences in chromatin structure among neuronal and non-neuronal cells.

First, we used the sample-by-peak read count matrix (70 samples by 371,820 OCRs). We subsequently excluded 1,178 OCRs using a criteria of “CPM≥1 in at least 10% of the samples”, resulting in our final sample-by-peak read count matrix (70 samples by 370,642 OCRs). From here, we applied the trimmed mean of M-values (TMM) 70 to normalize the read count followed by quantile normalization to achieve a balanced distribution of reads across samples of the same cell type.

Covariate exploration: Next, we tested whether we can find biological or technical sample-level covariates that affect the observed read count. For these covariates (e.g. number of peaks called in the sample, FRiP, chrM metrics, RSC and NSC, and Picard insert metrics), we normalized to the median of the cell. All 63 covariates were then tested for inclusion in differential analysis as detailed in the following: As a starting point for building the model to explain chromatin accessibility in the peaks, we selected cell type by diagnosis (2×2=4 levels) and sex (2 levels). To select additional covariates, we sought a good “average model” of chromatin accessibility over all OCRs. For each additional tested covariate, we asked how many OCRs showed an improved Bayesian Information Criterion (BIC) score minus how many showed a worse BIC score when the covariate was added to the “base” linear regression model. Here, we required that at least 5% of the OCRs showed a change of 4 in the BIC score, corresponding to “positive” evidence against the null hypothesis71. However, no covariate satisfied the BIC score criteria for inclusion. We were unable to find any covariate even after adjusting the threshold of minimal BIC (tested values={2, 4, 10}) and/or minimal fraction of OCRs exceeding this threshold (tested values={2%, 5%}). Overall, our final model included 2 variables (cell type by diagnosis [4] and sex [2]), where the number of levels for factor variables is noted here in square brackets. This model accounted for 5 DF.

Differential analysis: We used the voom WithQuality Weights function from the limma package72 to model the normalized read counts. Then, we performed differential chromatin accessibility analysis by fitting weighted least-squares linear regression models for the effect of each variable on the right-hand side on accessibility of each OCR:

$$chromatin accessibility ∼ cell type : diagnosis + Sex + ( 1 \vert Person\_ID )$$

Validation of differential OCRs: To validate the relevance of differential OCRs, we applied the following strategies: permutation test and machine-learning test. For the former one, we randomly permuted MDD case/control status (n=100 permuted datasets) and performed differential analysis using the same setting as for primary analysis. We measured (i) whether the sets of differential OCRs on permuted datasets are smaller compared to non-permuted datasets and (ii) whether the P-value rankings of differential OCRs on non-permuted datasets are close to normal distribution. For machine learning validation, we trained six machine learning models for prediction of MDD case/control status built on the reported set of (i) differential OCRs and (ii) the same number of randomly selected OCRs. We applied the repeated 5-fold cross-validation (krepeat=10) and, additionally, we repeated the whole process 10 times with different sets of randomly selected OCRs. Then, we measured an improvement of prediction performance of the classifier based on differential OCRs over classifiers utilizing random OCRs. The following machine learning methods were tested, using the default setting in R-package73: Naïve Bayes (nb), Random forest (rf), Nearest neighbor (knn), Logistic regression (multinom), SVM with linear kernel (svmLinear), and SVM with polynomial kernel (svmPoly).

### Annotation of OCRs and Gene Set Enrichment Analysis

We determined the genomic context per each OCR based on its proximity to the closest gene as assigned by ChIPSeeker74. For this, we created a transcript database using GenomicFeatures75 and Ensembl genes. The genomic context was defined as promoter (+/−3 kb of any TSS), 5′-UTR, 3′-UTR, exon, intron, and distal intergenic. We used GREAT approach20 to assign OCRs to genes and perform enrichment analysis with combined set of Gene Ontology76, biological processes with the curated canonical pathways from REACTOME77, KEGG78, and PID79, all accessed from MSigDB 6.080. We further pruned highly similar gene sets by iteratively removing those with a Jaccard index≥0.5, preferentially keeping the bigger gene set. This resulted in 4,590 gene sets (biological processes and pathways).

Overlap of OCRs with Common Variants in MDD

To determine whether the sets of neuronal, non-neuronal, and consensual OCRs as well as differential OCRs are enriched for common MDD GWAS variants19, we calculated partitioned heritability using LD-sc18. This analysis assesses if common genetic variants in the genomic regions of interest explain more of the heritability for a given trait than genetic variants not overlapping the genomic regions of interest, normalized by the number of variants in either category. The algorithm allows for correction of the general genetic context of the annotation using a baseline model of broad genomic annotations (like coding, intronic, and conserved regions). By using this baseline model, the algorithm focuses on enrichments above those expected from the general genetic context of the interrogated regions. We excluded the broad MHC-region (chr6: 25-35 MB) and, otherwise, used default parameters.

### Motif Matching

In order to identify candidates for DNA-binding proteins with recognition motifs enriched in our MDD-specific OCR set, we utilized the RSAT suite peak-motifs, a computational pipeline that discovers motifs in input sequences, and compares them with position-specific scoring matrix (PSSM) transcription factor databases. Input sequences are scanned to predict binding sites, and the background model is a Markov chain of order 2 trained on the input sequences. Using peak-motifs, word-based analysis was first performed on the MDD-specific OCR set (n=183 sequences) with hexanucleotides (k=6) and heptanucleotides (k=7). The tool combines four pattern-discovery algorithms that utilize overrepresentation and positional bias as two criteria to detect significant oligonucleotide, which are then used as seeds to build probabilistic description of motifs (PSSMs), indicating residue variability at each position of the motif. Discovered motifs were compared with the JASPAR nonredundant core database of known transcription factor binding motifs to predict associated transcription factors (using compare-matrices). Several metrics are computed to measure the similarity between each matrix pair (including Pearson correlation, width normalized correlation). These metrics are converted to ranks, and a mean rank is computed to enable comparison between candidate factors. The peak-motifs pipeline discovered a motif (FIG. 2a) that was significantly enriched in MDD-specific OCR sequences. The distribution of this motif within OCR sequences is shown in FIG. 2a, indicating a relatively higher number of sites near sequence centers. For the top 5 candidate transcription factors identified as matches to this motif, the bar graph in Extended Data FIG. 7a displays the consensus score from the Human Protein Atlas for expression in human brain for each factor. The mRNA expression data is derived from deep sequencing of RNA (RNA-seq) from 37 different normal tissue types.

In order to characterize the functional role for the discovered motif from the peak-motifs pipeline, we utilized GOMo (v5.3.3), from the MEME-suite of tools (FIG. 2B). This approach calculates associations between a user-specified DNA regulatory motif [expressed as a position weight matrix (PWM)] and Gene Ontology (GO) terms, by computing an association score between the (putative) targets of the input TF motif and each GO term in the GO map. An empirically generated p-value for the enrichment of the GO term is also computed for the association score for each GO term with respect to the motif, based on the rank sum test null model.

### Footprinting Analysis

To determine the bound/unbound status of transcription factors in neuronal and non-neuronal cells as well as in MDD cases and controls, we performed footprinting analysis using TOBIAS (v. 0.12.4). Following the settings from our previous study, we searched for the presence of 431 motifs representing 798 transcription factors (some motifs are shared due to their high similarity) in consensus OCRs of four merged BAM files representing both cell types & MDD diagnosis status. First, we ran the TOBIAS module ATACorrect to correct for Tn5 insertion bias in input BAM files, followed by TOBIAS ScoreBigwig to calculate footprinting scores across OCRs. Then, TOBIAS BINDetect combined footprinting scores with the information of transcription factor binding motifs to evaluate the individual binding positions of each transcription factor and determine whether a given position was bound by a given transcription factor or not for each condition, i.e. cell type and brain region. Finally, TOBIAS PlotAggregate was used to visually compare the aggregated footprints for select motifs.

### QPCR

In order to measure mRNA gene expression for gene targets of interest, FAN-sorted nuclei (FIG. 11D) from human postmortem OFC tissues were prepared as described above, with addition of RNAse inhibitor in the sorting collection buffer, and pelleted for RNA extraction. Cultured human primary astrocytes (HPA) were washed with sterile PBS, scraped and pelleted for RNA extraction. For both nuclei samples and HPA cell samples, pellets were resuspended in RLT lysis buffer with 10% B-mercaptoethanol (B-ME), homogenized with a 22g needle and syringe, combined with equal volume 70% ethanol, and applied to Qiagen micro minelute column. RNA was washed, treated with DNAase, and eluted in 13 μl of RNAse-free water, according to manufacturer's instructions.

To measure ZBTB7A in bulk brain tissues, postmortem human OFC tissues were sectioned into 50 mg sections. Frozen mouse brains were sliced into 1 mm coronal slices in a brain matrix, and 2 mm OFC punches were removed. For both human and mouse tissues, sections were homogenized in Trizol (Thermo Fisher #15596026) with a motorized pestle, followed by chloroform extraction and precipitation with 70% ethanol. Samples were applied to a Qiagen micro minelute column, and RNA was washed, treated with DNAse, and eluted according to manufacturer's instructions into 13 μl RNAse-free water. For all RNA samples (derived from nuclei, cells, or brain tissues), 500 ng of total RNA was utilized to synthesize cDNA using the Bio-Rad script cDNA synthesis kit (#1708891). From this reaction, 4 ng of cDNA was used to perform qPCR with PowerUp™ SYBR™ Green Master Mix (#A25742), according to the manufacturer's instructions. Target gene CT values were averaged over 3 replicates, normalized to the reference gene (human brain-HPRT1, mouse brain-Gapdh), and the AACT was calculated. Graphs show experimental group fold change relative to controls, mean±/−SEM.

### Western Blot

In order to measure protein expression, postmortem human OFC tissues were sectioned into 50 mg sections. Frozen mouse brains were sliced into 1 mm coronal slices, and 2 mm OFC punches were removed. For both human and mouse tissues, sections were homogenized in 200 ml RIPA cell lysis buffer, 1× protease inhibitor cocktail and 1× phospho-stop inhibitor using a 1 ml dounce homogenizer. Following homogenization, lysates were briefly sonicated with a probe sonicator for five 1s pulses. Protein concentrations were measured using the DC protein assay kit (BioRad), and 20 μg of protein was loaded onto 4-12% NuPage BisTris gels (Invitrogen) for electrophoresis. Proteins were then fast-transferred to nitrocellulose membranes and blocked for 1 hr in 5% milk in PBS+0.1% Tween 20 (PBST), followed by incubation with primary antibodies overnight at 4° C. with rotation. The following antibodies were used: monoclonal rabbit anti-zbtb7a (Abcam #ab175918) (1:1000) for human blots, rabbit anti-Zbtb7a (Abcam #ab106592) (1:1000) for mouse blots, as well as rabbit anti-Gapdh (Abcam #ab9485) (1:10,000), and rabbit anti-H3.3 (Abcam #ab1791). After overnight primary antibody incubation, membranes were washed 3× in PBST (10 min) and incubated for 1 hr with horseradish peroxidase conjugated anti-rabbit (BioRad 170-6515, lot #: 64033820) secondary antibodies (1:10000; 1:50000 for anti-Gapdh antibody, BioRad) in 5% milk/PBST at RT. After three final washes with PBST, bands were detected using enhanced chemiluminescence (ECL; Millipore). Densitometry was used to quantify protein bands using ImageJ Software (NIH). Target protein measurements were normalized to Gapdh bands, and experimental group fold change was calculated relative to controls.

### Animals

C57BL/6J mice were purchased from The Jackson Laboratory (Stock #024694). All procedures were done in accordance with NIH guidelines and the Institutional Animal Care and Use Committees of the Icahn School of Medicine at Mount Sinai.

### Male Chronic Social Defeat Stress Paradigm

In order to investigate the expression of Zbtb7a in the context of a mouse model of stress, the Chronic Social Defeat Stress (CSDS) in males was performed as described previously. Briefly, a cohort of 20 8-week old male C57BL/6J mice were randomly assigned to either the control or stress condition. Animals in the stress group underwent 10 consecutive days of a single 7-minute defeat session with an unfamiliar CD1 retired breeder male that had been previously screened for aggression towards C57BL/6J mice. Following the defeat session, the C57 mice spent 24 hours in the same cage as the CD1, separated by a perforated divider to allow for sensory contact. Control animals spent 24 hours in the same cage as a different male C57BL/6J for each day of the 10 day paradigm, separated by a perforated divider. The Social Interaction (SI) test was performed as described previously. Briefly, in the first trial, the subject mouse was allowed to freely explore an arena with an empty mesh cage inside an interaction zone. In the second trial, a CD1 target was put in the mesh cage, and the mouse was again allowed to explore the arena. Trials are recorded and scored by Ethovision software: SI ratio score was calculated as (time spent in interaction with target)/(time spent in interaction zone without target). Control mice typically have scores≥1.0, indicating increased time spent investigating the unfamiliar mouse. In the stress mice, scores<1.0 are defined as “avoidant” and mice are described as stress susceptible, while scores>1.0 are defined as “non avoidant” and the mice are described as stress resilient. In a typical CSDS experiment, approximately 30% of WT mice will segregate into the stress resilient group.

### Viral Constructs

Briefly, ZBTB7A overexpression plasmids (Origene Cat. #RC222759) were cloned into either a Lentiviral CMV-driven construct for use in cell culture experiments (shown in FIG. 11) or a GFAP-GFP Adeno-associated virus (AAV) construct (Addgene plasmid #50473) for use in animal experiments (utilized in FIG. 4 and FIG. 5). Lentiviral vectors contained either a ZBTB7A-HA tagged overexpression construct or an empty vector expressing RFP. AAV vectors contained either an ZBTB7A overexpression construct or GFP. For AAV vectors utilized in FIG. 3, a miRNA targeting endogenous Zbtb7a was generated using the BLOCK-iT™ Pol II miR RNAi Expression Vector Kit with EmGFP (Thermo #K493600), in addition to a scramble negative control (Thermo #K493600) (miR-neg) which forms a hairpin structure just as a regular pre-miRNA, but does not target any known vertebrate gene. Constructs were packaged into GFAP driven AAV expression vectors to generate AAV-GFAP-Zbtb7a-miR-GFP and AAV-GFAP-mir-neg-GFP. Purified plasmids were sent to GENEWIZ for sequence validation. Plasmids were sent to Cyagen Biosciences for packaging into Lentivirus or AAV6 serotype viruses at high titer (>10\hat{ }12 units).

Negative control sequence without 5′ overhangs:

(SEQ ID NO: 11)
GAAATGTACTGCGCGTGGAGACGTTTTGGCCACTGACTGACGTCTCCACG
CAGTACATTT

Oligos used for Zbtb7a KD:

NM_010731.3_1062_top:
(SEQ ID NO: 5)
TGCTGTAGAAGTCCAAGCCATTGCAGGTTTTGGCCACTGACTGACCTGCA
ATGTTGGACTTCTA
NM_010731.3_1062_bottom:
(SEQ ID NO: 6)
CCTGTAGAAGTCCAACATTGCAGGTCAGTCAGTGGCCAAAACCTGCAATG
GCTTGGACTTCTAC

### Primary Human Astrocyte Cell Culture and LPS Treatment

Primary Human Astrocytes isolated from human cerebral cortex and frozen at first passage were purchased from Sciencell (#1800) and cultured in Astrocyte medium (Sciencell #1801) on 50 ug/ml coated Matrigel-coated plates (BD #354230). Cells were treated with lentivirus particles at MOI=~2 to overexpress ZBTB7A or RFP. Approximately 72 hours after lentivirus transduction, PHAs were treated with 2 ug puromycin to positively select for cells expressing each construct. After 6 days of selection, cells were collected for molecular analyses (FIGS. 11F-11H). For testing ZBTB7A mRNA expression in inflammatory conditions, the cells were treated with either saline or LPS (Sigma Cat. #L2630) at 1 μg/ml for 8 hours, and collected for molecular analyses (FIG. 11I).

### Primary Mouse Astrocytes

Primary astrocytes were cultured from frontal cortical dissections of mouse pups at P1, as previously described. Briefly, cortices were dissociated, and diluted in 10% Fetal Bovine Serum (OmegaSci, FB-11)/1% penicillin-streptomycin in DMEM (Gibco, 11995-065) and plated at a density of one brain per uncoated T75 flask. On DIV 1, plates were tapped to dislodge neurons and the media was changed to remove floating cells. Remaining astrocytes were maintained and grown to confluency and seeded at a density of approximately 3×106 cells/plate for subsequent experiments. Once confluent, the cells were treated with either saline or LPS (Sigma Cat. #L2630), and collected for molecular analyses (FIG. 11J).

### TRAP-Sequencing Data

Polyribosome immunoprecipitation was performed as described. Briefly, mice were put through the CSDS paradigm, as described above, and sacrificed by rapid decapitation. Brain regions were dissected. Brain tissue was homogenized and homogenates were centrifuged to remove cell debris, and NP-40 (EMD Biosciences) and DHPC (Avanti Polar Lipids) were added, followed by another centrifugation step. The supernatant, which contains the ribosomes, was subjected to immunoprecipitation using anti-EGFP antibodies conjugated to Protein G magnetic Dynabeads (Invitrogen). The beads were washed, and RNA was extracted using Trizol reagent following the manufacturer's protocol. RNA was further purified on RNeasy columns (Qiagen). RNA was amplified using the Ovation RNA-seq System V2 (NuGEN). Library preparation and amplification was performed by the Rockefeller University Genomic Facility, and libraries were sequenced on the Illumina HiSeq platform.

### Immunohistochemistry

Mice were anesthetized with intraperitoneal (i.p.) injection of ketamine/xylazine (10/1 mg/kg), and then perfused transcardially with ice cold phosphate buffered saline (PBS) followed by ice cold 4% paraformaldehyde (PFA) in PBS. Next, brains were post-fixed in 4% PFA overnight at 4° C. and then transferred into 30% sucrose in PBS for two days. Brains were then cut into serial 40 μm coronal slices in a cryostat at −20C. Free floating slices containing OFC were washed 3× in tris buffered saline (TBS), incubated for 30 min in 0.2% Triton-X in TBS to permeabilize tissue, and then incubated for 1 hr at RT in blocking buffer (0.3% Triton-X, 3% donkey serum in TBS). Brain slices were then incubated overnight on an orbital rotator at 4 degrees C. with primary antibodies. 24 hours later, brain slices were washed 3× in TBS and then incubated for 2 hrs at room temperature (RT) with a fluorescent-tagged AlexaFluor 680 secondary antibody. Brain sections were then washed 3× in TBS, incubated with DAPI (1:10000, lot #: RK2297251, Thermo Scientific 62248) for 5 min at RT, mounted on Superfrost Plus slides (Fischer Scientific) and then coverslipped with Prolong Gold (Invitrogen). Immunofluorescence was visualized using a confocal microscope (Zeiss LSM 780). For quantification of Zbtb 7a overlap with Gfap, images were split into respective color channels, and we calculated the Mander's Correlation Coefficient89 using the coloc2 package (version 2.0.2) on FIJI, which performs pixel intensity correlation and statistical testing.

### Animal Surgeries

Male C57BL/6J mice were anesthetized with a ketamine/xylazine solution (10/1 mg/kg) i.p., positioned in a stereotaxic frame (Kopf instruments) and 1 μl of viral construct was infused bilaterally into the OFC using the following coordinates; AP, 2.6 mm; ML, ±1.2 mm; V, 2.8 mm, angle) 10°. Following surgery, mice received meloxicam (1 mg/kg) s.c. and topical antibiotic treatments for 3 days. All behavioral testing or electrophysiological recordings commenced 21 days after surgery to allow for maximal expression of the viral constructs.

### Magnetic Cell Sorting

For magnetic-activated cell sorting, we collected virally-infected fresh OFC tissues, pooling 3 mice per n, and performed the MACs protocol, following manufacturer's instructions. Briefly, OFC tissues were removed and washed in cold D-PBS, and tissue was dissociated using the Adult Brain Dissociation Kit, mouse and rat (Miltenyi #130-107-677) enzyme kit in combination with the gentleMACs Octo Dissociator with Heaters (Miltenyi #130-096-427). Samples were strained with MACs SmartStrainers (70 μM, Miltenyi #130-098-462), and spun at 300 g for 10 minutes at 4C. Myelin debris was removed using myelin removal beads (Miltenyi #130-096-733) in combination with the autoMACs Pro Separator. Samples were magnetically labeled with Anti-ACSA-2 Microbeads (Miltenyi #130-097-678) to isolate astrocytes with the autoMACs Pro Separator with the positive selection program. The negative fraction was subsequently incubated with Adult Non-neuronal Cell biotin-antibody cocktail (Miltenyi #130-126-603), followed by anti-biotin microbeads (Miltenyi #130-126-603) and then processed on the autoMACs Pro Separator to isolate neuronal cells via negative selection. For validation experiments in FIGS. 12A-12E, and FIGS. 14A-14B, negative fractions following astrocyte isolation were further processed with anti-Cd11b microbeads (Miltenyi #130-093-634) to isolate microglia, followed by incubation with anti-Pdgfra microbeads (Miltenyi #130-094-543) to isolate immature oligodendrocytes, using the autoMACS Pro Separator prior to isolation of neuronal fraction, as described above. Isolated astrocyte and neuronal cells were then counted, with 50K cells separated for ATAC-seq, and the remainder of cells used for RNA extraction via trizol, followed by cleanup using the Qiagen Minelute kit. For the mouse ATAC-seq, MACs-isolated cells were processed according to the OMNI-ATAC protocol, which has been optimized for fresh cells.

### Mouse ATAC-seq Differential Accessibility Analysis:

Raw sequencing reads were aligned to the mouse genome (mm 10) using default settings of HISAT2. Only uniquely mapped reads were retained. Alignments were filtered using SAMtoolsv1.19 to remove duplicate reads. Peak calling was performed using MACSv2.1.124 with settings-nomodel-shift-100-extsize 200. Peaks were filtered for FDR<0.05. Differential analyses were performed using diffReps20 with a window size of 1 kb. A default p-value cutoff of 0.0001 was used. Peaks and differential sites were further annotated to nearby genes or intergenic regions using the region analysis tool from the diffReps package.

### Reward Sensitivity Tasks and Operant Saccharin Behavior:

Animals were single housed and given restricted access to water (4 h/day for 4d) before the start of the behavioral training. During the course of the experiment, mice were given access to water for 2 h each day (post-session).

The first stage of the experiment was four days of Pavlovian cue-reward association training for reinforcement with 0.2% saccharin-solution. Modular standard mouse operant chambers enclosed in light and sound blocking cubicles were used, equipped with white house lights and ventilation fans-interior dimensions: 55.69×38.1×40.64 cm; exterior dimensions: 63.5×43.18×44.45 cm; walls: 1.9 cm) (MedAssociates, Fairfax, VT). Each chamber contained two retractable levers and one central reward magazine containing a dipper calibrated to provide ~50 μl of liquid saccharin reward per each reinforcement. Each daily session was 40-min (with operant levers retracted), in which mice learned to introduce their noses into the central reward magazine to get saccharin rewards, which were delivered every 60 s. A cue light above the magazine signaled reward delivery. Correct and incorrect saccharin retrieval was detected via infrared beam breaks upon head entry in the magazine and automatically recorded by MedPC software.

Next, mice were put through 7 days of 1 h sessions of operant learning training, in which mice were conditioned to lever press on a fixed-ratio 1 (FR1) schedule for ad libitum saccharin reinforcement. The basic settings were: session onset was indicated by illumination of the house light, and extension of both active and inactive levers; one active lever response (FR1) initiated magazine-cue light illumination and subsequent reward delivery, and following retrieval a 2.5 s inter-trial interval (ITI) was initiated; the session terminated after 1 hr.

In overexpression studies, after lever-press training as described above, mice were further trained on a reversal learning paradigm, using two levers positioned left and right of the central liquid reward magazine. For the baseline phase, mice went through 1 session/day for 8 days of training: On FRI, a response at the correct lever initiated magazine light and reward delivery, the session terminated after 30 min. At the reversal phase, the previously incorrect lever was now correct and vice versa, so that non-reward-shift behavior was required; reversal testing lasted for an additional 8 days.

### Subthreshold Social Defeat Paradigm

In order to investigate the role of Zbtb7a in stress vulnerability, we performed the Subthreshold variant of the Social Defeat Paradigm (SSDS) on a cohort of 8 week old C57BL/6J male mice that were injected with either the rAAV6-GFAP-Zbtb7a OE construct or the rAAV6-GFAP-GFP empty control vector into the OFC 3 weeks previously. Half of each virus group was randomly assigned to the stress group or control group. The stress group underwent the SSDS paradigm as described previously43. Briefly, the stress mice were subjected to three 5-min defeat sessions with an aggressive CD1 male mouse consecutively on a single day, separated by a 15-minute rest period. The experimental mouse then spent 24 hours in the aggressor home cage, separated by a perforated divider to allow sensory exposure to the aggressor, and was then tested for social interaction as described above. Note that WT mice do not show behavioral deficits after the SSDS paradigm.

### Sucrose Preference

48 hours after the SI test, we tested the SSDS cohort for sucrose preference using a two-bottle choice paradigm. For two days before the test, animals were habituated to drinking from two bottles. At the start of the sucrose test, bottles were filled with either 1% sucrose or water. The liquid weight of each bottle was weighed each day. Preference was calculated as a percentage of sucrose consumed over total liquid volume consumed percentage [100×volume of sucrose consumed (in bottle A)/total volume consumed (bottles A and B)].

### Forced Swim

Mice were placed in a 4 liter glass beaker with 2L of room-temperature water for 7 minutes. Each session was recorded and scored by a blinded observer to record the number of seconds each mouse was immobile during the last 4 minutes of the test.

### Singe-Cell Suspension Preparation and Flow Cytometry

Briefly, virally-transduced OFC tissue was dissected, minced and digested with 450 U/ml collagenase I, 125 U/ml collagenase XI, 60 U/ml DNase I and 60 U/ml hyaluronidase (Sigma) in PBS for 40 min at 37° C. Samples were passed through a 70-μm cell strainer and mixed with 30% percoll layered on top of 70% percoll. The percoll gradient was centrifuged at 500 g for 30 min with the brake off. The cell fraction was collected and washed with PBS before downstream applications. Total viable cell numbers were quantified using counting beads (Thermo Fisher Scientific). Cell suspensions were stained with the antibody cocktail in PBS supplemented with 2% FBS and 0.5% BSA. The following monoclonal antibodies were used for flow cytometry analyses at a dilution of 1/700: anti-CD45 (BioLegend, clone 30-F11, 103147), anti-CD11b (BioLegend, clone M1/70, 101226), anti-CD11c (Biolegend, clone N418, 117333), anti-TREM2 (R&D Systems, clone 237920, FAB17291P), anti-P2RY12 (Biolegend, clone S16007D, 848003), anti-ASCA2 (Miltenyi Biotec, clone REA969, 130-116-245), anti-MHCII (BioLegend, clone M5/114.152, 107602) and anti-CCR2 (R&D systems, clone 475301, MAB55381). Viable cells were identified through negative staining for Zombie NIR (BioLegend). Data were acquired on a Cytek Aurora and analyzed with FlowJo (Tree Star). Flow cytometry gating strategy shown in Supplementary FIG. 3 included all cells, singlets, live cells and cell populations were identified as astrocytes (ACSA2+CD45−) or microglia (CD45midP2RY12+CD11b+).

### Electrophysiology

Male C57BL/6J mice (age approximately 60 days) were deeply anesthetized with isoflurane and then decapitated, followed by rapid removal and chilling of the brain. Coronal slices (300 μm thick) were prepared using a Compresstome vibrating microtome (Precisionary, Natick, MA), in ice-cold sucrose cutting solution (in mM: 215 sucrose, 2.5 KCl, 1.6 Na2HPO4, 26 NaHCO₃, 4 MgSO4, 1 CaCl2 and 20 glucose). The slices then were transferred to artificial cerebrospinal fluid (ACSF; in mM: 120 NaCl, 3.3 KCl, 1.2 NaHPO4, 26 NaHCO₃, 1 MgSO4, 2 CaCl2 and 11 glucose; pH 7.2, 300 mOsM; bubbled with 95% 02/5% CO2) at 32° C. for 30 minutes, after which they were transferred to room temperature ACSF and allowed to recover for at least one hour. Recordings were obtained in a submersion recording chamber superfused with ACSF (1 mL/min) at room temperature. A concentric bipolar stimulating electrode was placed in layer 1 of the orbital frontal cortex to evoke synaptic responses using a 100 μs stimulus delivered by an IsoFlex stimulus isolator (AMPI, Jerusalem, Israel). A glass Ag/AgCl electrode filled with ACSF recorded field excitatory synaptic potentials (fEPSPs) from layer 5. Recordings were acquired using Axoclamp 2A and Axopatch 1D amplifiers, Digidata 1440A analog-digital convertor, and pClamp software 10 (all from Molecular Devices, San Jose, CA). Signals were low-pass filtered at 2 kHz and digitized at 10 kHz. An input-output (I-O) curve was constructed by recording fEPSPs in response to stimuli ranging from 100-800 uA (average of three fEPSPs per stimulus strength, recorded at intervals of 20 seconds between stimuli, starting with the lowest intensity). Given the proximity of the recording electrode to the stimulating electrode within respective layers of the OFC, we plotted peak amplitude (instead of peak slope), to avoid effects of recording artifacts. Following construction of an I-O curve, the stimulus intensity that evoked a fEPSP of ~50% of maximum amplitude was used in rundown experiments. Separate slices from the same animals were used for the rundown experiments. For rundown experiments, a single 30-s train was delivered at 10 Hz after establishment of a stable baseline. The percentage change in fEPSP amplitude from baseline was calculated. All data were graphed as means±SEM.

### Calcium Imaging

Calcium imaging was performed in 2D primary mixed cultures of mouse cortical neurons and glia (including astrocytes) to assess neuronal and astrocytic activity, using the genetically encoded calcium indicator GCaMP6f. Primary mixed cultures were transduced with AAV1-hSyn-GCaMP6f (Addgene #100837-AAV1) or AAV5-gfaABCID-cyto-GCaMP6fto (Addgene #52925-AAV5) to ensure selective expression solely in neurons or in astrocytes, respectively, at least 5 days prior to the imaging sessions. Both GCaMP6f-astrocyte and GCaMP6f-neuronal cultures were treated with the AAV-GFAP-ZBTB7A overexpression (OE) vector for ZBT-OE conditions. In GCaMP6f-neuron cultures, to control for the astrocyte AAV treatment, “control virus” conditions were additionally treated with an AAV5-RFP empty vector. Neurons and astrocytes activity were imaged independently after 14-23 DIV, in mixed cultures plated on poly-d-lysine matrix (0.1 mg/mL, gibco #A38904-01) coated 10 mm glass coverslips. A Nikon Eclipse TE2000-U microscope with a 10× objective was used to image the coverslips with the mixed primary cultures mounted on a diamond-shaped chamber. To excite and detect GCaMP6f fluorescence, a 480 nm LED (Mic-LED-480A, Prizmatix), a HQ480/40× excitation filter, a Q505LP dichroic mirror, and a HQ535/50m emission filter (Semrock) were used. Emitted fluorescence was projected onto a sCMOS Zyla chamber camera (VSC-01910, Andor) and sampled at 8.87 fps for GCaMP6f-expressing neurons (284×240 pixels, 3×3 binning) and 4.7 fps for GCaMP6f-expressing astrocytes (160×135 pixels, 4×4 binning). Nikon Elements software (NIS-Elements AR 5.20.01) was used to control light source and sCMOS camera.

To record spontaneous neuronal or astrocytic calcium spikes, mixed cultures were continuously perfused during fluorescence recording with artificial cerebrospinal fluid buffer (ACSF), with the following composition (in mM): NaCl 125, KCl 5, D-Glucose 10, HEPES-Na 10, CaCl2 3.1, MgCl2 1.3. (pH adjusted to 7.4 with HCl and osmolarity corrected with sucrose to 290-300 mOsm). Perfusion was gravity fed (flow rate of 0.065 ml/s) and controlled with a ValveBank8 II (AutoMate Scientific Inc.).

GCaMP6f-expressing neurons or astrocytes ROIs were segmented, and raw fluorescence data were background corrected and extracted using Nikon Elements software. ΔF/F was calculated as (Ft-Fmin)/Fmin, being Ft=raw fluorescence at time t, and Fmin=minimum fluorescence for the entire trace. A low-pass Butterworth filter was used to denoise the ΔF/F trace, and an adaptive iteratively reweighted Penalized Least Squares (AirPLS) based algorithm96 was applied to baseline correct the ΔF/F trace for drift, using R-Studio (R version 4.0.3). Spike detection was performed using a custom script in R that applied specific criteria for neuronal and astrocytic calcium events. For neurons, action potential-derived Ca2+spikes had the following criteria (framerate acquisition of 8.87 fps): duration<45 frames, rise phase>=3 frames, fall phase>=3 frames, rise phase<=fall phase, peak height>4*SD (for ROIs with SD<20), and peak height>3*max background signal. For astrocytes, Ca2+events detected fell in the following criteria (framerate acquisition of 4.7 fps): duration<100 frames, rise phase>=10 frames, fall phase>=10 frames, peak height>4*SD (for ROIs with SD<20), and peak height>4*max background signal. Final n of cells per condition was as follows: astrocyte-GCaMP6f (n=623 cells control virus saline, n=559 cells ZBT OE saline, n=747 cells control virus LPS, and n=517 cells ZBT OE LPS) and neuron gCaMP6f (n=135 cells control virus saline, n=1277 cells ZBT OE saline, n=238 cells control virus LPS, and n=1324 cells ZBT OE LPS). Statistical analysis was performed in GraphPad Prism 8.4.3.

### Chemogenetic Manipulation

In order to determine if neuronal hyperexcitability contributes to the observed behavioral effects of ZBTB7A OE, we performed the Subthreshold variant of the Social Defeat Paradigm (SSDS) on a cohort of 8 week old C57BL/6J male mice that were injected with the pAAV-hSyn-hM4D (Gi)-mCherry to express the inhibitory Gi DREADD (Addgene #50475-AAV2), in combination with either the rAAV6-GFAP-Zbtb7a OE construct or the rAAV6-GFAP-GFP empty control vector into the OFC 3 weeks previously. Half of each virus group was randomly assigned to the ZBT OE or GFP viral group. Both viral groups underwent the SSDS paradigm as described previously. The experimental mouse then spent 24 hours in the aggressor home cage, separated by a perforated divider to allow sensory exposure to the aggressor. The mice were then single housed for 24 hours, and then half of each viral group was injected with either the DREADD agonist Deschloroclozapine (Tocris #7193) at 1 ug/kg in 1% DMSO or vehicle. Fifteen to twenty minutes post-injection, the mice were tested for social interaction as described above.

### Example 1: Identifying Regulatory Signatures of Major Depressive Disorder (Mdd) in Chromatin

In order to define gene expression changes in MDD orbitofrontal cortex (OFC), bulk RNA-sequencing was performed on postmortem OFC tissues from 20 MDD cases versus 19 matched healthy controls (FIGS. 1A, 6A-6C). For human postmortem OFC, ~25 mg pre-sectioned flash-frozen tissue was utilized for RNA extraction. OFC tissues were homogenized in Trizol (Thermo Fisher), and RNA was isolated on RNeasy Minelute Microcolumns (Qiagen) following manufacturer's instructions. Following elution, samples were enriched for mRNA via polyA tail selection beads, and mRNA libraries were prepared using the Illumina® Truseq® RNA Library Prep Kit V2 (#RS-122-2001). Libraries were pooled and sequenced on the Illumina® Novaseq™ platform, with an average read count of approximately 20 million paired-end reads per sample. RNA-seq data was pre-processed and analyzed. FastQC (Version 0.72) was performed on the concatenated replicate raw sequencing reads from each library to ensure minimal PCR duplication and sequencing quality. Reads were aligned to the hg38 genome using HISAT2 (Version 2.1.0) and annotated against Ensembl v90. Multiple-aligned reads were removed, and remaining transcript reads were counted using featurecounts (Version 2.0.1). RUVr was performed to normalized read counts based on the residuals from a first-pass GLM regression of the unnormalized counts on the covariates of interest. DESEQ2 (Version 2.11.40.6) was used to perform pairwise differential expression analyses between indicated comparisons. Differentially expressed (DE) genes were defined at FDR<0.1. Threshold free Rank-Rank Hypergeometric Overlap (RRHO) maps were generated to visualize transcriptome-wide gene expression concordance patterns using RRHO2 (Version 1.0).

The WGCNA package (Version 1.71) was used to construct the co-expression network for the top 2000 most variable genes in the set. We chose a suitable soft threshold power of 7 for scale-free network construction with the function pickSoftThreshold. The resulting gene co-expression network was visualized as the heatmap based on dissimilarity of TOM with hierarchical clustering dendrogram, and the number of genes in each module was counted. The correlation between modules and the trait of MDD was assessed by the Pearson correlation coefficients, with Students t-test, and a p value of <0.05 was considered statistically significant. Gene ontology (GO) enrichment analysis was performed for genes in each significant module (and for GO analyses on DE genes from other experiments) with idep (for GO, Reactome, and TRANSFAC/JASPAR databases) with total detected genes as background, and enrichR (for cell-type and human disease databases) to test for overrepresented gene categories in our list of DE genes. FDR for representative GO terms from the top 20 terms is calculated based on nominal P-value from the hypergeometric test.

Differential gene expression analysis and weighted gene correlation network analysis (WGCNA) implicated glial cell function and inflammatory response as the most altered pathways in MDD vs. controls, suggesting a key role for non-neuronal cell dysregulation in this region (FIGS. 1B-1C, 6D-6F). In order to assess distinct patterns of chromatin accessibility in neuronal and non-neuronal (primarily glial)nuclei of human MDD OFC, FANS (Fluorescence-Activated Nuclear Sorting) was implemented coupled with ATAC-seq (Assay for Transposase-Accessible Chromatin followed by Sequencing) on nuclear preparations obtained from 20 MDD cases vs. 19 matched healthy controls. Extensive quality control assessments of the ATAC-seq libraries were performed to yield a total of 70 high quality sample libraries (FIGS. 7A-9C). To define the regulatory programs that specify each cell population, active Open Chromatin Regions (OCRs) were identified in neuronal and non-neuronal samples, accounting for 4.79% and 2.65% of the genome, respectively (FIG. 1D). Neuronal OCRs were found to be more distal to transcription start sites (TSSs) compared to non-neuronal OCRs, reflecting a more complex regulatory scheme and higher levels of functional diversity among neuronal subtypes (FIGS. 1E, 7B).

In order to determine whether OCRs exhibit cell-type enrichment, a curated dataset of cell-type specific markers for OCRs across four cell types was used (FIG. 1F). Neuronal and non-neuronal OCRs displayed robust overlaps with respective cell-type-specific open chromatin reference datasets (FIG. 1F). The majority of genetic variants that influence human disease are located within non-coding regulatory regions of the genome. Therefore, in order to investigate overlaps between identified OCRs and common risk variants in MDD, the heritability coefficient for each set of OCRs was calculated, stratified by genomic context (FIG. 1G). Enrichment of MDD-associated genetic variants was observed only in non-neuronal-specific promoter OCRs (FIG. 1G). These findings indicate that active regulatory elements within non-neuronal OFC cells are relevant to the genetic risk for affective disorders. Furthermore, in agreement with the RNA-seq analysis (FIG. 1C), gene targets of these non-neuronal-specific promoter OCRs (LogFC>1.5, within 3000 bp of TSS) overlapped most significantly with astrocyte-specific gene sets, indicating a possible role for astrocyte dysfunction in MDD-related traits (FIG. 10A).

Differential OCRs were assessed in each nuclei population for specificity to MDD-diagnosis. Consistent with both the RNA-seq analysis and the data indicating that non-neuronal OCRs are enriched for common risk variants in MDD GWAS, MDD-associated changes were detected in chromatin accessibility only in non-neuronal OCRs (203° C. Rs, FIG. 1H) but not in neuronal OCRs. Gene targets associated with MDD-specific OCRs displayed significant expression changes in FAN-sorted non-neuronal nuclei from MDD vs. control subjects, and correlations between MDD-OCRs and bulk RNA-seq signatures were observed from this same patient cohort (FIGS. 10B-10C). Finally, in order to characterize the biological processes regulated by MDD-specific OCRs in glia, gene set enrichment analysis (GSEA) was performed, which revealed significant changes in pathways associated with cellular activation, including NF-kB inflammation, cytokine-mediated cascades, and toll-like receptor signaling, as well as astrocyte-related processes such as cholesterol metabolism and vascular regulation (FIGS. 11, 10D). Together, these data demonstrate that MDD-specific OCRs contain regulatory elements that mediate cellular stress responses that are disrupted in MDD, and converge with evidence that glial inflammatory stress plays a role in the pathophysiology of MDD, particularly in OFC.

### Example 2: Zbtb7A Identified as a Transcription Factor Regulating Mdd-Specific Open Chromatin Regions

To identify potential transcriptional regulators of MDD-specific OCRs in non-neuronal cells, transcription factor (TF) motif discovery analysis was implemented. A motif was identified that was robustly enriched in non-neuronal OCRs that showed significantly higher chromatin accessibility in MDD cases (57 motif occurrences out of 183 OCRs; FIG. 2A). In order to characterize the functional role of this enriched regulatory motif, gene ontology (GO) analysis was performed, which revealed significant association between this enriched motif and gene targets involved in the regulation of inflammatory response, cytokine pathways, and NF-κB cascades (FIG. 2B), confirming that this enriched motif is involved in the same regulatory processes that are enriched in MDD-specific OCRs (FIG. 1I). The top five candidate TFs with binding motifs that matched the enriched motif sequence were identified (FIG. 2C). In human OFC postmortem tissues, only two of these candidate TFs were expressed at detectable levels in human brain (FIG. 2D)-ELF1 (E74 Like ETS Transcription Factor 1) and ZBTB7A (Zinc finger and BTB domain containing 7A) (FIG. 11A). Of these two, only one was found to be significantly dysregulated between MDD and controls: ZBTB7A, which showed significant upregulation in MDD OFC tissues at both the mRNA and protein level (FIGS. 2E-2F, FIG. 11B). ZBTB7A is a regulatory factor with pleiotropic effects (both repressive and activating) and has been shown to coordinate alterations in chromatin structure that are necessary for NF-κB dependent inflammatory gene expression in the context of several types of cancers (notably gliomas) and inflammatory conditions. However, its contribution to psychiatric disease has not yet been explored.

To confirm that ZBTB7A is differentially bound to chromatin in MDD OFC, footprinting analysis was carried out and ZBTB7A binding predictions were calculated within all identified OCRs. Consistent with motif enrichment analysis, 43.8% higher number of bound ZBTB7A sites that were specific to non-neuronal vs. neuronal cells were observed, with higher occupancy (3.4×) of ZBTB7A sites in non-neuronal MDD cases compared to controls, making it one of the top five most differentially bound TFs genome-wide between MDD and controls (FIGS. 2G, 11C). One illustrative example highlighting the importance of ZBTB7A is PRR5L (proline rich 5 like gene), a previously identified MDD biomarker gene involved in stress responsiveness, which displayed increased chromatin accessibility in MDD cases in multiple intronic OCRs. The most dysregulated OCR associated with this gene overlapped with two ZBTB7A binding sites (FIG. 2H), both of which displayed differential binding based on MDD vs. control status. Furthermore, gene targets of non-neuronal promoters (which were enriched for MDD-related genetic variants) and differentially expressed (DE) genes in MDD were enriched for ZBTB7A regulation and ZBTB7A-related processes (inflammation, NF-kB activation, and cholesterol synthesis) (FIGS. 21-2J, 6F-6G). Similarly, GO analysis performed on a published RNA-seq dataset from OFC of MDD patients vs. controls also revealed significant enrichment of ZBTB7A targets (FIG. 11D), as well as significant upregulation of ZBTB7A expression, indicates that altered ZBTB7A activity in OFC is observed across heterogeneous human MDD cohorts.

In order to examine which non-neuronal cell-types may be regulated by ZBTB7A activity in the context of MDD, GO analysis was performed. Overlapping MDD-specific OCRs with reference panels from human cell-type-specific ATAC-seq data revealed significant enrichment for astrocyte/microglia regulatory elements (these two cell-types were sorted together in the study) (FIG. 1E). GO analyses of ZBTB7A target genes (based upon ChIP-seq data) revealed robust enrichment for astrocyte-specific targets (FIG. 11E), second only to breast tissues. In addition, RNA-seq and FANS-ATAC-seq MDD profiles were significantly enriched for both ZBTB7A regulation and astrocyte cell-type specific expression (FIGS. 1C, 21-2J, 10A). In order to explore the possibility that ZBTB7A may be involved in regulation of gene targets associated with MDD-specific OCRs in human astrocytes, the effect of overexpressing ZBTB7A using a lentivirus with human primary cortical astrocytes was examined. In this astrocyte-enriched human cell culture system, ZBTB7A overexpression was found to significantly increase the expression of numerous genes regulated by MDD-specific OCRs in the human dataset, as compared to the RFP control, including prominent genes in the NF-kB pathway implicated by GSEA (FIGS. 11F-11H). Treating both cultured human and mouse astrocytes with lipopolysaccharide (LPS) to stimulate inflammation also resulted in a significant upregulation of ZBTB7A expression compared to saline, further linking this chromatin regulator to cellular activation pathways in astrocytes (FIGS. 111-11J).

In order to assess the potential for investigating ZBTB7A in stress responsivity, a preclinical animal model of chronic social defeat stress (CSDS) was used. The model and experiment involves 10 days of exposure sessions to a larger, aggressive mouse, and induces robust behavioral deficits, including reduced social interaction and reward insensitivity (FIG. 2K). Importantly, the CSDS paradigm also models natural variation in stress vulnerability, as approximately 30% of wild-type mice that go through the CSDS paradigm do not exhibit behavioral deficits related to chronic stress-termed stress-resilient (vs. stress-susceptible). Using this CSDS paradigm, Zbtb 7a protein was found to be upregulated in bulk OFC tissues from stress-susceptible subjects, but not control or stress-resilient animals, 48 hours after the last defeat session-indicating that Zbtb7a may be associated with behavioral susceptibility to chronic stress (n=8 control, n=11 stress-susceptible, n=9 stress-resilient) (FIG. 2L). Persistently increased OFC Zbtb7a expression was also observed in a separate cohort of stress-susceptible mice 21 days after CSDS (n=12 control, n=13 stress-susceptible), indicating that Zbtb7a upregulation is maintained after stress exposure (FIGS. 11K-11L). To determine whether chronic stress leads to Zbtb 7a upregulation specifically in astrocytes, Zbtb7a expression was examined in a previously generated astrocyte-specific Translating Ribosome Affinity Purification coupled to sequencing (TRAP-Seq) dataset (n=3 control, n=5 stress-susceptible, n=4 stress-resilient). Zbtb7a mRNA translation was found to be significantly upregulated in frontal cortical astrocytes of stress-susceptible mice, but not control or stress-resilient (FIG. 2M). Finally, Magnetically Activated Cell Sorting (MACs) was used to isolate astrocyte, neuron, and microglia-enriched cell fractions from the OFC of a separate CSDS cohort (n=4 control, n=4 stress-susceptible, with 3 pooled animals/n) (FIGS. 11M-110). Zbtb7a protein was found to be expressed at robust levels in mouse OFC astrocytes, and Zbtb7a mRNA expression was increased exclusively in astrocytes isolated from stress subjects vs. controls, with no significant differences observed in neurons or microglia (FIGS. 2N-2P, 11P-11R).

### Example 3: Knockdown of Zbtb7A in Rodent Ofc Astrocytes Rescued Alterations in Chromatin Accessibility Associated with Chronic Stress

In order to determine whether astrocyte-specific manipulation of Zbtb 7a would alter chromatin accessibility patterns in the context of chronic stress, Zbtb 7a inhibition experiments were performed. To knock down (KD) Zbtb7a specifically in astrocytes in the rodent brain, a Zbtb7a-targeting miRNA-GFP construct was packaged into an rAAV6 vector under the control of the astrocyte-specific GFAP promoter (FIGS. 3A-3B). Preferential expression of the GFP transgene expression in GFAP+ cells was confirmed using magnetic-activated cell sorted (MACs)-astrocytes (FIGS. 12A-12G), the efficiency of Zbtb7a KD was validated in transduced OFC tissue (FIG. 3B).

OFC of male mice were stereotactically transduced with the AAV6-GFAP-Zbt-miR (Zbt-KD) vector, or a miRNA-negative-GFP (GFP) virus as a control, prior to the CSDS paradigm, with half of each viral group being assigned to either control or CSDS conditions (FIG. 3A). ATAC-seq was performed on MACs-isolated astrocytes from virally-infected OFC tissues (n=4 GFP control, n=4 Zbtb7a KD control, n=5 GFP chronic stress, n=5 Zbtb7a KD chronic stress, with each n composed of 3 pooled OFC samples). Differential analyses revealed that Zbtb7a KD rescued astrocyte-specific chromatin accessibility patterns induced by chronic stress, with 42.3% (603/1391) of up events and 65.5% (2044/3117) of down events displaying opposite accessibility compared to GFP chronic stress (FIGS. 3C-3E, 12H-12J). Rescued OCRs were enriched for Zbtb7a regulation and chromatin binding, as well as pathways involved in astrocyte reactivity, including calcium signaling, ion homeostasis, ECM alterations, and cellular morphogenesis (FIG. 3F). Furthermore, although the majority of differential peaks were located in intergenic enhancers/repressors, promoters with increased accessibility in chronic stress were enriched for Zbtb7a targets, as were less accessible promoter regions in Zbtb7a KD chronic stress vs. GFP chronic stress, indicating that these chromatin profiles reflect Zbtb7a regulatory activity in astrocytes (FIG. 12K).

### Example 4: Knockdown of Zbtb7A in Rodent Ofc Astrocytes Reversed Cell-Type Specific Inflammatory Gene Expression and Behavioral Deficits Associated with Chronic Stress

To explore if Zbtb7a KD in the context of chronic stress affects astrocyte-specific gene expression, RNA-seq profiling was used for MACs-isolated astrocytes from Zbtb7a KD vs. GFP groups (+/−) CSDS (n=4 GFP control, n=4 Zbtb7a KD control, n=4 GFP chronic stress, n=5, Zbtb7a KD chronic stress, with 3 pooled OFC astrocyte fractions per n). Transcriptome-wide relationships in the RNA-seq dataset were compared to evaluate the effect of Zbtb 7a KD in the context of chronic stress. Threshold-free Rank-Rank Hypergeometric Overlap (RRHO) analysis revealed a robust pattern of anti-correlated gene expression between Zbtb7a KD stress vs. GFP-stress and GFP-stress vs. GFP controls, showing that Zbtb7a KD reverses astrocyte gene signatures of chronic stress and maintains a more similar profile to controls (FIGS. 3G-3H, FIG. 12L). Unsupervised clustering of chronic stress DE genes (2513 genes, FDR<. 1) showed that both Zbtb7a KD stress and Zbtb7a KD controls display an intermediate gene expression phenotype that clusters between controls and chronic stress (FIG. 31). In addition, Zbtb 7a KD chronic stress RNA-seq profiles correlated positively with Zbtb7a KD chronic stress chromatin accessibility changes detected in our astrocyte-specific ATAC-seq, and exhibited a reversed pattern of anti-correlation with chronic stress OCRs (FIGS. 12M-12N).

Transcriptome-wide GSEA demonstrated that Zbtb7a KD reversed astrocytic upregulation of inflammatory response gene sets (including NF-kB) induced by chronic stress (FIG. 3J). Differential expression analysis showed that chronic stress upregulated pathways associated with astrocyte reactivity (e.g. cell motility and morphological remodeling), while downregulating genes involved in critical astrocyte functions such as metabolic homeostasis and regulation of synaptic signaling-pathways that were rescued by Zbtb7a KD (FIGS. 3K-3L). Approximately 96% (112/117) of DE genes (FDR<0.1) between the Zbtb7a KD stress and GFP stress groups were rescued (FIG. 120). Among the genes reversed by Zbtb7a KD were the water channel Aqp4 and the glutamate clearance transporter Eaat2, both of which modulate neuronal excitability through maintenance of perisynaptic potential, and have been found to be dysregulated in chronic stress. To determine if Zbtb7a KD affects gene expression signatures more globally, RNA-seq was performed on bulk OFC tissues from a separate cohort of animals (n=4 GFP control, n=4 Zbtb7a KD control, n=8 GFP chronic stress, n=8 Zbtb7a KD chronic stress, 1° F.C sample/n). Bulk RNA-seq profiles recapitulated the findings of Zbtb 7a KD reversing chronic stress-related transcriptomic patterns, including inflammatory gene sets (FIGS. 12P-12S). The most significantly changed DE genes between the two stress conditions in bulk tissues were enriched for genes involved in synaptic activity and glutamatergic transmission (FIG. 12U), suggesting that neuronal function may also be affected by astrocytic Zbtb7a KD in chronic stress.

In order to examine non-cell autonomous effects of astrocytic Zbtb7a KD, MACs-isolated OFC neurons from the same cohort of Zbb7a KD vs. GFP animals (+/−CSDS) (n=4 GFP control, n=4 Zbt-KD control, n=4 GFP chronic stress, n=5, Zbt-KD chronic stress, with 3 pooled OFC neuronal fractions per n) were profiled. Comparing these data with the astrocyte-specific profiles, both astrocyte and neuronal fractions demonstrated cell-type specific expression of respective population markers (FIGS. 13A-13B). While both RRHO and clustering of DE genes showed less significant reversals between the two stress groups in neurons than was observed in astrocytes, a similar pattern of globally rescued gene expression and inflammatory gene sets in the Zbtb7a KD chronic stress vs. chronic stress conditions was observed (FIGS. 13C-13G). In addition, Zbtb7a KD specifically reversed chronic stress-induced upregulation of glutamate transmission and genes associated with neuronal excitability in neurons (FIGS. 13H-131), indicating that inflammatory astrocyte activation during chronic stress leads to a loss of normal astrocytic homeostatic processes that may have downstream consequences on OFC neuronal activity.

In a separate cohort of CSDS male mice, astrocyte-specific Zbtb 7a KD in OFC attenuated chronic stress-induced deficits in social interaction, with no significant changes observed in Zbt-KD control mice (FIG. 3M). Zbtb7a KD also rescued anhedonia-like behavior post-CSDS in two different measures of saccharin reward sensitivity: a Pavlovian cue-reward association task, in which mice learn to associate a signal light with reward delivery, as well as an operant reward learning task requiring lever-pressing in response to a cue light to receive rewards (FIGS. 3N-3Q). Together, these findings indicate dysregulated astrocyte pathways in human MDD, and highlight the possibility of targeting Zbtb 7a therapeutically to address OFC-mediated deficits associated with chronic stress exposure.

### Example 5: Overexpression of Zbtb7A in Rodent Ofc Astrocytes Increased Vulnerability to Stress-Related Behavioral Deficits

In order to explore the mechanistic role of astrocytic Zbtb7a in stress vulnerability and to test if increased Zbtb7a is sufficient to elicit a behavioral stress response, an overexpression construct for ZBTB7A was packaged into the same AAV6-GFAP viral vector used for KD experiments (FIGS. 4A-4C, 14A-14B). A mild, single-day acute stressor paradigm, termed sub-threshold social defeat (SSDS), which is not sufficient to induce behavioral deficits in wild-type animals, was used in order to assess whether ZBTB7A overexpression in OFC astrocytes potentiates a pro-susceptibility effect (FIG. 4D).

In these SSDS mice, astrocyte-specific ZBTB7A overexpression increased vulnerability to behavioral deficits following the acute stressor compared to GFP mice, including heightened social avoidance, anhedonic sucrose consumption and reward sensitivity, and despair-like behaviors in the forced swim test (with no significant differences in anxiety-like behaviors) (FIGS. 4E-4G, 14C-14E). Zbtb7a overexpression+acute stress also induced specific deficits in reward-based reversal learning (FIGS. 4H-4I). In contrast, GFP-expressing mice displayed distinct adaptive behaviors in response to acute stress, with no significant differences between GFP SSDS mice and GFP control observed. Furthermore, ZBTB7A overexpression itself did not affect stress-related behaviors in ZBT-overexpression control mice. These findings demonstrate that ZBTB7A overexpression in OFC astrocytes elicits increased vulnerability to stress-related behavioral dysfunction after a mild stressor, inducing a behavioral phenotype more similar to that of mice put through a chronic stress paradigm.

### Example 6: Zbtb7A Overexpression in Astrocytes Induced Transcriptome-Wide Alterations in Gene Expression and Chromatin Accessibility

In order to examine the broader impact of astrocyte-specific ZBTB7A overexpression vs. GFP on gene expression in acute stress SSDS animals, bulk RNA-seq was performed on virally transduced OFC tissues (n=5 GFP control, n=5 ZBT-overexpression control, n=7 GFP SSDS, n=6 ZBT-OE SSDS, 1° F.C per n). RRHO and unsupervised clustering of DE genes (1903 genes, FDR<0.1) revealed a reversed pattern of transcription between the two SSDS groups, while the ZBT control condition exhibited a positive correlation with GFP SSDS-indicating that ZBT overexpression in the absence of a mild stressor does not disrupt the overall transcriptomic state, in agreement with behavioral data (FIGS. 4J-4L). ZBTB7A overexpression+acute stress significantly increased NF-κB inflammatory pathways, astrocyte reactivity (e.g., defense response, ECM alterations), and cytokine release (processes that were also significantly enriched in human MDD-specific ATAC-seq OCRs and RNA-seq) (FIG. 4M). Flow cytometry analysis confirmed that while the overall number of astrocytes or microglia was not changed between conditions, astrocytic ZBTB7A overexpression+acute stress significantly increased functional measures of neuroinflammation in the OFC, such as increased percentages of microglia expressing activated markers (FIGS. 14F-14G). Inflammatory gene sets were downregulated in the GFP SSDS vs. GFP control mice, demonstrating that behavioral resilience after an exposure to acute stress involves pro-adaptive transcriptional responses that are reversed by ZBTB7A overexpression in OFC astrocytes. GO analysis revealed that ZBTB7A overexpression+acute stress induces similar changes to both human MDD RNA-seq and mouse chronic stress, including enrichment for ZBTB7A targets, as well as modulation of trans-synaptic signaling, calcium signaling, and neuronal differentiation (FIG. 14H).

Profiling of MACs-isolated OFC astrocytes from a separate cohort of mice (n=4 GFP SSDS, n=4 ZBT-OE SSDS) revealed robust changes in accessibility (6,094 differentially accessible regions), which also correlated significantly with observed differential gene expression (715 DE genes, FDR<0.1) (FIGS. 14I-14M). ZBTB7A overexpression-induced OCRs overlapped significantly with chromatin regions that were less accessible in chronic stress (and subsequently more accessible in Zbt-KD+chronic stress)—suggesting that ZBTB7A-mediated repression may be a central regulatory feature in astrocyte dysfunction during chronic stress (FIGS. 4N-4P). ZBTB7A overexpression+acute stress induced similar alterations in gene expression as those observed in chronic stress; chromatin and gene expression changes were associated with astrocyte reactivity pathways, including cellular morphology, ion homeostasis, and immune activation (FIGS. 4N, 14L). Neuronal RNA-seq profiles showed an increase in inflammatory response, as well as decreases in ion transport regulation and GABAergic synaptic transmission, indicating that astrocytic ZBTB7A overexpression may induce behavioral stress susceptibility through disinhibition of OFC neuronal activity (FIGS. 14N-140).

### Example 7: Zbtb7A Overexpression in Ofc Astrocytes Potentiated Synaptic Transmission

In order to investigate whether astrocytic ZBTB7A overexpression affected functional measures of synaptic transmission in mouse OFC following SSDS compared to GFP, ZBTB7A was overexpressed in astrocytes. A GFAP-driven AAV6 virus was used to overexpress either ZBTB7A or GFP in OFC astrocytes (+/−) SSDS, followed by electrophysiological slice recordings to assess the effects of ZBTB7A overexpression on synaptic transmission post-exposure to a SSDS mild stressor (FIG. 5A). An input-output (I-O) curve of field excitatory postsynaptic potentials (fEPSPs) in response to presynaptic stimuli was plotted. A significant increase in I-O curves was observed in ZBT-OE SSDS vs. GFP SSDS, suggesting that ZBTB7A overexpression in astrocytes induces potentiation of postsynaptic responses following an acute stress (FIGS. 5B-5C). Stimulation protocols were applied to assess the dynamics of presynaptic vesicle mobilization and release. During the stimulation train, a significant difference in the fEPSP amplitude between ZBT SSDS vs. GFP SSDS was detected, indicating a faster depletion of the readily releasable pool of vesicles, which is correlated with higher probability of presynaptic release (FIGS. 5D-5E). Together, these data indicate that astrocytic ZBTB7A overexpression+acute stress induces increased OFC neuronal excitability.

In order to determine if the observed neuronal changes are associated with alterations in astrocyte signaling, calcium imaging was performed in 2D mouse primary mixed cortical cultures of neurons and glia (including astrocytes), using the genetically encoded calcium indicator GCaMP6f. Primary mixed cultures were transduced with AAVI-hSyn-GCaMP6f or AAV5-gfaABCID-cyto-GCaMP6fto to ensure selective expression solely in neurons or in astrocytes, respectively (FIGS. 15A-15E). To elicit a subthreshold-like adaptive cellular response, cultures were treated with a low-dose of LPS (LPSlow), approximately 1-10% of a typical inflammatory dose. In GCaMP6f control astrocytes, ZBT-overexpression significantly increased calcium signaling after LPSlow treatment compared to the empty vector control virus, suggesting that ZBTB7A overexpression disrupts adaptive astrocyte plasticity to subthreshold stressful stimuli (FIG. 15B). Furthermore, neuronal calcium events, which are a proxy marker for action potentials, were also significantly increased in co-cultures treated with the astrocyte-specific ZBT-overpexression virus+LPSlow compared to empty vector controls (FIG. 15C). These data demonstrate that ZBTB7A overepxression in astrocytes leads to increased astrocyte activity, and impairs both astrocyte and neuronal adaptations to mild stressor stimuli.

### Example 8: Ofc Neuronal Hyperexcitability Associated with Astrocyte Dysfunction Mediates Behavioral Vulnerability to Stress

In order to determine if increased OFC neurotransmission is provides a functional link between ZBTB7a-mediated astrocyte reactivity and behavioral vulnerability to stress, an inhibitory Designer Receptors Exclusively Activated by Designer Drugs (DREADD)-based chemogenetic approach was used to inhibit OFC neurons while simultaneously overexpressing ZBTB7A in surrounding astrocytes.

It was confirmed that I.P. injection of the DREADD agonist Deschloroclozapine52 (DCZ) on its own did not alter previously observed patterns of behavioral deficits in ZBT overexpression vs. GFP mice post-stress in absence of the DREADD, and that the Gi DREADD without DCZ agonist activation does not affect previously observed SI phenotypes (FIGS. 15F-15H). The SSDS paradigm was performed on a separate cohort of male mice that were injected intra-OFC with the pAAV-hSyn-hM4D (Gi)-mCherry vector to express the inhibitory Gi DREADD, in combination with either the AAV-GFAP-ZBTB7A overexpression construct or the rAAV6-GFAP-GFP empty control vector (FIG. 5F). Both groups underwent the SSDS paradigm. Prior to the social interaction test, half of each viral group was injected with either vehicle or the DREADD agonist DCZ to activate the inhibitory Gi DREADD and silence OFC neuronal firing (FIG. 5G). In the Gi+vehicle-injected mice, ZBT overexpression+acute stress resulted in reduced social interaction behavior vs. GFP SSDS, as previously observed. However, for DCZ-injected mice, in which Gi DREADDs were activated prior to the SI test by DCZ, a significant rescue in SI deficits was observed, indicating that silencing of OFC activity prior to the social interaction test led to amelioration of the pro-stress susceptibility effects of astrocyte-specific ZBT overexpression (FIG. 5H). Together, these findings point to an astrocytic ZBTB7A-induced increase in synaptic connectivity driving maladaptive stress susceptibility, which is consistent with clinical reports of astrocyte dysfunction and neural hyperactivity in human MDD OFC.

### Example 9: Glutamate Clearance Following Chronic Stress

To investigate synaptic glutamate clearance by astrocytes in the context of chronic stress, the decay rate (Tau) of NMDA receptor-dependent excitatory post-synaptic potentials (mEPSCs) were measured from individual neurons in mouse acute brain slices. The mice used in this experiment expressed an empty vector control (GFP) in astrocytes specifically, and measurements were performed in glutamatergic pyramidal neurons from mice exposed to stress conditions and control conditions. Whole-cell patch-clamp recordings were performed from excitatory neurons in orbitofrontal cortex brain slices. The decay kinetics of the NMDA-dependent EPSPs provide an estimate of how rapidly synaptically released glutamate is taken up by astrocytes. The time constant tau (t) was calculated for stressed conditions and control conditions. FIG. 16 is a plot of Tau (ms) for stressed conditions and control conditions, showing a significantly increased Tau (ms) for the stressed condition. These data indicate that chronic stress leads to a reduction in glutamate clearance (i.e., increased Tau-decay rate) from the synapse in orbitofrontal cortex.

### Example 10: Zbtb7A Knockdown in Chronic Stress

To investigate the effect of ZBTB7A knockdown on glutamate clearance by astrocytes in the context of chronic stress, the decay rate (Tau) of NMDA receptor-dependent excitatory post-synaptic potentials (mEPSCs) are measured from individual excitatory neurons in mouse acute brain slices expressing either an AAV vector comprising an miRNA targeting Zbtb7a or an empty vector control (GFP) in astrocytes. Whole-cell patch-clamp recordings are performed from neurons in orbitofrontal cortex brain slices. The decay kinetics of the NMDA-dependent EPSPs provide an estimate of how rapidly synaptically released glutamate is taken up by astrocytes. The time constant tau (t) is calculated for stressed conditions and control conditions. This experiment indicates that Zbtb7a knockdown reverses the decrease in glutamate clearance that is observed in the context of chronic stress.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a psychiatric disorder, the method comprising:
identifying a subject as having the psychiatric disorder; and
administering to the subject an effective dose of a composition that reduces levels of ZBTB7A in cells of the subject.

2. The method of claim 1, wherein the psychiatric disorder is one of Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition.

3. The method of any one of claims 1-2, wherein the composition comprises an inhibitory agent that targets ZBTB7A in cells of the subject.

4. The method of any one of claims 1-3, wherein the composition comprises an inhibitory agent that reduces expression of ZBTB7A in cells of the subject.

5. The method of any one of claims 3-4, wherein the inhibitory agent is an antisense oligonucleotide (ASO).

6. The method of any one of claims 3-4, wherein the inhibitory agent is a nucleic acid-guide nuclease.

7. The method of any one of claims 3-4, wherein the inhibitory agent is an RNAi agent.

8. The method of claim 7, wherein the RNAi agent is an siRNA, an shRNA, or a miRNA.

9. The method of claim 7, wherein the RNAi agent is a miRNA encoded by a DNA sequence comprising a sequence selected from the group consisting of SEQ ID NO: 3; SEQ ID NO: 4; SEQ ID NO: 5; SEQ ID NO: 6; SEQ ID NO: 7; SEQ ID NO: 8; SEQ ID NO: 9; or SEQ ID NO: 10.

10. The method of claim 9, wherein the miRNA is expressed by a recombinant adeno-associated (rAAV) virus.

11. The method of any one of claims 3-10, wherein the inhibitory agent is administered with a pharmaceutically acceptable carrier and/or diluent.

12. The method of any one of claims 1-11, wherein the cells of the subject are in the orbitofrontal cortex (OFC) of the subject.

13. The method of claim 12, wherein the cells of the subject are astrocyte cells.

14. The method of any one of claims 1-13, wherein levels of ZBTB7A are reduced by 10%, 20%, 30%, 40%, 50%, 60%, 70%, 80%, 90%, or 100% in the cells of the subject.

15. A method of treating a psychiatric disorder, the method comprising administering to a subject having the psychiatric disorder an effective dose of a composition comprising a recombinant adeno-associated virus (rAAV) comprising a ZBTB7A-targeting inhibitory RNA-encoding DNA.

16. The method of claim 15, wherein the psychiatric disorder is one of Major Depressive Disorder (MDD), Bipolar Disorder, Dysthymia, Post-traumatic Stress Disorder (PTSD), Substance-induced mood disorder, Generalized Anxiety Disorder, Social Phobia, Panic Disorder, or Mood Disorder Due to a General Medical Condition.

17. The method of any one of claims 15-16, wherein the ZBTB7A-targeting inhibitory RNA-encoding DNA comprises a sequence selected from the group consisting of SEQ ID NO: 3; SEQ ID NO: 4; SEQ ID NO: 5; SEQ ID NO: 6; SEQ ID NO: 7; SEQ ID NO: 8; SEQ ID NO: 9; or SEQ ID NO: 10.

18. The method of any one of claims 15-17, wherein the recombinant AAV is administered by parenteral, intravenous, intrathecal, introcerebroventricular, or cisterna magna administration.

19. The method of claim 18, wherein the intrathecal administration is by lumbar puncture.

20. A recombinant adeno-associated virus comprising a ZBTB7A-targeting inhibitory RNA-encoding DNA comprising a sequence selected from the group consisting of SEQ ID NO: 3; SEQ ID NO: 4; SEQ ID NO: 5; SEQ ID NO: 6; SEQ ID NO: 7; SEQ ID NO: 8; SEQ ID NO: 9; or SEQ ID NO: 10.

21. A composition comprising the recombinant adeno-associated virus of claim 20 and a pharmaceutically acceptable carrier.

22. A method of inhibiting expression of ZBTB7A in a cell comprising contacting the cell with the recombinant AAV of claim 20, wherein the expression of the ZBTB7A-targeting inhibitory RNA inhibits expression of ZBTB7A in the cell.

23. The method of claim 22, wherein the cell is in the orbitofrontal cortex (OFC) of a subject.

24. The method of claim 23, wherein the cell is an astrocyte cell.


SEQUENCE LISTING (11 sequences)

SEQ ID NO: 1 - RNA, 6437 nt, Homo sapiens
  source 1..6437: mol_type = mRNA
ggagaaaccaggaagggagtcgcgagcatcatacggggctttggctgtactgtacagttactgtaggggcagtgacgccgccgccgccgcgcgagggagcgaccagggcagcgcgacgcgggcagagcgagcgccgcggaccccggaggcgagcggccgagggagcccagtcccaacttcgggccccggccccgcgcgccccggcccggccccgcgaggtctcggcgcggaagatggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcagatcctggcggccgacgcgggcgccgacgccgggcagctggaccttgtagatcaaattgatcagcgcaacctcctccgcgccaaggagtacctcgagttcttccagagcaaccccatgaacagcctgccccccgcggccgccgccgccgctgccagcttcccgtggtccgcctttggggcgtccgatgatgacctggatgccaccaaggaggccgtggccgccgctgtggccgccgtggccgcgggcgactgcaacggcttagacttctatgggccgggccccccggccgagcggcccccgacgggggacggggacgagggcgacagcaacccgggtctgtggccagagcgggatgaggacgcccccaccgggggtctctttccgccgccggtggccccgccggccgccacgcagaacggccactacggccgcggcggagaggaggaggccgcctcgctgtcggaggcggcccccgagccgggcgactctccgggcttcctgtcgggagcggccgagggcgaggacggggacgggcccgacgtggacgggctggcggccagcacgctgctgcagcagatgatgtcatcggtgggccgggcgggggccgcggcgggggacagcgacgaggagtcgcgggccgacgacaagggcgtcatggactactacctgaagtacttcagcggcgcccacgacggcgacgtctacccggcctggtcgcagaaggtggagaagaagatccgagccaaggccttccagaagtgccccatctgcgagaaggtcatccagggcgccggcaagctgccgcgacacatccgcacccacacgggcgagaagccctacgagtgcaacatctgcaaggtccgcttcaccaggcaggacaagctgaaggtgcacatgcggaagcacacgggcgagaagccgtacctgtgccagcagtgcggcgccgcctttgcccacaactacgacctgaagaaccacatgcgcgtgcacacgggcctgcgcccctaccagtgcgacagctgctgcaagaccttcgtccgctccgaccacctgcacagacacctcaagaaagacggctgcaacggcgtcccctcgcgccgcggccgcaagccccgcgtccggggcggggcgcccgaccccagcccgggggccaccgcgacccccggcgcccccgcccagcccagctcccccgacgcccggcgcaacggccaggagaagcactttaaggacgaggacgaggacgaggacgtggccagccccgacggcttgggccggttgaatgtagcgggcgccggtggaggaggtgacagcggaggtggccccggggccgccaccgacggtaacttcacagccggactcgcctaaaaaccaaaaagagaaaacagaaacccgagaaagagagagacagagagagagaaaaaaaatcacccaccacccccccaaaaacacaaaaaaagaaaatctatctatatacagatatctatatctatatatatatatacagatatatatatatgacgcgtcacagaatctagggtagcgctttctcagatttccctcctttctgacgtttttctccctccgcaggggccccggccctccctggctccccttccccccaccaccccatcgctgggtttcggggcttggtttggggttttttgtaggacacaaggaatccgagaccccgcacagccccctgggcacccggcatggggcctggggcccgatccgaggccctgggctggggggagggtagacgtgggggcgctggggggggactggggtgggcttttaatttcctcccctcgctggtttctatgagtctttcagacaagaccttaaatgatttctgtctgctctgagcggacgttaaaatgggcccccgtcccccgacccgcaccctccttcctcagggcacttactaagggaggggtctccctctccatctccccagtggcctccccgcctccaaccctgcctgcggcctccccccgtcgcccaccccacgtctcctggccactgagacacaaacctatttatttctaggcctggagaaaggagatcggactggggttcccggtggggcgccaggatggctcctgggggtgctcctgccgccttccttcacggcacttacaaccggcgggacccccagggaccacccctcagggcgcccccccacccccgcccggtccacctagacccccacgtttggagattcaaaacttctgtcttcgtcctctcccccgagccccctctcccaaatttttaaagcactttttagatttttttttctctttcctccttaaaaacaaaatttatatatagatatatatatatatataaataatatacttttcctcagaggagcaggcaacagtgtgggataaacagagtcacgatcagaggaaccccagggtctggtgatggcagggatggggggagagagagaaaatccacaaattccaatgtcacaaaagcaataaaacaaactagaaaaaaaaaaggttttacaaaatgaaaggaaggaaaaaaaaaaaggcaaccaaccacattagaagtcttggcactttgtaacggaacgggtactacactttatcttaattcttaatttaaaaacatgtttacaagttacaaccaacttctatgaaaagttgaaaagacaaaaaaaaaaaaaaaaagcgagcgagagagagagcgagagagagagcgagagcagaagaaattcctaaaagtcgatttatttttgtacaaaataataaaaaaaaaaacccaccacaaacgtagaatccacttctgttccccaaaaagcgagaaggggggttcaggaggaagccatcgcaggggacctgggagacgccccgaggtgtttgtgcttcacccccagacgtcagcctcgaaggcaggactgtggggtgttcgtgctgtgttccccccgctccccctttctgtccccttttttggttctgacgtgaagaggtcttagcgcccgcttctgtccacggggtctctccttcctcctccctagctcagggatgggccttccagccggagcaccccgatccccatccggcaccccccaatcccccaacacgcctgtccctcccgcatggccaccaaggagctggaccttggatgcgcctaccctgctgaggtgggtgacaggggccccccacctccagggccttagaaccaccgcccctctccccaccccaggcacccctctttttactcaaaggcactgactgtaatccagggggactgggacctgcctccccccaacctctggctcccacaaggcccggtgttgaccgagccacaggccacggacaggggccggggttggggagactatgtcgccagatgccaggacgccctcaccccgtttgcatatgcaatgctagcatgggaccccgaaaatagacgctctgctgcactgagacttcttgtcaatgcccaaccggcgggggggtgtctccctgcccccgacccccccatacccccttctctgtgacacacacatcttctcgtctctttttctttcattgttaaagggaagctttttaagaaggcaattttcatattgtttctacaggatggttttggttcccttcccttcccacccccccttaagcctgtcagccccctccaaatgtctcaggatcccccctctcccctggggctgggtgacagcaccccggctgcgttcacaccccagtgtcacagggcgagctgttctggagagaaaaccatctgtcgtggctgagcggggagcttgaacacccaggccagggacacccctccccagctcccagagaggccccctgaggggtgagccctctttccaccttcccctatccatgcaccccctcgcaataaaaccaactctaaaatcacagctgtcgtcctagccagtgggggcgaccggacttggggggtggagccctctgggacttccgtaggaacaagggctgcggcccaccgcgacacttacacagacctcggggattgcactaaaccctcgttcctagctccgcactcagcttcgcctgtcctgcccgcccactttgccttaactacccgcccgtcctgggggccacagcctctgcatgggcccagagccgggacccccccagcccagccccgccctccccagactccgcgcaatcacatactgtatatagacgtgaatcgattttatttttattctttaaattaaggtcgtgataaagtgttgccaaagatacctgctgaattctcgcgtttcaggaaacaaacaaacaaaaaaaaatgatatttgaggagggtcgtgttgactccatatgaaaggacacagctcaaagcttttttgtttggttgtttggggttttttgtgttttctttttttggggtgttttttttttaactgcctggtacaaaaaaaaaaagagaaaaaaaaaaaagaaaaacaatgcgaaattgttatttccattctcatggtgaagttgcgtggacgcgtgtgtgcgtgtgtgcaagagagcgggagtgaggtccaggctggggttggggggcttcaggcgggggcgcccgggggccggggaggtggccgggccggagcccccgtctgcagtgccccccagcctgccgggcccaggagagagagagaagcatctttgctactagctgttgctgctacctgcctctgccccccgacgccccccgccttttgagattaaggaaaaaaaaaaaaagtcaaaaaagtttttaaaaatgaaaaaaaaaaattataaaccagtgaatgtaaaatgccggagcaggcccggcctggcatgggtgtggacctgcagccaggcaggctcgagcgggcgataccaaagtctgcccccccaccattgtggccatgcagtcctgtcactgtctttttgcttccttccgaggggggtcccccagcctcttccagggtcttcccctggaagtgggcggctgcagggaaggtgggggacaggggtctttgcacgattcagaccccggggccgtggcaggagcggtcacctcacaggtggtgacactgaggcaggggcctcggggtgccccctcccgcccggcaaccagaatggttggaggcaagacagagagaatgaaaggaaaaacagaagaaaaaaaaatattaaaaaccaacaaaaaaagcaaaaatcctattttttgagaaagaaagatatttatatttgcagttttattttaaaaagttatttaagttgaagcagccttcctggaggtgggggggggggggtggtgggtggctggcgcaggacgggtcaggggcctggaggctgggggtgccccaggagctacaacctcagagttaagactagctcgcattaaatacatagatttacggggggggggggggggggccgggcccagggggtggagggggccagggagacccccatccctcgccggggctgcctggaggctgtggaccaggatccgatgcccaggtcccgccccccaccccaccccaggcccagaatcgaggtgccttggactttggaggggccaggcctggtgaatggggggcggggcggcgccctcagggtacagagcacagacagatagacattccagagactgtattgagagtctttataaagtgtgggagatttaaaaaaaaaaaaaactgataaaaatgcactttttgggagtggggagggagaagctttaaaagtaataaaaaacaaacaaaaacacaaaagatgaaaaaacaaaaaaattcatttttcttgtacataaaaaaaaaaaaagaaccactaaacgcagcctgttacgacc

SEQ ID NO: 2 - protein, 584 aa, Homo sapiens
MAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQILAADAGADAGQLDLVDQIDQRNLLRAKEYLEFFQSNPMNSLPPAAAAAAASFPWSAFGASDDDLDATKEAVAAAVAAVAAGDCNGLDFYGPGPPAERPPTGDGDEGDSNPGLWPERDEDAPTGGLFPPPVAPPAATQNGHYGRGGEEEAASLSEAAPEPGDSPGFLSGAAEGEDGDGPDVDGLAASTLLQQMMSSVGRAGAAAGDSDEESRADDKGVMDYYLKYFSGAHDGDVYPAWSQKVEKKIRAKAFQKCPICEKVIQGAGKLPRHIRTHTGEKPYECNICKVRFTRQDKLKVHMRKHTGEKPYLCQQCGAAFAHNYDLKNHMRVHTGLRPYQCDSCCKTFVRSDHLHRHLKKDGCNGVPSRRGRKPRVRGGAPDPSPGATATPGAPAQPSSPDARRNGQEKHFKDEDEDEDVASPDGLGRLNVAGAGGGGDSGGGPGAATDGNFTAGLA

SEQ ID NO: 3 - DNA, 64 bp, synthetic construct
  source 1..64: mol_type = other DNA
tgctgatcatcagccgccagaatctggttttggccactgactgaccagattctcggctgatgat

SEQ ID NO: 4 - DNA, 64 bp, synthetic construct
  source 1..64: mol_type = other DNA
cctgatcatcagccgagaatctggtcagtcagtggccaaaaccagattctggcggctgatgatc

SEQ ID NO: 5 - DNA, 64 bp, synthetic construct
  source 1..64: mol_type = other DNA
tgctgtagaagtccaagccattgcaggttttggccactgactgacctgcaatgttggacttcta

SEQ ID NO: 6 - DNA, 64 bp, synthetic construct
  source 1..64: mol_type = other DNA
cctgtagaagtccaacattgcaggtcagtcagtggccaaaacctgcaatggcttggacttctac

SEQ ID NO: 7 - DNA, 64 bp, synthetic construct
  source 1..64: mol_type = other DNA
tgctgatagaagtccaagccattgcagttttggccactgactgactgcaatggtggacttctat

SEQ ID NO: 8 - DNA, 64 bp, synthetic construct
  source 1..64: mol_type = other DNA
cctgatagaagtccaccattgcagtcagtcagtggccaaaactgcaatggcttggacttctatc

SEQ ID NO: 9 - DNA, 64 bp, synthetic construct
  source 1..64: mol_type = other DNA
tgctgtgcaccttcagcttgtcctgtgttttggccactgactgacacaggacactgaaggtgca

SEQ ID NO: 10 - DNA, 64 bp, synthetic construct
  source 1..64: mol_type = other DNA
cctgtgcaccttcagtgtcctgtgtcagtcagtggccaaaacacaggacaagctgaaggtgcac

SEQ ID NO: 11 - DNA, 60 bp, synthetic construct
  source 1..60: mol_type = other DNA
gaaatgtactgcgcgtggagacgttttggccactgactgacgtctccacgcagtacattt